(19) United States
(12) Patent Application Publication
(10) Pub. No.: US 2026/0365663 A1
(43) Pub. Date: Nov. 19, 2026

(54) EMOPAMIL-BINDING PROTEIN INHIBITORS AND USES THEREOF

(71) Applicant: BIOGEN MA INC., Cambridge, MA (US)

(72) Inventors: Felix Gonzalez Lopez de Turiso, San Diego, CA (US); Martin Himmelbauer, Cambridge, MA (US); John H. Jones, Framingham, MA (US); Vatee Pattaropong, Bedford, MA (US); Robin J. PRINCE, Sharon, MA (US); Zhili Xin, Cambridge, MA (US)

(21) Appl. No.: 18/997,557

(22) PCT Filed: Jul. 20, 2023
(86) PCT No.: PCT/US2023/028271

§ 371 Date: Jan. 22, 2025

Related U.S. Application Data
(60) Provisional application No. 63/391,578, filed on Jul. 22, 2022.

(51) Int. Cl. C07D 491/107; A61K 31/397; A61K 31/4155; A61K 31/443; A61K 31/4433; A61K 31/4525; A61K 31/454; A61K 31/4545; A61K 31/506; A61K 31/5377; C07D 205/12; C07D 401/04; C07D 401/12; C07D 403/12; C07D 405/14; C07D 519/00

(52) U.S. Cl. CPC C07D 491/107; A61K 31/397; A61K 31/4155; A61K 31/443; A61K 31/4433; A61K 31/4525; A61K 31/454; A61K 31/4545; A61K 31/506; A61K 31/5377; C07D 205/12; C07D 401/04; C07D 401/12; C07D 403/12; C07D 405/14; C07D 519/00

(57) ABSTRACT

Provided are compounds of the Formula (I): or pharmaceutically acceptable salts thereof, which are useful for the inhibition of EBP and in the treatment of a variety of EBP mediated conditions or diseases, such as multiple sclerosis.

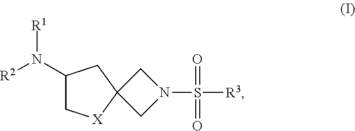

### RELATED APPLICATIONS

This application claims priority to U.S. Provisional Application No. 63/391,578 filed on Jul. 22, 2022. The entire contents of the foregoing application are expressly incorporated herein by reference.

### FIELD OF THE INVENTION

The present disclosure relates to inhibitors of Emopamil-Binding Protein (EBP), and pharmaceutically acceptable salts thereof, compositions of these compounds, processes for their preparation, their use in the treatment of diseases, their use in optional combination with a pharmaceutically acceptable carrier for the manufacture of pharmaceutical preparations, the use of the pharmaceutical preparations in the treatment of diseases, and methods of treating diseases comprising administering the EBP inhibitor to a warm-blooded animal, especially a human.

### BACKGROUND OF THE INVENTION

Emopamil-Binding Protein (EBP) is a Δ8-Δ7 sterol isomerase enzyme which isomerizes the double bond in sterol molecules, moving the double bond from the 8-9 position to the 7-8 position. Specifically, EBP converts either zymostenol to lathosterol, or zymosterol to dehydrolathosterol, during the biosynthesis of cholesterol (Silve et al., 1996, J Biol Chem. 271 (37), 22434-22440). It has been shown that an accumulation of 8-9 unsaturated sterols activates oligodendrocyte formation and remyelination (Hubler et al., 2019, Nature 560 (7718), 372-376).

Myelin is lipid-based molecule which forms protective layers (myelin sheathes) around nerve cell axons and insulates the axons. Demyelinating diseases, or myelin-related diseases, are a result of these myelin sheathes being damaged, degraded, or reduced in thickness. The loss of the myelin sheathes disrupts the electronic signals from the brain and can lead to nerve damage, vision loss, numbness, muscle weakness, cognitive decline, loss of motor functions, and other similar symptoms. In some myelin-related diseases, such as multiple sclerosis, a subject's immune system targets and breaks down their own myelin sheathes. The ability to repair and regenerate the myelin sheathes is key to treating these myelin-related diseases. Due to its function converting 8-9 sterols, inhibition of EBP is a potential target for activating remyelination, as its inhibition leads to an increase of these 8-9 sterol starting materials (Theodoropoulous et al, 2020, J Am. Chem. Soc., 142, (13), 6128-6138).

In addition to its role in remyeliniation, EBP has also been shown to be a key enzyme in certain colorectal cancers due to the reduction in essential lipids such as cholesterol (Theodoropoulous et al, 2020, J Am. Chem. Soc., 142, (13), 6128-6138).

Thus, there is a need for EBP inhibitors as potential therapeutic agents for treating diseases or disorders that are responsive to EBP inhibition.

### SUMMARY OF THE INVENTION

The present disclosure provides compounds that are EBP inhibitors. In a first aspect, the present disclosure relates to compounds having the Formula I:

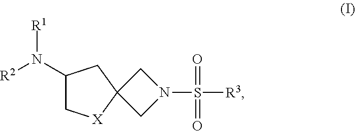

or a pharmaceutically acceptable salt thereof, wherein:
   X is O, CH₂, or a bond;
   R¹ and R² are each independently selected from H, C₁₋₆alkyl, C₁₋₃haloalkyl, C₃₋₈cycloalkyl, Het, and —Z-Het, wherein the C₁₋₆alkyl, C₃₋₈cycloalkyl, and Het are each optionally substituted with one or more R⁴, provided at least one of R¹ and R² is not H;
   or R¹ and R² together with the N atom from which they are attached form a 4 to 7-membered monocyclic heterocycle or 6 to 10-membered bicyclic heterocycle, each of which is optionally substituted with one or more R⁴;
   Z is C₁₋₄alkyl optionally substituted with one or more R⁴;
   Het is a 4 to 6 membered monocyclic heterocyclyl, or a 6 to 8-membered bicyclic heterocyclyl, each of which is optionally substituted with R⁴;
   each R⁴ is independently selected from OR^{4a}, halo, C₁₋₃alkyl, C₁₋₃haloalkyl, and 4 to 7-membered monocyclic heterocycle;
   R^{4a} is selected from H, C₁₋₃alkyl, and C₁₋₃haloalkyl;
   R³ is phenyl, 5 or 6-membered monocyclic heteroaryl, 9 or 10-membered bicyclic heteroaryl, or 6 to 10 membered bicyclic heterocycle, wherein the phenyl, 5 or 6-membered monocyclic heteroaryl, 9 or 10-membered bicyclic heteroaryl, and 6 to 10 membered bicyclic heterocycle are each optionally substituted with one or more substituent R⁵;
   each R⁵ is independently selected from C₁₋₃alkyl, C₁₋₃haloalkyl, OR^{5a}, cyano, and halo;
   R^{5a} is selected from H, C₁₋₃alkyl, and C₃₋₆cycloalkyl, wherein the C₁₋₃alkyl is optionally substituted with one or more halo;
   provided that R¹ and R² together with the N atom from which they are attached do not form an unsubstituted pyrrolidine, unsubstituted piperidine, or 4-methyl piperazine.

Another aspect of the disclosure relates to pharmaceutical compositions comprising compounds of Formula (I) or pharmaceutically acceptable salts thereof, and a pharmaceutical carrier.

In yet another aspect, the present disclosure provides a method of treating a disease or disorder that is responsive to inhibition of EBP in a subject comprising administering to said subject an effective amount of at least one compound described herein or a pharmaceutically acceptable salt thereof. In some embodiments, the present disclosure provides a method for treating multiple sclerosis. In some embodiments, the present disclosure provides a method for promoting myelination in a subject with a myelin-related disorder.

Another aspect of the present disclosure relates to the use of at least one compound described herein or a pharmaceutically acceptable salt thereof, for the manufacture of a medicament for the treatment of a disease or disorder responsive to inhibition of EBP. Also provided is a compound described herein or a pharmaceutically acceptable salt thereof for use in treating a disease or disorder responsive to inhibition of EBP.

### DETAILED DESCRIPTION OF THE INVENTION

The present disclosure provides compounds and pharmaceutical compositions thereof that may be useful in the treatment of diseases or disorders through mediation of EBP function/activity, such as multiple sclerosis or other myelin-related disorders. In some embodiments, the compounds of present disclosure are EBP inhibitors.

### Compounds and Compositions

In a first embodiment, the present disclosure provides a compound of Formula (I):

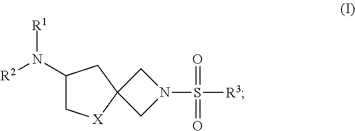

or a pharmaceutically acceptable salt thereof, wherein the variables in Formula (I) are as defined in the first aspect above.

In a second embodiment, for the compounds of Formula (I), or a pharmaceutically acceptable salt thereof, R³ is phenyl or 5 or 6-membered monocyclic heteroaryl, each of which are each optionally substituted with one to three substituent R⁵; and the remaining variables are as described in the first embodiment.

In a third embodiment, for the compounds of Formula (I), or a pharmaceutically acceptable salt thereof, R³ is phenyl, pyridinyl, pyrimidinyl, or pyrazoyl, each of which are each optionally substituted with one to three substituent R⁵; and the remaining variables are as described in the second embodiment.

In a fourth embodiment, for the compounds of Formula (I), or a pharmaceutically acceptable salt thereof, R³ is represented by the following formula:

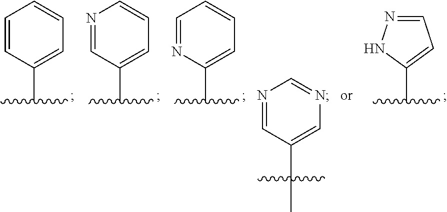

wherein each of the formula depicted above is optionally substituted with one to three R⁵; and the remaining variables are as described in the second embodiment.

In a fifth embodiment, for the compounds of Formula (I), or a pharmaceutically acceptable salt thereof, R¹ and R² together with the N atom from which they are attached form a saturated 4 to 6-membered monocyclic heterocycle or a saturated 7 to 10 membered bicyclic heterocycle, each of which is optionally substituted with one or two R⁴; and the remaining variables are as described in the first, second, third, or fourth embodiment.

In a sixth embodiment, for the compounds of Formula (I), or a pharmaceutically acceptable salt thereof, R¹ and R² together with the nitrogen atom from which they are attached form groups represented by the following formula:

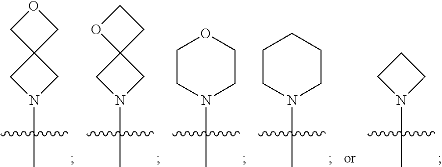

wherein each of the formula depicted above is optionally substituted with one or two R⁴; and the remaining variables are as described in the fifth embodiment.

In a seventh embodiment, for the compounds of Formula (I), or a pharmaceutically acceptable salt thereof, R¹ is H or C₁₋₃alkyl optionally substituted with one to three R⁴; R² is C₁₋₃alkyl, C₃₋₆cycloalkyl, Het, or —Z-Het, wherein the C₁₋₃alkyl, C₃₋₆cycloalkyl; and Het is a saturated 4 to 6 membered monocyclic heterocycle containing 1 to 2 heteroatoms selected from oxygen and nitrogen, at least one of which is oxygen, wherein the saturated 4 to 6 membered monocyclic heterocycle is optionally substituted with one to three R⁴; and the remaining variables are as described in the first, second, third, or fourth embodiment.

In an eighth embodiment, for the compounds of Formula (I), or a pharmaceutically acceptable salt thereof, Het is selected from tetraphydropyran and oxetane, each of which is optionally substituted with one to two R⁴; and the remaining variables are as described in the seventh embodiment.

In a ninth embodiment, the compound of the present disclosure is represented by Formula (II):

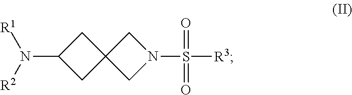

or a pharmaceutically acceptable salt thereof, wherein the variables in Formula (II) are as defined in the first, second, third, fourth, fifth, sixth, seventh, or eighth embodiment.

In a tenth embodiment, for the compounds of Formula (I) or (II), or a pharmaceutically acceptable salt thereof, R³ is represented by the following formula:

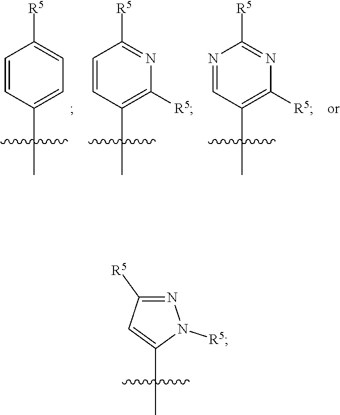

and the remaining variables are as described in the ninth embodiment.

In an eleventh embodiment, for the compounds of Formula (II), or a pharmaceutically acceptable salt thereof, R⁵, for each occurrence, is independently selected from C₁₋₃alkyl, C₁₋₃ haloalkyl, and OR^{5a}; and the remaining variables are as described in the ninth or tenth embodiment.

In a twelfth embodiment, for the compounds of Formula (II), or a pharmaceutically acceptable salt thereof, R⁵, for each occurrence is independently selected from —CH₃, —CF₃, —CF₂CH₃, —CH(CH₃)₂, and —OCHF₂; and the remaining variables are as described in the eleventh embodiment.

In a thirteenth embodiment, for the compounds of Formula (II), or a pharmaceutically acceptable salt thereof, R¹ and R² together with the nitrogen atom from which they are attached form groups represented by the following formula:

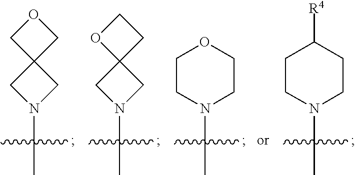

and the remaining variables are as described in the ninth, tenth, eleventh, or twelfth embodiment.

In a fourteenth embodiment, for the compounds of Formula (II), or a pharmaceutically acceptable salt thereof, R¹ is H and R² is represented by the following formula

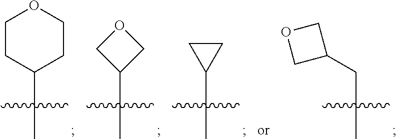

wherein each of the formula depicted above is optionally substituted with one or two R⁴; and the remaining variables are as described in the ninth, tenth, eleventh, or twelfth embodiment. In an alternative fourteenth embodiment, for the compounds of Formula (II), or a pharmaceutically acceptable salt thereof, R¹ is H and R² is represented by the following formula

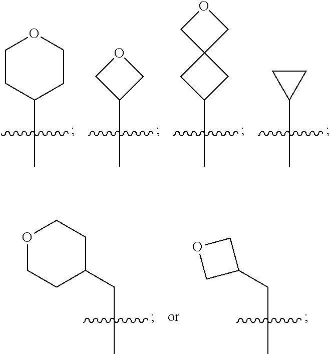

wherein each of the formula depicted above is optionally substituted with one or two R⁴; and the remaining variables are as described in the ninth, tenth, eleventh, or twelfth embodiment.

In a fifteenth embodiment, for the compounds of Formula (II), or a pharmaceutically acceptable salt thereof, R² is represented by the following formula

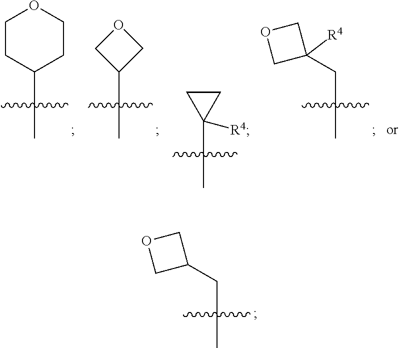

and the remaining variables are as described in the fourteenth embodiment. In an alternative fifteenth embodiment, for the compounds of Formula (II), or a pharmaceutically acceptable salt thereof, R² is represented by the following formula

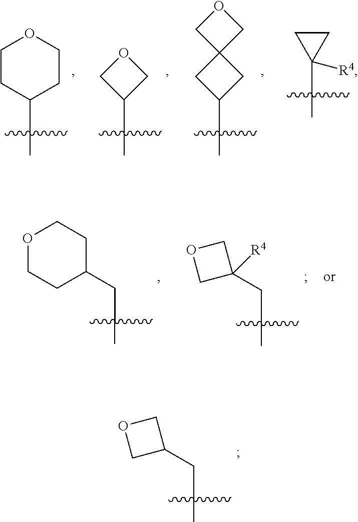

and the remaining variables are as described in the fourteenth embodiment.

In a sixteenth embodiment, for the compounds of Formula (II), or a pharmaceutically acceptable salt thereof, R⁴, for each occurrence, is independently selected from C₁₋₃alkyl and 4 to 7-membered monocyclic heterocycle; and the remaining variables are as described in the ninth, tenth, eleventh, twelfth, thirteenth, fourteenth, or fifteenth embodiment.

In a seventeenth embodiment, for the compounds of Formula (II), or a pharmaceutically acceptable salt thereof, R⁴, for each occurrence, is independently selected from —CH₃ and tetrahydropyran; and the remaining variables are as described in the sixteenth embodiment.

In an eighteenth embodiment, the compound of the present disclosure is represented by Formula (III) or (IV):

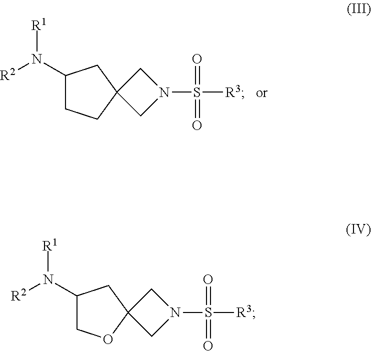

or a pharmaceutically acceptable salt thereof, wherein the variables in Formula (III) or (IV) are as defined in the first, second, third, fourth, fifth, sixth, seventh, or eighth embodiment.

In a nineteenth embodiment, the compound of the present disclosure is represented by Formula (IIIA), (IIIB), (IVA), or (IVB):

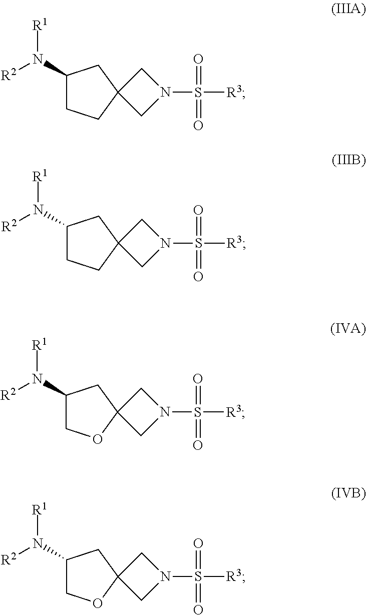

or a pharmaceutically acceptable salt thereof, wherein the variables in Formula (IIIA), (IIIB), (IVA), or (IVB) are as defined in the eighteenth embodiment.

In a twentieth embodiment, for the compounds of Formula (III), (IIIA), (IIIB), (IV), (IVA), or (IVB), or a pharmaceutically acceptable salt thereof, R³ is represented by the following formula:

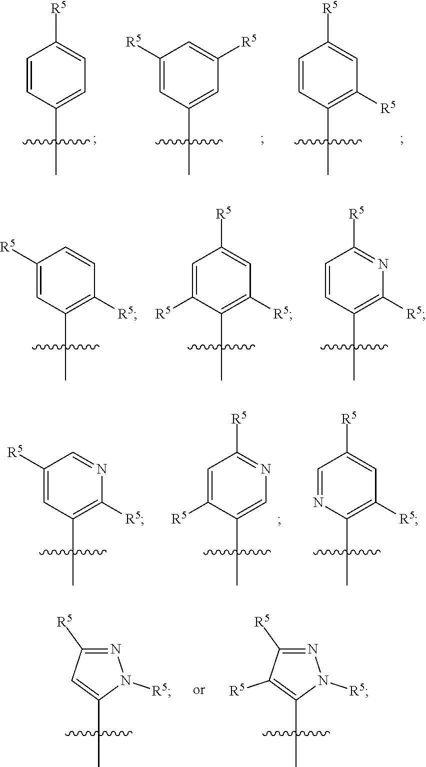

and the remaining variables are as described in the eighteenth or nineteenth embodiment.

In a twenty-first embodiment, for the compounds of Formula (III), (IIIA), (IIIB), (IV), (IVA), or (IVB), or a pharmaceutically acceptable salt thereof, R⁵, for each occurrence, is independently selected from halo, cyano, C₁₋₂alkyl, C₁₋₂haloalkyl and OR a; and R^{5a} is C₁₋₂alkyl or C₁₋₂haloalkyl; and the remaining variables are as described in the eighteenth, nineteenth, or twentieth embodiment.

In a twenty-second embodiment, for the compounds of Formula (III), (IIIA), (IIIB), (IV), (IVA), or (IVB), or a pharmaceutically acceptable salt thereof, R⁵, for each occurrence, is independently selected from F, Cl, cyano, —CH₃, —CF₃, —CHF₂, —OCH₃, and —OCHF₂; and the remaining variables are as described in the twenty-first embodiment.

In a twenty-third embodiment, for the compounds of Formula (III), (IIIA), (IIIB), (IV), (IVA), or (IVB), or a pharmaceutically acceptable salt thereof, R¹ and R² together with the nitrogen atom from which they are attached form groups represented by the following formula:

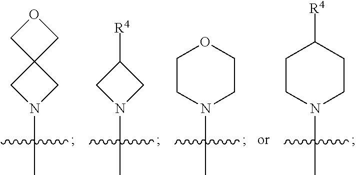

and the remaining variables are as described in the eighteenth, nineteenth, twentieth, twenty-first, or twenty-second embodiment.

In a twenty-fourth embodiment, for the compounds of Formula (III), (IIIA), (IIIB), (IV), (IVA), or (IVB), or a pharmaceutically acceptable salt thereof, R¹ is H or C₁₋₃alkyl; and R² is C₁₋₃alkyl, C₃₋₆cycloalkyl, —Z-Het, or Het, each of which is optionally substituted with one to three R⁴; and the remaining variables are as described in the eighteenth, nineteenth, twentieth, twenty-first, or twenty-second embodiment.

In a twenty-fifth embodiment, for the compounds of Formula (III), (IIIA), (IIIB), (IV), (IVA), or (IVB), or a pharmaceutically acceptable salt thereof, R¹ is H or —CH₃ and R² is C₁₋₃alkyl optionally substituted with one R⁴, or is represented by the following formula:

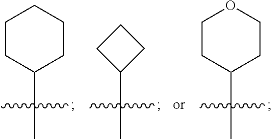

wherein each of the formula depicted above is optionally substituted with one or two R⁴; and the remaining variables are as described in the twenty-fourth embodiment.

In a twenty-sixth embodiment, for the compounds of Formula (III), (IIIA), (IIIB), (IV), (IVA), or (IVB), or a pharmaceutically acceptable salt thereof, R¹ is H or —CH₃ and R² is represented by the following formula:

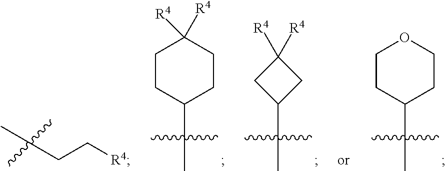

and the remaining variables are as described in the twenty-fifth embodiment.

In a twenty-seventh embodiment, for the compounds of Formula (III), (IIIA), (IIIB), (IV), (IVA), or (IVB), or a pharmaceutically acceptable salt thereof, R⁴, for each occurrence, is independently selected from OR^{4a}, halo, and C₁₋₃alkyl, and wherein R^{4a} is C₁₋₃alkyl; and the remaining variables are as described in the eighteenth, nineteenth, twentieth, twenty-first, twenty-second, twenty-third, twenty-fourth, twenty-fifth, or twenty-sixth embodiment.

In a twenty-eighth embodiment, for the compounds of Formula (III), (IIIA), (IIIB), (IV), (IVA), or (IVB), or a pharmaceutically acceptable salt thereof, R⁴, for each occurrence, is independently selected from halo, —CH₃, and —OCH₃; and the remaining variables are as described in the twenty-seventh embodiment.

In a twenty-ninth embodiment, the present disclosure provides a compound described herein (e.g., a compound of any one of Examples 1 to 83), or a pharmaceutically acceptable salt thereof.

In a thirtieth embodiment, the present disclosure provides a compound selected from the group consisting of:
6-(2-((6-(1,1-Difluoroethyl)-2-methylpyridin-3-yl)sulfonyl)-2-azaspiro[3.3]heptan-6-yl)-2-oxa-6-azaspiro[3.3]heptane;
2-((6-(1,1-Difluoroethyl)-2-methylpyridin-3-yl)sulfonyl)-N-(tetrahydro-2H-pyran-4-yl)-2-azaspiro[3.3]heptan-6-amine;
2-((2-Methyl-6-(trifluoromethyl)pyridin-3-yl)sulfonyl)-N-(tetrahydro-2H-pyran-4-yl)-2-azaspiro[3.3]heptan-6-amine;
6-(2-((2-Methyl-6-(trifluoromethyl)pyridin-3-yl)sulfonyl)-2-azaspiro[3.3]heptan-6-yl)-2-oxa-6-azaspiro[3.3]heptane;
4-(2-((2-Methyl-6-(trifluoromethyl)pyridin-3-yl)sulfonyl)-2-azaspiro[3.3]heptan-6-yl)morpholine;
6-(2-((2-Methyl-6-(trifluoromethyl)pyridin-3-yl)sulfonyl)-2-azaspiro[3.3]heptan-6-yl)-1-oxa-6-azaspiro[3.3]heptane;
2-((2-Methyl-6-(trifluoromethyl)pyridin-3-yl)sulfonyl)-N-(1-(tetrahydro-2H-pyran-4-yl)cyclopropyl)-2-azaspiro[3.3]heptan-6-amine;
2-((2-Methyl-6-(trifluoromethyl)pyridin-3-yl)sulfonyl)-N-(oxetan-3-yl)-2-azaspiro[3.3]heptan-6-amine;
2-((2-Methyl-6-(trifluoromethyl)pyridin-3-yl)sulfonyl)-N-((3-methyloxetan-3-yl)methyl)-2-azaspiro[3.3]heptan-6-amine;
4-(2-((1-Methyl-3-(trifluoromethyl)-1H-pyrazol-5-yl)sulfonyl)-2-azaspiro[3.3]heptan-6-yl)morpholine;
2-((1-Methyl-3-(trifluoromethyl)-1H-pyrazol-5-yl)sulfonyl)-N-(oxetan-3-ylmethyl)-2-azaspiro[3.3]heptan-6-amine;
2-((1-Methyl-3-(trifluoromethyl)-1H-pyrazol-5-yl)sulfonyl)-N-((3-methyloxetan-3-yl)methyl)-2-azaspiro[3.3]heptan-6-amine;
2-((1-Methyl-3-(trifluoromethyl)-1H-pyrazol-5-yl)sulfonyl)-N-(oxetan-3-yl)-2-azaspiro[3.3]heptan-6-amine;
6-(2-((1-Methyl-3-(trifluoromethyl)-1H-pyrazol-5-yl)sulfonyl)-2-azaspiro[3.3]heptan-6-yl)-2-oxa-6-azaspiro[3.3]heptane;
6-(2-((1-Methyl-3-(trifluoromethyl)-1H-pyrazol-5-yl)sulfonyl)-2-azaspiro[3.3]heptan-6-yl)-1-oxa-6-azaspiro[3.3]heptane;
6-(2-((4-Methyl-2-(trifluoromethyl)pyrimidin-5-yl)sulfonyl)-2-azaspiro[3.3]heptan-6-yl)-1-oxa-6-azaspiro[3.3]heptane;
6-(2-((4-Methyl-2-(trifluoromethyl)pyrimidin-5-yl)sulfonyl)-2-azaspiro[3.3]heptan-6-yl)-2-oxa-6-azaspiro[3.3]heptane;
2-Methyl-N-(2-(tetrahydro-2H-pyran-4-yl)-2-azaspiro[3.3]heptan-6-yl)-6-(trifluoromethyl)pyridine-3-sulfonamide;
4-(2-((3-Isopropyl-1-methyl-1H-pyrazol-5-yl)sulfonyl)-2-azaspiro[3.3]heptan-6-yl)morpholine;
2-((4-(Difluoromethoxy)phenyl)sulfonyl)-6-(4-methylpiperidin-1-yl)-2-azaspiro[3.3]heptane;
2-((2-Methoxy-5-methylpyridin-3-yl)sulfonyl)-N-(tetrahydro-2H-pyran-4-yl)-5-oxa-2-azaspiro[3.4]octan-7-amine;
2-((2-Methoxy-5-methylpyridin-3-yl)sulfonyl)-7-(2-oxa-6-azaspiro[3.3]heptan-6-yl)-5-oxa-2-azaspiro[3.4]octane;
2-((2-Methoxy-5-methylpyridin-3-yl)sulfonyl)-7-(3-methoxyazetidin-1-yl)-5-oxa-2-azaspiro[3.4]octane;
N-(4,4-Difluorocyclohexyl)-2-((2-methoxy-5-methylpyridin-3-yl)sulfonyl)-5-oxa-2-azaspiro[3.4]octan-7-amine;
N-(3,3-Difluorocyclobutyl)-2-((2-methoxy-5-methylpyridin-3-yl)sulfonyl)-5-oxa-2-azaspiro[3.4]octan-7-amine;
3-((7-((4,4-Difluorocyclohexyl)amino)-5-oxa-2-azaspiro[3.4]octan-2-yl)sulfonyl)-4-fluorobenzonitrile;
4-Fluoro-3-((7-((tetrahydro-2H-pyran-4-yl)amino)-5-oxa-2-azaspiro[3.4]octan-2-yl)sulfonyl)benzonitrile;
3-((7-(2-Oxa-6-azaspiro[3.3]heptan-6-yl)-5-oxa-2-azaspiro[3.4]octan-2-yl)sulfonyl)-5-fluorobenzonitrile;
4-(2-((3,5-Dimethylpyridin-2-yl)sulfonyl)-2-azaspiro[3.4]octan-6-yl)morpholine;
6-(2-((6-Methoxy-2-methylpyridin-3-yl)sulfonyl)-2-azaspiro[3.4]octan-6-yl)-2-oxa-6-azaspiro[3.3]heptane;
N-(4,4-Difluorocyclohexyl)-2-((6-methoxy-2-methylpyridin-3-yl)sulfonyl)-2-azaspiro[3.4]octan-6-amine;
2-((6-Methoxy-2-methylpyridin-3-yl)sulfonyl)-N-(2-methoxyethyl)-N-methyl-2-azaspiro[3.4]octan-6-amine;
N-(2-Methoxyethyl)-N-methyl-2-((1-methyl-3-(trifluoromethyl)-1H-pyrazol-5-yl)sulfonyl)-2-azaspiro[3.4]octan-6-amine;
4-(2-((3-Methoxy-1-methyl-1H-pyrazol-5-yl)sulfonyl)-2-azaspiro[3.4]octan-6-yl)morpholine;
4-(2-((1-Methyl-3-(trifluoromethyl)-1H-pyrazol-5-yl)sulfonyl)-2-azaspiro[3.4]octan-6-yl)morpholine;
4-(2-((1,3-Dimethyl-1H-pyrazol-5-yl)sulfonyl)-2-azaspiro[3.4]octan-6-yl)morpholine;
4-(2-((4-Chloro-1,3-dimethyl-1H-pyrazol-5-yl)sulfonyl)-2-azaspiro[3.4]octan-6-yl)morpholine;
4-(2-((4-(Difluoromethyl)-1,3-dimethyl-1H-pyrazol-5-yl)sulfonyl)-2-azaspiro[3.4]octan-6-yl)morpholine;
4-(2-((6-Methoxy-2-(trifluoromethyl)pyridin-3-yl)sulfonyl)-2-azaspiro[3.4]octan-6-yl)morpholine;
4-(2-((2-Methyl-6-(trifluoromethyl)pyridin-3-yl)sulfonyl)-2-azaspiro[3.4]octan-6-yl)morpholine;
4-(2-((4-Methyl-6-(trifluoromethyl)pyridin-3-yl)sulfonyl)-2-azaspiro[3.4]octan-6-yl)morpholine;
4-(2-((6-Chloro-2-methylpyridin-3-yl)sulfonyl)-2-azaspiro[3.4]octan-6-yl)morpholine;
4-(2-(Mesitylsulfonyl)-2-azaspiro[3.4]octan-6-yl)morpholine;
4-(2-((4-(Difluoromethoxy)phenyl)sulfonyl)-2-azaspiro[3.4]octan-6-yl)morpholine;
4-(2-((2,4-Dimethylphenyl)sulfonyl)-2-azaspiro[3.4]octan-6-yl)morpholine;
4-(2-((2,4-Difluorophenyl)sulfonyl)-2-azaspiro[3.4]octan-6-yl)morpholine;
4-(2-((4,6-Dimethylpyridin-3-yl)sulfonyl)-2-azaspiro[3.4]octan-6-yl)morpholine;
4-(2-((6-methoxy-2-methylpyridin-3-yl)sulfonyl)-2-azaspiro[3.4]octan-6-yl)morpholine;
4-(2-((1-Methyl-3-(trifluoromethyl)-1H-pyrazol-5-yl)sulfonyl)-2-azaspiro[3.4]octan-6-yl)morpholine;
(R)-4-(2-((1-Methyl-3-(trifluoromethyl)-1H-pyrazol-5-yl)sulfonyl)-2-azaspiro[3.4]octan-6-yl)morpholine;
3-Fluoro-5-((6-morpholino-2-azaspiro[3.4]octan-2-yl)sulfonyl)benzonitrile;
(R)-3-Fluoro-5-((6-morpholino-2-azaspiro[3.4]octan-2-yl)sulfonyl)benzonitrile;
4-(2-((1-methyl-3-(trifluoromethyl)-1H-pyrazol-5-yl)sulfonyl)-2-azaspiro[3.4]octan-6-yl)morpholine;
(S)-4-(2-((1-methyl-3-(trifluoromethyl)-1H-pyrazol-5-yl)sulfonyl)-2-azaspiro[3.4]octan-6-yl)morpholine;
3-Fluoro-5-((6-morpholino-2-azaspiro[3.4]octan-2-yl)sulfonyl)benzonitrile;
(S)-3-Fluoro-5-((6-morpholino-2-azaspiro[3.4]octan-2-yl)sulfonyl)benzonitrile;
2-(Mesitylsulfonyl)-7-morpholino-5-oxa-2-azaspiro[3.4]octane;
2-((4-(Difluoromethoxy)phenyl)sulfonyl)-7-(4-methylpiperidin-1-yl)-5-oxa-2-azaspiro[3.4]octane;
2-((1,3-Dimethyl-1H-pyrazol-5-yl)sulfonyl)-7-(4-methylpiperidin-1-yl)-5-oxa-2-azaspiro[3.4]octane;
2-((6-Methoxy-2-methylpyridin-3-yl)sulfonyl)-7-(4-methylpiperidin-1-yl)-5-oxa-2-azaspiro[3.4]octane;
2-((1-methyl-3-(trifluoromethyl)-1H-pyrazol-5-yl)sulfonyl)-7-morpholino-5-oxa-2-azaspiro[3.4]octane;
(R)-2-((1-methyl-3-(trifluoromethyl)-1H-pyrazol-5-yl)sulfonyl)-7-morpholino-5-oxa-2-azaspiro[3.4]octane;
2-((2-Methyl-6-(trifluoromethyl)pyridin-3-yl)sulfonyl)-7-morpholino-5-oxa-2-azaspiro[3.4]octane;
(R)-2-((2-Methyl-6-(trifluoromethyl)pyridin-3-yl)sulfonyl)-7-morpholino-5-oxa-2-azaspiro[3.4]octane;
3-((7-((4,4-Difluorocyclohexyl)(methyl)amino)-5-oxa-2-azaspiro[3.4]octan-2-yl)sulfonyl)-4-fluorobenzonitrile;
4-(2-((2-Methyl-6-(trifluoromethyl)pyridin-3-yl)sulfonyl)-2-azaspiro[3.4]octan-6-yl)morpholine;
(R)-4-(2-((2-Methyl-6-(trifluoromethyl)pyridin-3-yl)sulfonyl)-2-azaspiro[3.4]octan-6-yl)morpholine;
(S)-4-(2-((2-methyl-6-(trifluoromethyl)pyridin-3-yl)sulfonyl)-2-azaspiro[3.4]octan-6-yl)morpholine;
4-(2-((4-Methyl-6-(trifluoromethyl)pyridin-3-yl)sulfonyl)-2-azaspiro[3.4]octan-6-yl)morpholine;
(R)-4-(2-((4-Methyl-6-(trifluoromethyl)pyridin-3-yl)sulfonyl)-2-azaspiro[3.4]octan-6-yl)morpholine;
(S)-4-(2-((4-methyl-6-(trifluoromethyl)pyridin-3-yl)sulfonyl)-2-azaspiro[3.4]octan-6-yl)morpholine;
4-(2-((6-Chloro-2-methylpyridin-3-yl)sulfonyl)-2-azaspiro[3.4]octan-6-yl)morpholine;
(R)-4-(2-((6-Chloro-2-methylpyridin-3-yl)sulfonyl)-2-azaspiro[3.4]octan-6-yl)morpholine;
(S)-4-(2-((6-chloro-2-methylpyridin-3-yl)sulfonyl)-2-azaspiro[3.4]octan-6-yl)morpholine;
4-(2-((1,3-dimethyl-1H-pyrazol-5-yl)sulfonyl)-2-azaspiro[3.4]octan-6-yl)morpholine;
(R)-4-(2-((1,3-dimethyl-1H-pyrazol-5-yl)sulfonyl)-2-azaspiro[3.4]octan-6-yl)morpholine;
(S)-4-(2-((1,3-dimethyl-1H-pyrazol-5-yl)sulfonyl)-2-azaspiro[3.4]octan-6-yl)morpholine;
6-(2-((6-(1,1-difluoroethyl)-2-methylpyridin-3-yl)sulfonyl)-2-azaspiro[3.3]heptan-6-yl)-1-oxa-6-azaspiro[3.3]heptane;
2-((6-(1,1-difluoroethyl)-2-methylpyridin-3-yl)sulfonyl)-N-(oxetan-3-yl)-2-azaspiro[3.3]heptan-6-amine;
2-((6-(1,1-difluoroethyl)-2-methylpyridin-3-yl)sulfonyl)-N-(2-oxaspiro[3.3]heptan-6-yl)-2-azaspiro[3.3]heptan-6-amine;
2-((6-(1,1-difluoroethyl)-2-methylpyridin-3-yl)sulfonyl)-N-((tetrahydro-2H-pyran-4-yl)methyl)-2-azaspiro[3.3]heptan-6-amine;
2-((6-(1,1-difluoroethyl)-2-methylpyridin-3-yl)sulfonyl)-N-methyl-N-(tetrahydro-2H-pyran-4-yl)-2-azaspiro[3.3]heptan-6-amine;
2-((6-(1,1-difluoroethyl)-2-methylpyridin-3-yl)sulfonyl)-N-methyl-N-(oxetan-3-yl)-2-azaspiro[3.3]heptan-6-amine;
2-((6-(1,1-difluoroethyl)-2-methylpyridin-3-yl)sulfonyl)-N-methyl-N-(2-oxaspiro[3.3]heptan-6-yl)-2-azaspiro[3.3]heptan-6-amine;
2-((6-(1,1-difluoroethyl)-2-methylpyridin-3-yl)sulfonyl)-N-methyl-N-((tetrahydro-2H-pyran-4-yl)methyl)-2-azaspiro[3.3]heptan-6-amine;
2-((4-methyl-2-(trifluoromethyl)pyrimidin-5-yl)sulfonyl)-N-(tetrahydro-2H-pyran-4-yl)-2-azaspiro[3.3]heptan-6-amine;
2-((4-methyl-2-(trifluoromethyl)pyrimidin-5-yl)sulfonyl)-N-(oxetan-3-yl)-2-azaspiro[3.3]heptan-6-amine;
2-((4-methyl-2-(trifluoromethyl)pyrimidin-5-yl)sulfonyl)-N-(2-oxaspiro[3.3]heptan-6-yl)-2-azaspiro[3.3]heptan-6-amine;
2-((4-methyl-2-(trifluoromethyl)pyrimidin-5-yl)sulfonyl)-N-((tetrahydro-2H-pyran-4-yl)methyl)-2-azaspiro[3.3]heptan-6-amine;
N-methyl-2-((4-methyl-2-(trifluoromethyl)pyrimidin-5-yl)sulfonyl)-N-(tetrahydro-2H-pyran-4-yl)-2-azaspiro[3.3]heptan-6-amine;
N-methyl-2-((4-methyl-2-(trifluoromethyl)pyrimidin-5-yl)sulfonyl)-N-(oxetan-3-yl)-2-azaspiro[3.3]heptan-6-amine;
N-methyl-2-((4-methyl-2-(trifluoromethyl)pyrimidin-5-yl)sulfonyl)-N-(2-oxaspiro[3.3]heptan-6-yl)-2-azaspiro[3.3]heptan-6-amine; and
N-methyl-2-((4-methyl-2-(trifluoromethyl)pyrimidin-5-yl)sulfonyl)-N-((tetrahydro-2H-pyran-4-yl)methyl)-2-azaspiro[3.3]heptan-6-amine;
   or a or a pharmaceutically acceptable salt thereof.

In a thirty-first embodiment, the present disclosure provides a pharmaceutical composition comprising a compound according to any one of the preceding embodiments, or a pharmaceutically acceptable salt thereof.

In a thirty-second embodiment, the present disclosure provides a method of treating a disease or disorder mediated by EBP comprising administering to a subject an effective amount of a compound according to any one of embodiments one to thirty, or a pharmaceutically acceptable salt thereof, or the pharmaceutical composition of the thirty-first embodiment.

In a thirty-third embodiment, the present disclosure provides a compound according to any one of embodiments one to thirty, for use in the treatment of a disease or disorder mediated by EBP.

In a thirty-fourth embodiment, the present disclosure provides the use of a compound according to any one of embodiments one to thirty in the manufacture of a medicament for the treatment of a disease or disorder mediated by EBP.

The compounds and intermediates described herein may be isolated and used as the compound per se. Alternatively, when a moiety is present that is capable of forming a salt, the compound or intermediate may be isolated and used as its corresponding salt. As used herein, the terms “salt” or “salts” refers to an acid addition or base addition salt of a compound described herein. “Salts” include in particular “pharmaceutical acceptable salts”. The term “pharmaceutically acceptable salts” refers to salts that retain the biological effectiveness and properties of the compounds described herein and, which typically are not biologically or otherwise undesirable. In many cases, the compounds of the present disclosure are capable of forming acid and/or base salts by virtue of the presence of amino and/or carboxyl groups or groups similar thereto.

Pharmaceutically acceptable acid addition salts can be formed with inorganic acids or organic acids, e.g., acetate, aspartate, benzoate, besylate, bromide/hydrobromide, bicarbonate/carbonate, bisulfate/sulfate, camphorsulfornate, chloride/hydrochloride, chlortheophyllonate, citrate, ethandisulfonate, fumarate, gluceptate, gluconate, glucuronate, hippurate, hydroiodide/iodide, isethionate, lactate, lactobionate, laurylsulfate, malate, maleate, malonate, mandelate, mesylate, methylsulphate, naphthoate, napsylate, nicotinate, nitrate, octadecanoate, oleate, oxalate, palmitate, pamoate, phosphate/hydrogen phosphate/dihydrogen phosphate, polygalacturonate, propionate, stearate, succinate, sulfate, sulfosalicylate, tartrate, tosylate and trifluoroacetate salts.

Inorganic acids from which salts can be derived include, for example, hydrochloric acid, hydrobromic acid, sulfuric acid, nitric acid, phosphoric acid, and the like.

Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like.

Pharmaceutically acceptable base addition salts can be formed with inorganic and organic bases.

Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts.

Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.

The salts can be synthesized by conventional chemical methods from a compound containing a basic or acidic moiety. Generally, such salts can be prepared by reacting free acid forms of these compounds with a stoichiometric amount of the appropriate base (such as Na, Ca, Mg, or K hydroxide, carbonate, bicarbonate or the like), or by reacting free base forms of these compounds with a stoichiometric amount of the appropriate acid. Such reactions are typically carried out in water or in an organic solvent, or in a mixture of the two. Generally, use of non-aqueous media like ether, ethyl acetate, ethanol, isopropanol, or acetonitrile is desirable, where practicable. Lists of additional suitable salts can be found, e.g., in “Remington's Pharmaceutical Sciences”, 20th ed., Mack Publishing Company, Easton, Pa., (1985); and in “Handbook of Pharmaceutical Salts: Properties, Selection, and Use” by Stahl and Wermuth (Wiley-VCH, Weinheim, Germany, 2002).

Isotopically-labeled compounds of Formula (I) can generally be prepared by conventional techniques known to those skilled in the art or by processes analogous to those described in the accompanying Examples and Preparations using an appropriate isotopically-labeled reagents in place of the non-labeled reagent previously employed. In one embodiment, the present disclosure provides deuterated compounds described herein or a pharmaceutically acceptable salt thereof.

Pharmaceutically acceptable solvates in accordance with the invention include those wherein the solvent of crystallization may be isotopically substituted, e.g. D₂O, d₆-acetone, d₆-DMSO.

It will be recognized by those skilled in the art that the compounds of the present invention may contain chiral centers and as such may exist in different stereoisomeric forms. As used herein, the term “an optical isomer” or “a stereoisomer” refers to any of the various stereo isomeric configurations which may exist for a given compound of the present disclosure. It is understood that a substituent may be attached at a chiral center of a carbon atom. Therefore, the disclosure includes enantiomers, diastereomers or racemates of the compound.

“Enantiomers” are a pair of stereoisomers that are non-superimposable mirror images of each other. A 1:1 mixture of a pair of enantiomers is a “racemic” mixture. The term “racemic” or “rac” is used to designate a racemic mixture where appropriate. When designating the stereochemistry for the compounds of the present invention, a single stereoisomer with known relative and absolute configuration of the two chiral centers is designated using the conventional RS system (e.g., (1S,2S)). “Diastereoisomers” are stereoisomers that have at least two asymmetric atoms, but which are not mirror-images of each other. The absolute stereochemistry is specified according to the Cahn-Ingold-Prelog R—S system. When a compound is a pure enantiomer the stereochemistry at each chiral carbon may be specified by either R or S. Resolved compounds whose absolute configuration is unknown can be designated (+) or (−) depending on the direction (dextro- or levorotatory) which they rotate plane polarized light at the wavelength of the sodium D line. Alternatively, the resolved compounds can be defined by the respective retention times for the corresponding enantiomers/diastereomers via chiral HPLC.

Certain of the compounds described herein contain one or more asymmetric centers or axes and may thus give rise to enantiomers, diastereomers, and other stereoisomeric forms that may be defined, in terms of absolute stereochemistry, as (R)- or (S)-.

Unless specified otherwise, the compounds of the present disclosure are meant to include all such possible stereoisomers, including racemic mixtures, optically pure forms and intermediate mixtures. Optically active (R)- and (S)-stereoisomers may be prepared using chiral synthons or chiral reagents, or resolved using conventional techniques (e.g., separated on chiral SFC or HPLC chromatography columns, such as CHIRALPAK® and CHIRALCEL® available from DAICEL Corp. using the appropriate solvent or mixture of solvents to achieve good separation). If the compound contains a double bond, the substituent may be E or Z configuration. If the compound contains a disubstituted cycloalkyl, the cycloalkyl substituent may have a cis- or trans-configuration. All tautomeric forms are also intended to be included.

### Methods of Use

The compounds disclosed herein have EBP inhibitory activity. As used herein, “EBP inhibitory activity” refers to the ability of a compound or composition to induce a detectable decrease in EBP activity in vivo or in vitro (e.g., at least 10% decrease in EBP activity as measured by a given assay such as the bioassay described in the examples and known in the art).

In certain embodiments, the present disclosure provides a method of treating a disease or disorder responsive to inhibition of EBP activity (referred herein as “EBP mediated disease or disorder” or “disease or disorder mediated by EBP”) in a subject in need of the treatment. The method comprises administering to the subject a compound described herein (e.g., a compound described in any one of the first to thirty embodiments) or a pharmaceutically acceptable salt thereof or a pharmaceutical composition thereof.

In certain embodiments, the present disclosure provides the use of a compound described herein (e.g., a compound described in any one of the first to thirtieth embodiments) or a pharmaceutically acceptable salt thereof or a pharmaceutical composition comprising a compound described herein or a pharmaceutically acceptable salt thereof for the manufacture of a medicament for the treatment of a EBP mediated disorder or disease in a subject in need of the treatment.

In certain embodiments, the present disclosure provides a compound described herein (e.g., a compound described in any one of the first to thirtieth embodiments) or a pharmaceutically acceptable salt thereof or a pharmaceutical composition comprising a compound described herein or a pharmaceutically acceptable salt thereof for use in the treatment of a EBP mediated disorder or disease in a subject in need of the treatment.

In certain embodiments, the EBP mediated disorder is colorectal cancer.

In certain embodiments, the present disclosure provides a method of treating an autoimmune disease in a subject in need of the treatment. The method comprises administering to the subject a compound described herein (e.g., a compound described in any one of the first to thirtieth embodiments) or a pharmaceutically acceptable salt thereof or a pharmaceutical composition thereof.

In certain embodiments, the present disclosure provides the use of a compound described herein (e.g., a compound described in any one of the first to thirtieth embodiments) or a pharmaceutically acceptable salt thereof or a pharmaceutical composition comprising a compound described herein or a pharmaceutically acceptable salt thereof for the manufacture of a medicament for the treatment of an autoimmune disease in a subject in need of the treatment.

In certain embodiments, the present disclosure provides a compound described herein (e.g., a compound described in any one of the first to thirtieth embodiments) or a pharmaceutically acceptable salt thereof or a pharmaceutical composition comprising a compound described herein or a pharmaceutically acceptable salt thereof for use in the treatment of an autoimmune disease in a subject in need of the treatment.

In certain embodiments, the autoimmune disease is multiple sclerosis (MS). The compounds of the present disclosure can be used for treating all stages of MS, including relapsing multiple sclerosis (or relapsing form(s) of multiple sclerosis), relapsing-remitting multiple sclerosis, primary progress multiple sclerosis, secondary progressive multiple sclerosis and clinically isolated syndrome (hereinafter “CIS”).

Relapsing multiple sclerosis (or relapsing form(s) of multiple sclerosis) includes clinically isolated syndrome, relapsing-remitting multiple sclerosis and active secondary progressive multiple sclerosis.

Relapsing-remitting multiple sclerosis is a stage of MS characterized by unpredictable relapses followed by periods of months to years of relative quiet (remission) with no new signs of disease activity. Deficits that occur during attacks may either resolve or leave problems, the latter in about 40% of attacks and being more common the longer a person has had the disease. This describes the initial course of 80% of individuals with multiple sclerosis.

Secondary progressive multiple sclerosis occurs in around 65% of those with initial relapsing-remitting multiple sclerosis, who eventually have progressive neurologic decline between acute attacks without any definite periods of remission. Occasional relapses and minor remissions may appear. The most common length of time between disease onset and conversion from relapsing-remitting to secondary progressive multiple sclerosis is 19 years.

Primary progressive multiple sclerosis is characterized by the same symptoms of secondary progressive multiple sclerosis, i.e., progressive neurologic decline between acute attacks without any definite periods of remission, without the prior relapsing-remitting stage.

CIS is a first episode of neurologic symptoms caused by inflammation and demyelination in the central nervous system. The episode, which by definition must last for at least 24 hours, is characteristic of multiple sclerosis but does not yet meet the criteria for a diagnosis of MS because people who experience a CIS may or may not go on to develop MS. When CIS is accompanied by lesions on a brain MRI (magnetic resonance imaging) that are similar to those seen in MS, the person has a high likelihood of a second episode of neurologic symptoms and diagnosis of relapsing-remitting MS. When CIS is not accompanied by MS-like lesions on a brain MRI, the person has a much lower likelihood of developing MS.

In certain embodiments, the present disclosure provides a method of promoting myelination in a subject with a myelin-related disease or disorder in a subject in need of the treatment. The method comprises administering to the subject a compound described herein (e.g., a compound described in any one of the first to thirtieth embodiments) or a pharmaceutically acceptable salt thereof or a pharmaceutical composition thereof.

In certain embodiments, the present disclosure provides the use of a compound described herein (e.g., a compound described in any one of the first to thirtieth embodiments) or a pharmaceutically acceptable salt thereof or a pharmaceutical composition comprising a compound described herein or a pharmaceutically acceptable salt thereof for the manufacture of a medicament for promoting myelination in a subject with a myelin-related disease or disorder in a subject in need of the treatment.

In certain embodiments, the present disclosure provides a compound described herein (e.g., a compound described in any one of the first to thirtieth embodiments) or a pharmaceutically acceptable salt thereof or a pharmaceutical composition comprising a compound described herein or a pharmaceutically acceptable salt thereof for use in promoting myelination in a subject with a myelin-related disease or disorder in a subject in need of the treatment.

In certain embodiments, the myelin-related disease or disorder is selected from multiple sclerosis (MS), neuromyelitis optica (NMO), optic neuritis, pediatric leukodystrophies, neonatal white matter injury, age-related dementia, schizophrenia, progressive multifocal leukoencephalopathy (PML), encephalomyelitis (EPL), acute disseminated encephalomyelitis (ADEM), central pontine myelolysis (CPM), adrenoleukodystrophy, Alexander's disease, Pelizaeus Merzbacher disease (PMD), Vanishing White Matter Disease, Wallerian Degeneration, transverse myelitis, amylotrophic lateral sclerosis (ALS), Huntington's disease, Alzheimer's disease, Parkinson's disease, spinal cord injury, traumatic brain injury, post radiation injury, neurologic complications of chemotherapy, stroke, acute ischemic optic neuropathy, vitamin E deficiency, isolated vitamin E deficiency syndrome, Bassen-Kornzweig syndrome, Marchiafava-Bignami syndrome, autism, metachromatic leukodystrophy, trigeminal neuralgia, acute disseminated encephalitis, chronic inflammatory demyelinating polyneuropathy, Guillian-Barre syndrome, Charcot-Marie-Tooth disease, Bell's palsy and radiation-induced demyelination, for example, neuromyelitis optica (NMO), optic neuritis, pediatric leukodystrophies, neonatal white matter injury, age-related dementia, and schizophrenia.

In certain embodiments, the present disclosure provides a method of treating cancer in a subject in need of the treatment. The method comprises administering to the subject a compound described herein (e.g., a compound described in any one of the first to thirtieth embodiments) or a pharmaceutically acceptable salt thereof or a pharmaceutical composition thereof.

In certain embodiments, the present disclosure provides the use of a compound described herein (e.g., a compound described in any one of the first to thirtieth embodiments) or a pharmaceutically acceptable salt thereof or a pharmaceutical composition comprising a compound described herein or a pharmaceutically acceptable salt thereof for the manufacture of a medicament for the treatment of cancer in a subject in need of the treatment.

In certain embodiments, the present disclosure provides a compound described herein (e.g., a compound described in any one of the first to thirtieth embodiments) or a pharmaceutically acceptable salt thereof or a pharmaceutical composition comprising a compound described herein or a pharmaceutically acceptable salt thereof for use in treating cancer in a subject in need of the treatment.

In certain embodiments, the cancer is colorectal cancer.

In certain embodiments, the present disclosure relates to the aforementioned methods, wherein said subject is a mammal. In certain embodiments, the subject is a primate. In certain embodiments, the subject is a human.

As used herein, an “effective amount” and a “therapeutically effective amount” can used interchangeably. It means an amount effective for treating or lessening the severity of one or more of the diseases, disorders or conditions as recited herein. In some embodiments, the effective dose can be between 10 g and 500 mg.

The compounds and compositions, according to the methods of the present disclosure, may be administered using any amount and any route of administration effective for treating or lessening the severity of one or more of the diseases, disorders or conditions recited above.

In certain embodiments, the present disclosure relates to the aforementioned methods, wherein said compound is administered parenterally. In certain embodiments, the present disclosure relates to the aforementioned methods, wherein said compound is administered intramuscularly, intravenously, subcutaneously, orally, pulmonary, rectally, intrathecally, topically or intranasally. In certain embodiments, the present disclosure relates to the aforementioned methods, wherein said compound is administered systemically.

The compounds of the present invention can be used as a pharmaceutical composition (e.g., a compound of the present invention and at least one pharmaceutically acceptable carrier). As used herein, the term “pharmaceutically acceptable carrier” includes generally recognized as safe (GRAS) solvents, dispersion media, surfactants, antioxidants, preservatives (e.g., antibacterial agents, antifungal agents), isotonic agents, salts, preservatives, drug stabilizers, buffering agents (e.g., maleic acid, tartaric acid, lactic acid, citric acid, acetic acid, sodium bicarbonate, sodium phosphate, and the like), and the like and combinations thereof, as would be known to those skilled in the art (see, for example, Remington's Pharmaceutical Sciences, 18th Ed. Mack Printing Company, 1990, pp. 1289-1329). Except insofar as any conventional carrier is incompatible with the active ingredient, its use in the therapeutic or pharmaceutical compositions is contemplated. For purposes of this disclosure, solvates and hydrates are considered pharmaceutical compositions comprising a compound of the present invention and a solvent (i.e., solvate) or water (i.e., hydrate).

The formulations may be prepared using conventional dissolution and mixing procedures. For example, the bulk drug substance (i.e., compound of the present invention or stabilized form of the compound (e.g., complex with a cyclodextrin derivative or other known complexation agent)) is dissolved in a suitable solvent in the presence of one or more of the excipients described above. The compound of the present invention is typically formulated into pharmaceutical dosage forms to provide an easily controllable dosage of the drug and to give the patient an elegant and easily handleable product.

The pharmaceutical composition (or formulation) for application may be packaged in a variety of ways depending upon the method used for administering the drug. Generally, an article for distribution includes a container having deposited therein the pharmaceutical formulation in an appropriate form. Suitable containers are well-known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

The pharmaceutical composition comprising a compound of the present disclosure is generally formulated for use as a parenteral or oral administration or alternatively suppositories.

For example, the pharmaceutical oral compositions of the present disclosure can be made up in a solid form (including without limitation capsules, tablets, pills, granules, powders or suppositories), or in a liquid form (including without limitation solutions, suspensions or emulsions). The pharmaceutical compositions can be subjected to conventional pharmaceutical operations such as sterilization and/or can contain conventional inert diluents, lubricating agents, or buffering agents, as well as adjuvants, such as preservatives, stabilizers, wetting agents, emulsifiers and buffers, etc.

Typically, the pharmaceutical compositions are tablets or gelatin capsules comprising the active ingredient together with
a) diluents, e.g., lactose, dextrose, sucrose, mannitol, sorbitol, cellulose and/or glycine;
b) lubricants, e.g., silica, talcum, stearic acid, its magnesium or calcium salt and/or polyethylene glycol; for tablets also
c) binders, e.g., magnesium aluminum silicate, starch paste, gelatin, tragacanth, methylcellulose, sodium carboxymethylcellulose and/or polyvinylpyrrolidone; if desired
d) disintegrants, e.g., starches, agar, alginic acid or its sodium salt, or effervescent mixtures; and/or
e) absorbents, colorants, flavors and sweeteners.

Tablets may be either film coated or enteric coated according to methods known in the art.

Suitable compositions for oral administration include a compound of the disclosure in the form of tablets, lozenges, aqueous or oily suspensions, dispersible powders or granules, emulsion, hard or soft capsules, or syrups or elixirs. Compositions intended for oral use are prepared according to any method known in the art for the manufacture of pharmaceutical compositions and such compositions can contain one or more agents selected from the group consisting of sweetening agents, flavoring agents, coloring agents and preserving agents in order to provide pharmaceutically elegant and palatable preparations. Tablets may contain the active ingredient in admixture with nontoxic pharmaceutically acceptable excipients which are suitable for the manufacture of tablets. These excipients are, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets are uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

The parenteral compositions (e.g, intravenous (IV) formulation) are aqueous isotonic solutions or suspensions. The parenteral compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. The compositions are generally prepared according to conventional mixing, granulating or coating methods, respectively, and contain about 0.1-75%, or contain about 1-50%, of the active ingredient.

The compound of the present disclosure or pharmaceutical composition thereof for use in a subject (e.g., human) is typically administered orally or parenterally at a therapeutic dose. When administered intravenously via infusion, the dosage may depend upon the infusion rate at which an IV formulation is administered. In general, the therapeutically effective dosage of a compound, the pharmaceutical composition, or the combinations thereof, is dependent on the species of the subject, the body weight, age and individual condition, the disorder or disease or the severity thereof being treated. A physician, pharmacist, clinician or veterinarian of ordinary skill can readily determine the effective amount of each of the active ingredients necessary to prevent, treat or inhibit the progress of the disorder or disease.

The above-cited dosage properties are demonstrable in vitro and in vivo tests using advantageously mammals, e.g., mice, rats, dogs, monkeys or isolated organs, tissues and preparations thereof. The compounds of the present invention can be applied in vitro in the form of solutions, e.g., aqueous solutions, and in vivo either enterally, parenterally, advantageously intravenously, e.g., as a suspension or in aqueous solution. The dosage in vitro may range between about 10-3 molar and 10-9 molar concentrations.

### DEFINITIONS

As used herein, a “patient,” “subject” or “individual” are used interchangeably and refer to either a human or non-human animal. The term includes mammals such as humans. Typically, the animal is a mammal. A subject also refers to for example, primates (e.g., humans, male or female), cows, sheep, goats, horses, dogs, cats, rabbits, rats, mice, fish, birds and the like. In certain embodiments, the subject is a primate. Preferably, the subject is a human.

As used herein, the term “inhibit”, “inhibition” or “inhibiting” refers to the reduction or suppression of a given condition, symptom, or disorder, or disease, or a significant decrease in the baseline activity of a biological activity or process.

As used herein, the term “treat”, “treating” or “treatment” of any disease, condition or disorder, refers to the management and care of a patient for the purpose of combating the disease, condition, or disorder and includes the administration of a compound of the present invention to obtaining desired pharmacological and/or physiological effect. The effect can be therapeutic, which includes achieving, partially or substantially, one or more of the following results: partially or totally reducing the extent of the disease, condition or disorder; ameliorating or improving a clinical symptom, complications or indicator associated with the disease, condition or disorder; or delaying, inhibiting or decreasing the likelihood of the progression of the disease, condition or disorder; or eliminating the disease, condition or disorder. In certain embodiments, the effect can be to prevent the onset of the symptoms or complications of the disease, condition or disorder.

As used herein, the term “cancer” has the meaning normally accepted in the art. The term can broadly refer to abnormal cell growth.

As used herein, the term “autoimmune disease” has the meaning normally accepted the art. The term can broadly refer to a disease where the host's immune system targets or attacks normal or healthy tissue of the host.

As used herein, the term “myelination” has the meaning normally accepted in the art. The term can broadly mean the process by which myelin is produced.

As used herein, the term “myelin-related disease or disorder”, “demyelinating disorder”, or “demyelation disorder” has the meaning normally accepted in the art. These terms can broadly refer to diseases or disorders which involve damage to myelin.

As used herein, a subject is “in need of” a treatment if such subject would benefit biologically, medically or in quality of life from such treatment (preferably, a human).

As used herein, the phrase “optionally substituted” is used interchangeably with the phrase “substituted or unsubstituted.” In general the term “optionally substituted” refers to the replacement of hydrogen radicals in a given structure with the radical of a specified substituent. Specific substituents are described in the definitions and in the description of compounds and examples thereof. Unless otherwise indicated, an optionally substituted group can have a substituent at each substitutable position of the group, and when more than one position in any given structure can be substituted with more than one substituent selected from a specified group, the substituent can be either the same or different at every position. In some embodiments, an optionally substituted group can be substituted with one or more substituents, each of which can the same or different. In some embodiments, the “one or more” substituents can be 1, 2, 3, 4, 5, 6, etc. substituents, each of which can the same or different. In some embodiment, the “one or more” substituents can be 1 to 6, 1 to 4, 1 to 3 or 1 to 2 substituents, each of which can the same or different.

As used herein, the term “alkyl” refers to a fully saturated branched or unbranched hydrocarbon moiety. The term “C₁₋₄alkyl” refers to an alkyl having 1 to 4 carbon atoms. The terms “C₁₋₃alkyl” and “C₁₋₂alkyl” are to be construed accordingly. Representative examples of “C₁₋₄alkyl” include, but are not limited to, methyl, ethyl, n-propyl, iso-propyl, n-butyl, sec-butyl, iso-butyl, and tert-butyl. Similarly, the alkyl portion (i.e., alkyl moiety) of an alkoxy have the same definition as above. When indicated as being “optionally substituted”, the alkane radical or alkyl moiety may be unsubstituted or substituted with one or more substituents (generally, one to three substituents except in the case of halogen substituents such as perchloro or perfluoroalkyls).

As used herein, the term “alkoxy” refers to a fully saturated branched or unbranched alkyl moiety attached through an oxygen bridge (i.e. a —O— C₁₋₄ alkyl group wherein C₁₋₄ alkyl is as defined herein). Representative examples of alkoxy include, but are not limited to, methoxy, ethoxy, propoxy, 2-propoxy, butoxy, tert-butoxy and the like. Preferably, alkoxy groups have about 1-4 carbons, more preferably about 1-2 carbons. The term “C₁₋₂ alkoxy” is to be construed accordingly.

As used herein, the term “C₁₋₄ alkoxyC₁₋₄ alkyl” refers to a C₁₋₄alkyl group as defined herein, wherein at least of the hydrogen atoms is replaced by an C₁₋₄alkoxy. The C₁₋₄alkoxyC₁₋₄ alkyl group is connected through the rest of the molecule described herein through the alkyl group.

The number of carbon atoms in a group is specified herein by the prefix “Cₓ₋ₓₓ”, wherein x and xx are integers. For example, “C₁₋₃ alkyl” is an alkyl group which has from 1 to 3 carbon atoms.

“Halogen” or “halo” may be fluorine, chlorine, bromine or iodine.

As used herein, the term “halo-substituted-C₁₋₄alkyl” or “C₁₋₄haloalkyl” refers to a C₁₋₄ alkyl group as defined herein, wherein at least one of the hydrogen atoms is replaced by a halo atom. The C₁₋₄haloalkyl group can be monohalo-C₁₋₄alkyl, dihalo-C₁₋₄alkyl or polyhalo-C₁₋₄ alkyl including perhalo-C₁₋₄alkyl. A monohalo-C₁₋₄alkyl can have one iodo, bromo, chloro or fluoro within the alkyl group. Dihalo-C₁₋₄alkyl and polyhalo-C₁₋₄alkyl groups can have two or more of the same halo atoms or a combination of different halo groups within the alkyl. Typically the polyhalo-C₁₋₄alkyl group contains up to 9, or 8, or 7, or 6, or 5, or 4, or 3, or 2 halo groups. Non-limiting examples of C₁₋₄haloalkyl include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. A perhalo-C₁₋₄alkyl group refers to a C₁₋₄alkyl group having all hydrogen atoms replaced with halo atoms.

The term “aryl” refers to an aromatic carbocyclic single ring or two fused ring system containing 6 to 10 carbon atoms. Examples include phenyl and naphthyl.

The term “heteroaryl” refers to a 5- to 12-membered aromatic radical containing 1-4 heteroatoms selected from N, O, and S. In some instances, nitrogen atoms in a heteroaryl may be quaternized. The term “heteroaryl” may be used interchangeably with the terms “heteroaryl ring”, “heteroaryl group”, or “heteroaromatic”. A heteroaryl group may be mono- or bi-cyclic. Monocyclic heteroaryl includes, for example, pyrazolyl, imidazolyl, oxazolyl, pyridinyl, furanyl, oxadiazolyl, thiophenyl, and the like. Bi-cyclic heteroaryls include groups in which a monocyclic heteroaryl ring is fused to one or more aryl or heteroaryl rings. Non-limiting examples include pyrazolopyridinyl, pyrazolopyridinyl, benzotriazolyl, imidazopyridinyl, and indoyl.

The term “carbocyclic ring” or “carbocyclyl” refers to a 4- to 12-membered saturated or partially unsaturated hydrocarbon ring and may exist as a single ring, bicyclic ring (including fused, spiral or bridged carbocyclic rings) or a spiral ring. Bi-cyclic carbocyclyl groups include, e.g., unsaturated carbocyclic radicals fused to another unsaturated carbocyclic radical, cycloalkyl, or aryl, such as, for example, 2,3-dihydroindenyl, decahydronaphthalenyl, and 1,2,3,4-tetrahydronaphthalenyl. Unless specified otherwise, the carbocyclic ring generally contains 4- to 10-ring members.

The term “C₃₋₆ cycloalkyl” refers to a carbocyclic ring which is fully saturated (e.g., cyclopropyl, cyclobutyl, cyclopentyl, and cyclohexyl).

The term “heterocycle” or “heterocyclyl” refers to a 4- to 12-membered saturated or partially unsaturated heterocyclic ring containing 1 to 4 heteroatoms independently selected from N, O, and S. A heterocyclyl group may be mono- or bicyclic (e.g., a bridged, fused, or spiro bicyclic ring). Examples of monocyclic saturated or partially unsaturated heterocyclic radicals include, without limitation, oxetanyl, tetrahydrofuranyl, tetrahydropyranyl, and piperdinyl. Bi-cyclic heterocyclyl groups include, e.g., unsaturated heterocyclic radicals fused to another unsaturated heterocyclic radical, cycloalkyl, aryl, or heteroaryl ring, such as, for example, tetrahydro-3H-[1,2,3]triazolo[4,5-c]pyridinyl, 2-oxa-6-azaspiro[3.3]heptanyl, 5-oxabicyclo[2.1.1]hexanyl and 9-azabicyclo[3.3.1]nonanyl. In some embodiments, the heterocyclyl group is a 4 to 6 membered monocyclic heterocyclyl group. In some embodiments, the heterocyclyl group is a 4 to 6 membered monocyclic saturated heterocyclyl group. In some embodiments, the heterocyclyl group is a 8 to 10 membered bicyclic heterocyclyl group. In some embodiments, the heterocyclyl group is a 8 to 10 membered bicyclic saturated heterocyclyl group.

As used herein the term “spiral” ring means a two-ring system wherein both rings share one common atom. Examples of spiral rings include, 2-oxa-6-azaspiro[3.3]heptanyl and the like.

The term “fused” ring refers to two ring systems share two adjacent ring atoms. Fused heterocycles have at least one the ring systems contain a ring atom that is a heteroatom selected from O, N and S (e.g., 3-oxabicyclo[3.1.0]hexane).

As used herein the term “bridged” refers to a 5 to 10 membered cyclic moiety connected at two non-adjacent ring atoms (e.g. 5-oxabicyclo[2.1.1]hexane).

The phrase “pharmaceutically acceptable” indicates that the substance, composition or dosage form must be compatible chemically and/or toxicologically, with the other ingredients comprising a formulation, and/or the mammal being treated therewith.

Unless specified otherwise, the term “compounds of the present disclosure” refers to compounds of Formula (I), as well as all stereoisomers (including diastereoisomers and enantiomers), rotamers, tautomers, isotopically labeled compounds (including deuterium substitutions). When a moiety is present that is capable of forming a salt, then salts are included as well, in particular pharmaceutically acceptable salts.

As used herein, the term “a,” “an,” “the” and similar terms used in the context of the present invention (especially in the context of the claims) are to be construed to cover both the singular and plural unless otherwise indicated herein or clearly contradicted by the context. The use of any and all examples, or exemplary language (e.g., “such as”) provided herein is intended merely to better illuminate the invention and does not pose a limitation on the scope of the invention otherwise claimed.

It is also possible that the intermediates and compounds of the present invention may exist in different tautomeric forms, and all such forms are embraced within the scope of the invention. The term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerizations. A specific example of a proton tautomer is the imidazole moiety where the proton may migrate between the two ring nitrogens. Valence tautomers include interconversions by reorganization of some of the bonding electrons.

In one embodiment, the present disclosure relates to a compound of the Formula (I) as defined herein, in free form. In another embodiment, the present disclosure relates to a compound of the Formula (I) as defined herein, in salt form. In another embodiment, the present disclosure relates to a compound of the Formula (I) as defined herein, in acid addition salt form. In a further embodiment, the present disclosure relates to a compound of the Formula (I) as defined herein, in pharmaceutically acceptable salt form. In yet a further embodiment, the present disclosure relates to a compound of the Formula (I) as defined herein, in pharmaceutically acceptable acid addition salt form. In yet a further embodiment, the present disclosure relates to any one of the compounds of the Examples in free form. In yet a further embodiment, the present disclosure relates to any one of the compounds of the Examples in salt form. In yet a further embodiment, the present disclosure relates to any one of the compounds of the Examples in acid addition salt form. In yet a further embodiment, the present disclosure relates to any one of the compounds of the Examples in pharmaceutically acceptable salt form. In still another embodiment, the present disclosure relates to any one of the compounds of the Examples in pharmaceutically acceptable acid addition salt form.

Compounds of the present disclosure may be synthesized by synthetic routes that include processes analogous to those well-known in the chemical arts, particularly in light of the description contained herein. The starting materials are generally available from commercial sources such as Sigma-Aldrich or are readily prepared using methods well known to those skilled in the art (e.g., prepared by methods generally described in Louis F. Fieser and Mary Fieser, Reagents for Organic Synthesis, v. 1-19, Wiley, New York (1967-1999 ed.), or Beilsteins Handbuch der organischen Chemie, 4, Aufl. ed. Springer-Verlag, Berlin, including supplements (also available via the Beilstein online database)).

For illustrative purposes, the reaction schemes depicted below provide potential routes for synthesizing the compounds of the present disclosure as well as key intermediates. For a more detailed description of the individual reaction steps, see the Examples section below. Although specific starting materials and reagents are depicted in the schemes and discussed below, other starting materials and reagents can be easily substituted to provide a variety of derivatives and/or reaction conditions.

### EXEMPLIFICATION

### Abbreviations

PE=petroleum ether
EtOAc=EA=ethyl acetate
ESI=electrospray ionisation
MeOH=methanol
EtOH=ethanol
DCE=1,2-dichloroethane
DCM=dichloromethane
CHCl₃=chloroform
HCl=hydrochloric acid
H₂O=water
IPA=isopropyl alcohol
LCMS=liquid chromatography mass spectrometry
HFIP=hexafluoro-2-propanol
HPLC=high pressure liquid chromatography
THF=tetrahydrofuran
MeCN=ACN=acetonitrile
MgSO₄=magnesium sultate
DMSO=dimetylsulfoxide
AcOH=acetic acid
TFA=trifluoroacetic acid
DIPEA=diisopropylethyl amine
N₂=Nitrogen
NH₄HCO₃=Ammonium Bicarbonate
t-BuOH=tert-butanol
NH₄Cl=ammonium chloride
NaH=sodium hydride
Na₂SO₄=sodium sulfate
K₂CO₃=potassium carbonate
NaHCO₃=sodium bicarbonate
NaBH(OAc)₃=STAB=sodium triacetoxyborohydride
SiO₂=silicon dioxide or silica
PDA=Photo Diode Array Detection
TosMIC=toluenesulfonylmethyl isocyanide
TLC=Thin Layer Chromatography
LiHMDS=Lithium bis(trimethylsilyl)amide

### GENERAL METHODS

### LCMS Instrumentation Specifications:

Agilent Technologies 1200 Series LC/MSD system: DAD\ELSD Alltech 3300 and Agilent LC\MSD G6130A, G6120B mass-spectrometer.
Agilent Technologies 1260 Infinity LC/MSD system: DAD\ELSD Alltech 3300 and Agilent LC\MSD G6120B mass-spectrometer.
Agilent Technologies 1260 Infinity II LC/MSD system: DAD\ELSD G7102A 1290 Infinity II and Agilent LC\MSD G6120B mass-spectrometer.
Agilent 1260 Series LC/MSD system: DAD\ELSD and Agilent LC\MSD (G6120B) mass-spectrometer.
UHPLC Agilent 1290 Series LC/MSD system: DAD\ELSD and Agilent LC\MSD (G6125B) mass-spectrometer.

### HPLC Analytical Method Specifications:

Column: Agilent Poroshell 120 SB-C18 4.6×30 mm 2.7 μm, with UHPLC Guard Infinity Lab Poroshell 120 SB-C18 4.6×5 mm 2.7 μm
Column Temperature, 60 C
Injection volume 0.5 μL
Modifier: Formic acid 0.1% (v/v) conc.
Method: 99% water/1% MeCN (initial conditions), HOLD initial conditions for 0.1 min, linear gradient to 0% water/100% MeCN at 1.5 min, HOLD 0% water/100% MeCN to 1.73 min, linear gradient to 99% water/1% MeCN at 1.74 min. Flow rate, 3.0 mL/min.
UV scan: 207-223 nm, 246-262 nm, 272-288 nm

### QC Analysis LC/MS Method Conditions:

### Ammonium Hydroxide (Basic pH) Conditions

MS mode: MS:ESI+ scan range 165-650 daltons
PDA: 200-400 nm scan range
Column: Waters ACQUITY UPLC BEH C18 2.1×50 mm, 1.7 μm; Part No. 186002350
Modifier: Ammonium hydroxide 0.2% (v/v) conc.
Method: 95% water/5% MeCN (initial conditions) linear gradient to 5% water/95% MeCN at 3.75 min, HOLD 5% water/95% MeCN to 4 min. Flow rate, 0.8 mL/min.

### Trifluoroacetic Acid (Acidic pH) Conditions

MS mode: MS:ESI+ scan range 165-650 daltons
PDA: 200-400 nm scan range
Column: Waters ACQUITY UPLC BEH C18 2.1×50 mm, 1.7 μm; Part No. 186002350
Modifier: Trifluoroacetic acid 0.1% (v/v) conc.
Method: 95% water/5% MeCN (initial conditions) linear gradient to 5% water/95% MeCN at 3.75 min, HOLD 5% water/95% MeCN to 4 min. Flow rate, 0.8 mL/min.

### General Prep HPLC Conditions:

### Ammonium Hydroxide (Basic pH) Conditions

Flow rate: 30 mL/min
MS mode: MS:ESI+ scan range 165-650 daltons
PDA: 200-400 nm scan range
Column: Waters XSELECT CSH C18 PREP 19×100 mm, 5 μm; Part No. 186005421
Modifier: 0.2% Ammonium hydroxide (v/v) conc.
Method: A % water/B % MeCN (initial conditions) linear gradient to A % water/B % MeCN at 8 min, ramp to 5% water/95% MeCN at 8.5 min, HOLD 5% water/95% MeCN to 10 min.
Flow rate: 50 mL/min
MS mode: MS:ESI+ scan range 165-650 daltons
PDA: 200-400 nm scan range
Column: Waters XSELECT CSH C18 PREP 30×100 mm, 5 μm; Part No. 186005425
Modifier: 0.2% NH₄OH (v/v) conc.
Method: A % water/B % MeCN (initial conditions) linear gradient to A % water/B % MeCN at 8 min, ramp to 5% water/95% MeCN at 8.5 min, HOLD 5% water/95% MeCN to 10 min.
Flow rate, 60 mL/min
MS mode: MS:ESI+ scan range 165-650 daltons
PDA: 200-400 nm scan range
Column: Waters XSELECT CSH C18 PREP 30×50 mm, 5 μm; Part No. 186005423 Modifier: 0.2% NH₄OH (v/v) conc.
Method: A % water/B % MeCN (initial conditions) linear gradient to A % water/B % MeCN at 8 min, ramp to 5% water/95% MeCN at 8.5 min, HOLD 5% water/95% MeCN to 10 min.

### Trifluoroacetic Acid (Acidic pH) Conditions

Flow rate, 30 mL/min
MS mode: MS:ESI+ scan range 165-650 daltons
PDA: 200-400 nm scan range
Column: Waters Sunfire OBD C18 PREP 19×100 mm, 5 μm; Part No. 186002567 Modifier: 0.1% Trifluoroacetic acid (v/v) conc.
Method: A % water/B % MeCN (initial conditions) linear gradient to A % water/B % MeCN at 8 min, ramp to 5% water/95% MeCN at 8.5 min, HOLD 5% water/95% MeCN to 10 min.
Flow rate, 50 mL/min
MS mode: MS:ESI+ scan range 165-650 daltons
PDA: 200-400 nm scan range
Column: Waters Sunfire OBD C18 PREP 30×100 mm, 5 μm; Part No. 186002572 Modifier: 0.1% Trifluoroacetic acid (v/v) conc.
Method: A % water/B % MeCN (initial conditions) linear gradient to A % water/B % MeCN at 8 min, ramp to 5% water/95% MeCN at 8.5 min, HOLD 5% water/95% MeCN to 10 min.
Flow rate, 60 mL/min
MS mode: MS:ESI+ scan range 165-650 daltons
PDA: 200-400 nm scan range
Column: Waters Sunfire OBD C18 PREP 30×50 mm, 5 μm; Part No. 186002570
Modifier: 0.1% Trifluoroacetic acid (v/v) conc.
Method: A % water/B % MeCN (initial conditions) linear gradient to A % water/B % MeCN at 8 min, ramp to 5% water/95% MeCN at 8.5 min, HOLD 5% water/95% MeCN to 10 min.

### EXPERIMENTAL PROCEDURES

### Example 1

### 6-(2-((6-(1,1-Difluoroethyl)-2-methylpyridin-3-yl)sulfonyl)-2-azaspiro[3.3]heptan-6-yl)-2-oxa-6-azaspiro[3.3]heptane

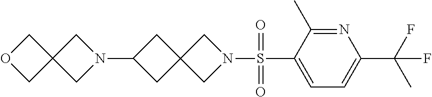 [SMILES: Cc1nc(C(C)(F)F)ccc1S(=O)(=O)N1CC2(CC(N3CC4(COC4)C3)C2)C1]

### 1. Synthesis of 1-(5-bromo-6-methylpyridin-2-yl)ethan-1-one

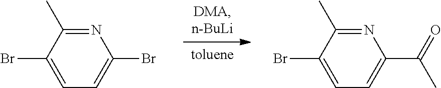

To a solution of 3,6-dibromo-2-methylpyridine (10 g, 39.9 mmol) in toluene (200 mL) was added n-BuLi (2.5 M, 17.5 mL) at −78 °C under a N₂ atmosphere and the solution stirred for 1 h. DMA (10.4 g, 119.6 mmol) was added, the mixture was stirred at −78 °C for 1 h and slowly warmed to 20 °C. The reaction was poured into saturated aqueous NH₄Cl solution, extracted with EtOAc and the combined organic extracts concentrated in vacuo. The extract was purified by silica gel column chromatography (PE/EtOAc=I/O to 8/1) to give 1-(5-bromo-6-methylpyridin-2-yl)ethan-1-one (8.1 g, 95%) as a yellow solid. ¹H NMR (400 MHz, CDCl₃): δ ppm 7.92 (d, J=8.0 Hz, 1H), 7.72 (d, J=8.0 Hz, 1H), 2.72 (s, 3H), 2.69 (s, 3H).

### 2. Synthesis of 3-bromo-6-(1,1-difluoroethyl)-2-methylpyridine

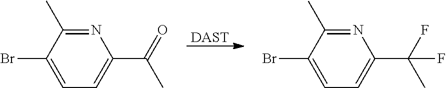

A solution of 1-(5-bromo-6-methylpyridin-2-yl)ethan-1-one (8.1 g, 37.8 mmol) in DAST (30 mL, 227.1 mmol) was stirred at 60 °C for 4 h. The reaction was quenched with saturated NaHCO₃ aq. until pH=7 and was extracted with DCM (100 mL×3). The combined organic layer was washed with brine (100 mL), dried over Na₂SO₄ and filtered. The filtrate was concentrated in vacuo and the residue was purified by CombiFlash® (PE/EtOAc=20/1 to 10/1) to give 3-bromo-6-(1,1-difluoroethyl)-2-methylpyridine (4.5 g, 50%) as colorless oil. LCMS m/z=236.0 [M+H]⁺.

### 3. Synthesis of 3-(benzylthio)-6-(1,1-difluoroethyl)-2-methylpyridine

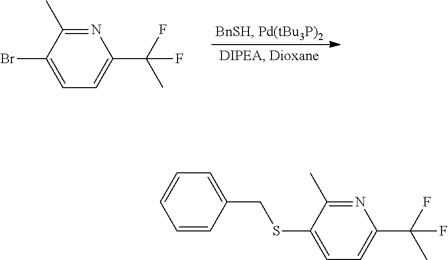

To a solution of 3-bromo-6-(1,1-difluoroethyl)-2-methylpyridine (2.2 g, 9.3 mmol) in dioxane (20 mL) was added DIPEA (4.9 mL, 28.0 mmol), Pd(tBu₃P)₂ (476 mg, 0.9 mmol) and BnSH (2.3 mL, 19.7 mmol) and the mixture was stirred at 100 °C for 16 h under N₂. The mixture was quenched with H₂O (100 mL) and extracted with DCM (50 mL×5). The combined organic phases were washed with brine (50 mL), dried over Na₂SO₄, filtered and concentrated to give 3-(benzylthio)-6-(1,1-difluoroethyl)-2-methylpyridine (4 g, crude) as yellow oil. LCMS m/z=280.1 [M+H]⁺.

### 4. Synthesis of 6-(1,1-difluoroethyl)-2-methylpyridine-3-sulfonyl chloride

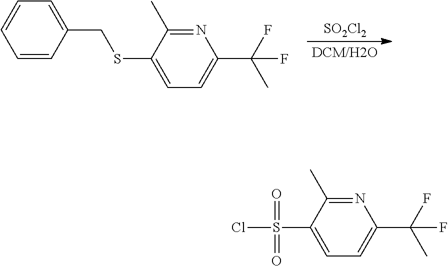 [SMILES: Cc1nc(C(C)(F)F)ccc1SCc1ccccc1 ; "SO2Cl2" ; "DCM/H2O" ; Cc1nc(C(C)(F)F)ccc1S(=O)(=O)Cl]

To a solution of 3-(benzylthio)-6-(1,1-difluoroethyl)-2-methylpyridine (1 g, 3.6 mmol) in DCM (10 mL) and water (2 mL), was added SO₂Cl₂ (2.1 mL, 25.1 mmol) at 0 °C and the reaction was stirred at 0 °C for 1 h under N₂. The mixture was diluted with water (50 mL) and extracted with DCM (50 mL×5). The combined organic phase was dried over anhydrous sodium sulfate, and concentrated in vacuo to give 2-((6-(1,1-difluoroethyl)-2-methylpyridin-3-yl)sulfonyl)-2-azaspiro[3.3]heptan-6-one (650 mg, crude) as yellow oil. ¹H NMR (400 MHz, CDCl₃): δ ppm 8.44 (d, J=8.4 Hz, 1H), 7.74 (d, J=8.4 Hz, 1H), 3.05 (s, 3H), 2.05 (t, J=18.8 Hz, 3H).

### 5. Synthesis of 2-((6-(1,1-difluoroethyl)-2-methylpyridin-3-yl)sulfonyl)-2-azaspiro[3.3]heptan-6-one

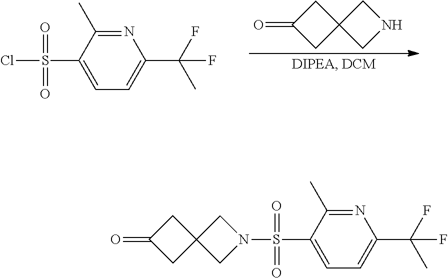

To a solution of 2-azaspiro[3.3]heptan-6-one TFA salt (530 mg, 2.4 mmol) in DCM (10 mL) was added DIPEA (6820 μL, 4.7 mmol), followed by 2-((6-(1,1-difluoroethyl)-2-methylpyridin-3-yl)sulfonyl)-2-azaspiro[3.3]heptan-6-one (605 mg, 2.36 mmol) in DCM (2 mL). The mixture was stirred at 20 °C for 1 h then diluted with water (30 mL) and extracted with DCM (30 mL×3). The combined organic phase was washed with water (20 mL), dried over anhydrous sodium sulfate, filtered and concentrated in vacuo. The crude was purified by column chromatography (0% to 6% MeOH in DCM) to give 2-((6-(1,1-difluoroethyl)-2-methylpyridin-3-yl)sulfonyl)-2-azaspiro[3.3]heptan-6-one (260 mg, 33%) as yellow solid. LCMS m/z=331.1 [M+H]⁺.

### 6. Synthesis of 6-(2-((6-(1,1-difluoroethyl)-2-methylpyridin-3-yl)sulfonyl)-2-azaspiro[3.3]heptan-6-yl)-2-oxa-6-azaspiro[3.3]heptane

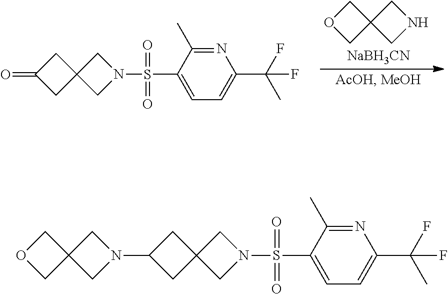

A solution of 2-((6-(1,1-difluoroethyl)-2-methylpyridin-3-yl)sulfonyl)-2-azaspiro[3.3]heptan-6-one (80 mg, 0.2 mmol) and 2-oxa-6-azaspiro[3.3]heptane oxalate (68 mg, 0.4 mmol) in MeOH (3 mL) was adjusted to pH 5-6 using AcOH and the solution stirred for 0.5 h at 20 °C. NaBH₃CN (76 mg, 1.2 mmol) was added and the mixture was stirred for 1 h at 20 °C. The mixture was purified by prep-HPLC (Welch Xtimate C18 150×25 mm×5 μm, Condition: water (NH₄HCO₃)-MeCN, 28% to 48%, Flow Rate (mL/min): 25) to give 6-(2-((6-(1,1-difluoroethyl)-2-methylpyridin-3-yl)sulfonyl)-2-azaspiro[3.3]heptan-6-yl)-2-oxa-6-azaspiro[3.3]heptane (32 mg, 32%) as white solid. LCMS m/z=414.1 [M+H]⁺. ¹H NMR (400 MHz, MeOH-d₄): δ ppm 8.34 (d, J=8.0 Hz, 1H), 7.70 (d, J=8.0 Hz, 1H), 4.70 (s, 4H), 3.89 (s, 2H), 3.82 (s, 2H), 3.32 (s, 4H), 2.97-2.92 (m, 1H), 2.83 (s, 3H), 2.23-2.19 (m, 2H), 2.04-1.93 (m, 5H).

### Example 2

### 2-((6-(1,1-Difluoroethyl)-2-methylpyridin-3-yl)sulfonyl)-N-(tetrahydro-2H-pyran-4-yl)-2-azaspiro[3.3]heptan-6-amine

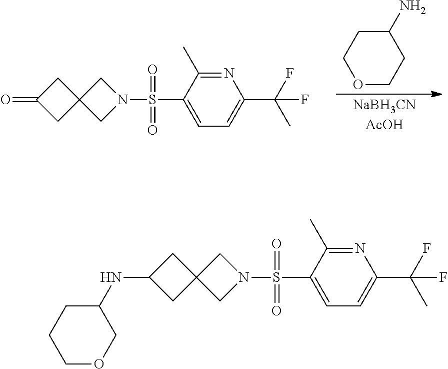

A solution of 2-((6-(1,1-difluoroethyl)-2-methylpyridin-3-yl)sulfonyl)-2-azaspiro[3.3]heptan-6-one (Example 1, step 5, 80 mg, 0.2 mmol) and tetrahydropyran-4-amine (37 mg, 0.4 mmol) in MeOH (3 mL) was adjusted to pH 5-6 using acetic acid and stirred for 0.5 h at 20 °C. NaBH₃CN (76 mg, 1.2 mmol) was added and the mixture was stirred for 1 h at 20 °C. The mixture was purified by prep-HPLC (Welch Xtimate C18 150×25 mm×5 μm, Condition: water (NH₄HCO₃)-MeCN, 37% to 52%, Flow Rate (mL/min): 25) to give 2-((6-(1,1-difluoroethyl)-2-methylpyridin-3-yl)sulfonyl)-N-(tetrahydro-2H-pyran-4-yl)-2-azaspiro[3.3]heptan-6-amine (25 mg, 25%) as white solid. LCMS m/z=416.2 [M+H]⁺. ¹H NMR (400 MHz, MeOH-d₄): δ ppm 8.36 (d, J=8.0 Hz, 1H), 7.71 (d, J=8.0 Hz, 1H), 3.93 (s, 2H), 3.90-3.88 (m, 2H), 3.81 (s, 2H), 3.40-3.34 (m, 2H), 3.28-3.25 (m, 1H), 2.84 (s, 3H), 2.70-2.65 (m, 1H), 2.45-2.43 (m, 2H), 2.05-1.94 (m, 5H), 1.77-1.72 (m, 2H), 1.37-1.35 (m, 2H).

### Example 3

### 2-((2-Methyl-6-(trifluoromethyl)pyridin-3-yl)sulfonyl)-N-(tetrahydro-2H-pyran-4-yl)-2-azaspiro[3.3]heptan-6-amine

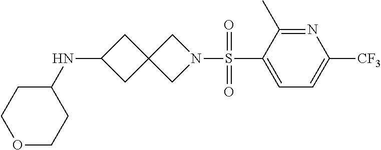 [SMILES: Cc1nc(C(F)(F)F)ccc1S(=O)(=O)N1CC2(CC(NC3CCOCC3)C2)C1]

### 1. Synthesis of 2-((2-methyl-6-(trifluoromethyl)pyridin-3-yl)sulfonyl)-2-azaspiro[3.3]heptan-6-one

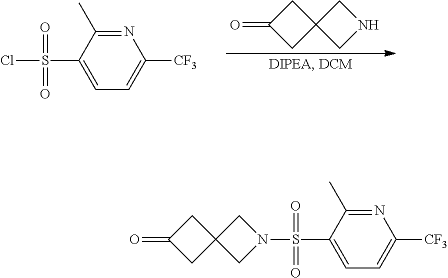

To a vial containing 2-azaspiro[3.3]heptan-6-one hydrochloride (300 mg, 2.0 mmol) in anhydrous DCM (8 mL) was added DIPEA (1.1 mL, 6.3 mmol) dropwise at <5 °C. After 5 mins, 2-methyl-6-(trifluoromethyl)pyridine-3-sulfonyl chloride (704 mg, 2.7 mmol) was added and the reaction was allowed to warm to 23 °C. After 30 mins, the reaction was quenched by slow addition of aqueous 1 M NaOH solution, the mixture was stirred at 23 °C for 20 mins, then the biphasic mixture was extracted with DCM (3×). The combined organic extracts were dried over anhydrous MgSO₄, filtered and concentrated under reduced pressure. The residue was purified by silica gel column chromatography (10-60% EtOAc in heptane) to give 2-((2-methyl-6-(trifluoromethyl)pyridin-3-yl)sulfonyl)-2-azaspiro[3.3]heptan-6-one (450 mg, 67%) as a white solid. LCMS m/z=335.0 [M+H]⁺.

### 2. Synthesis of 2-((2-methyl-6-(trifluoromethyl)pyridin-3-yl)sulfonyl)-N-(tetrahydro-2H-pyran-4-yl)-2-azaspiro[3.3]heptan-6-amine

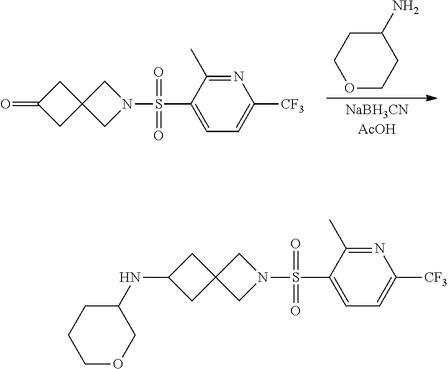

To a mixture of 2-((2-methyl-6-(trifluoromethyl)pyridin-3-yl)sulfonyl)-2-azaspiro[3.3]heptan-6-one (67 mg, 0.2 mmol) and tetrahydropyran-4-amine HCl salt (46 mg, 0.3 mmol) in DCM (3 mL) was added AcOH (34 μL, 0.6 mmol). NaBH(OAc)₃ (170 mg, 0.8 mmol) was added in one portion and the reaction mixture was stirred at rt for 3 days. The reaction was quenched with satd. NaHCO₃, stirred at rt for 5 min, more DCM and water added, and the mixture stirred for a further 5 min. The aqueous layer was removed, the organic phase was washed with water and concentrated. The residue was purified by silica gel column (50 to 100% EtOAc/EtOH (3/1)) to give 2-((2-methyl-6-(trifluoromethyl)pyridin-3-yl)sulfonyl)-N-(tetrahydro-2H-pyran-4-yl)-2-azaspiro[3.3]heptan-6-amine (61 mg, 73%) as a white solid. LCMS m/z=420.2 [M+H]⁺. 1H NMR (400 MHz, MeOH-d₄): δ (ppm) 8.47 (d, J=8.0 Hz, 1H), 7.85 (d, J=8.0 Hz, 1H), 4.01 (s, 2H), 3.95 (dd, J=11.0, 4.0 Hz, 2H), 3.89 (s, 2H), 3.50-3.36 (m, 3H), 2.89 (s, 3H), 2.85 (s, 1H), 2.58-2.48 (m, 2H), 2.16-2.06 (m, 2H), 1.88-1.77 (m, 2H), 1.44 (qd, J=12.2, 4.5 Hz, 2H)

### Example 4

### 6-(2-((2-Methyl-6-(trifluoromethyl)pyridin-3-yl)sulfonyl)-2-azaspiro[3.3]heptan-6-yl)-2-oxa-6-azaspiro[3.3]heptane

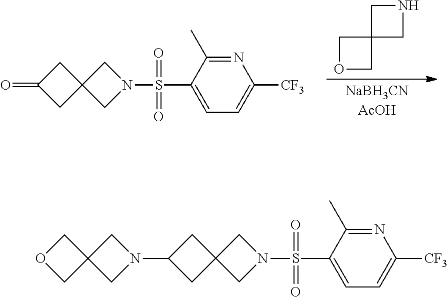

2-((2-Methyl-6-(trifluoromethyl)pyridin-3-yl)sulfonyl)-2-azaspiro[3.3]heptan-6-one (Example 3, step 1, 75 mg, 0.2 mmol) and 2-oxa-6-azaspiro[3.3]heptane (22 mg, 0.2 mmol) were dissolved in MeOH (6 mL). NaBH₃CN (71 mg, 1.1 mmol) and AcOH (30 μL, 0.5 mmol) were added and the reaction stirred at rt for 6 h. The reaction was quenched with ammonium chloride (sat. aq.), the mixture diluted with DCM and the layers separated. The organic phase was washed with water and brine and the solvent removed in vacuo. The crude product was purified by silica gel column (0-100% EtOAc to 3:1 EtOAc:EtOH (w/2% NH₃OH)) to give 6-(2-((2-methyl-6-(trifluoromethyl)pyridin-3-yl)sulfonyl)-2-azaspiro[3.3]heptan-6-yl)-2-oxa-6-azaspiro[3.3]heptane (65 mg, 69%). LCMS m/z=418.3 [M+H]⁺. ¹H NMR (500 MHz, CDCl₃): δ ppm 8.36-8.13 (m, 1H), 7.58 (d, J=8.2 Hz, 1H), 4.64 (s, 4H), 3.83 (d, J=16.5 Hz, 4H), 3.20 (s, 4H), 2.86-2.70 (m, 4H), 2.22-2.05 (m, 2H), 1.86 (m, 2H).

### Example 5

### 4-(2-((2-Methyl-6-(trifluoromethyl)pyridin-3-yl)sulfonyl)-2-azaspiro[3.3]heptan-6-yl)morpholine

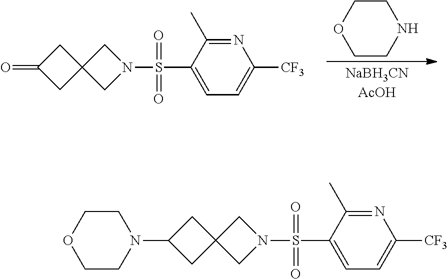

4-(2-((2-Methyl-6-(trifluoromethyl)pyridin-3-yl)sulfonyl)-2-azaspiro[3.3]heptan-6-yl)morpholine was obtained (9 mg, 10%), from 2-((2-methyl-6-(trifluoromethyl)pyridin-3-yl)sulfonyl)-2-azaspiro[3.3]heptan-6-one (Example 3, step 1) and morpholine, following the procedure described in Example 4. LCMS m/z=406.3 [M+H]⁺. ¹H NMR (500 MHz, CDCl₃): δ ppm 8.29 (d, J=7.9 Hz, 1H), 7.58 (d, J=7.9 Hz, 1H), 3.92 (s, 2H), 3.81 (s, 2H), 3.63 (br s, 4H), 2.83 (s, 3H), 2.55 (br t, J=7.6 Hz, 1H), 2.32-2.15 (m, 6H), 1.99 (br t, J=9.5 Hz, 2H).

### Example 6

### 6-(2-((2-Methyl-6-(trifluoromethyl)pyridin-3-yl)sulfonyl)-2-azaspiro[3.3]heptan-6-yl)-1-oxa-6-azaspiro[3.3]heptane

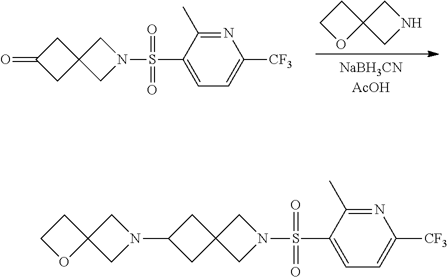

6-(2-((2-Methyl-6-(trifluoromethyl)pyridin-3-yl)sulfonyl)-2-azaspiro[3.3]heptan-6-yl)-1-oxa-6-azaspiro[3.3]heptane was obtained (67 mg, 72%), from 2-((2-methyl-6-(trifluoromethyl)pyridin-3-yl)sulfonyl)-2-azaspiro[3.3]heptan-6-one (Example 3, step 1) and 1-oxa-6-azaspiro[3.3]heptane, following the procedure described in Example 4. LCMS m/z=418.3 [M+H]⁺. ¹H NMR (500 MHz, CDCl₃): δ ppm 8.28 (d, J=8.2 Hz, 1H), 7.57 (d, J=7.9 Hz, 1H), 4.43 (t, J=7.6 Hz, 2H), 3.92-3.74 (m, 4H), 3.45 (br d, J=8.6 Hz, 2H), 3.02 (br d, J=9.2 Hz, 2H), 2.92-2.85 (m, 1H), 2.82 (s, 3H), 2.76 (t, J=7.5 Hz, 2H), 2.26-2.10 (m, 2H), 1.92-1.81 (m, 2H).

### Example 7

### 2-((2-Methyl-6-(trifluoromethyl)pyridin-3-yl)sulfonyl)-N-(1-(tetrahydro-2H-pyran-4-yl)cyclopropyl)-2-azaspiro[3.3]heptan-6-amine

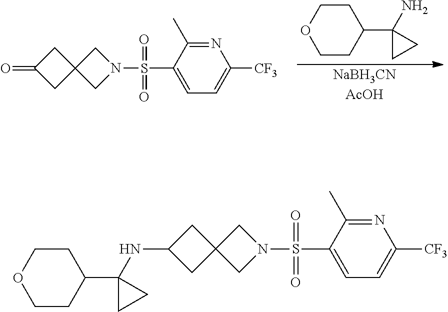

2-((2-Methyl-6-(trifluoromethyl)pyridin-3-yl)sulfonyl)-N-(1-(tetrahydro-2H-pyran-4-yl)cyclopropyl)-2-azaspiro[3.3]heptan-6-amine was obtained (80 mg, 39%), from 2-((2-methyl-6-(trifluoromethyl)pyridin-3-yl)sulfonyl)-2-azaspiro[3.3]heptan-6-one (Example 3, step 1) and 1-(tetrahydro-2H-pyran-4-yl)cyclopropan-1-amine following the procedure described in Example 4. LCMS m/z=460.3 [M+H]⁺. ¹H NMR (400 MHz, MeOH-d₄): δ ppm 8.38 (d, J=8.5 Hz, 1H), 7.77 (d, J=8.0 Hz, 1H), 4.70 (br s, 1H), 4.00-3.70 (m, 5H), 3.36-3.14 (m, 4H), 2.80 (s, 3H), 2.46-2.31 (m, 2H), 1.97-1.85 (m, 2H), 1.72-1.55 (m, 1H), 1.54-1.43 (m, 2H), 1.27-1.03 (m, 2H), 0.45-0.33 (m, 4H).

### Example 8

### 2-((2-Methyl-6-(trifluoromethyl)pyridin-3-yl)sulfonyl)-N-(oxetan-3-yl)-2-azaspiro[3.3]heptan-6-amine

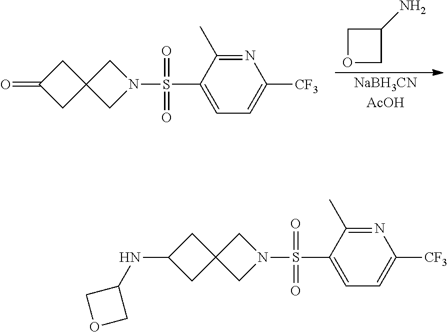

2-((2-Methyl-6-(trifluoromethyl)pyridin-3-yl)sulfonyl)-2-azaspiro[3.3]heptan-6-one (Example 3, step 1) (80 mg, 0.2 mmol) was dissolved in MeOH (1 mL) and DCM (3 mL). AcOH (30 μL, 0.5 mmol) was added, followed 15 mins later by NaBH₃CN (75 mg, 1.2 mmol) and the reaction stirred at rt for 24 h. The reaction was diluted with EtOAc and quenched with NaHCO₃ (sat. aq.) and the mixture extracted with EtOAc (3×). The combined organic extracts were washed with water, then concentrated in vacuo. The residue was purified by silica gel column chromatography with 0-100% EtOAc to 3:1 EtOAc:EtOH (w/2% NH₃OH) to give 2-((2-methyl-6-(trifluoromethyl)pyridin-3-yl)sulfonyl)-N-(oxetan-3-yl)-2-azaspiro[3.3]heptan-6-amine (63 mg, 67%). LCMS m/z=392.0 [M+H]⁺. ¹H NMR (500 MHz, CDCl₃): δ ppm 8.28 (d, J=8.2 Hz, 1H), 7.58 (d, J=8.2 Hz, 1H), 4.71 (t, J=6.7 Hz, 2H), 4.32 (t, J=6.4 Hz, 2H), 3.94-3.77 (m, 5H), 3.10 (t, J=7.3 Hz, 1H), 2.83 (s, 3H), 2.36 (m, 2H), 1.84 (m, 3H).

### Example 9

### 2-((2-Methyl-6-(trifluoromethyl)pyridin-3-yl)sulfonyl)-N-((3-methyloxetan-3-yl)methyl)-2-azaspiro[3.3]heptan-6-amine

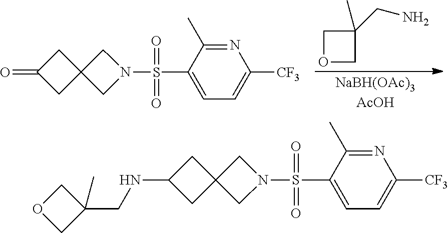

2-((2-Methyl-6-(trifluoromethyl)pyridin-3-yl)sulfonyl)-2-azaspiro[3.3]heptan-6-one (Example 3, step 1) (69 mg, 0.2 mmol) was dissolved in MeOH (1 mL) and DCM (3 mL). (3-Methyloxetan-3-yl)methanamine (25 mg, 0.3 mmol) and AcOH (25 μL, 0.4 mmol) were added and the solution stirred for 15 mins. NaBH(OAc)₃ (220 mg, 1.0 mmol) was added and the reaction stirred at rt for 24 h. The reaction was diluted with EtOAc and quenched with aq. NaHCO₃ solution. The solution was extracted with EtOAc (3×), the combined organic extracts were washed with water and concentrated in vacuo. The crude product was purified by column chromatography on silica gel eluting with (0-100%: EtOAc:EtOH (3:1) with 0.2% NH₃OH) in EtOH to give 2-((2-methyl-6-(trifluoromethyl)pyridin-3-yl)sulfonyl)-N-((3-methyloxetan-3-yl)methyl)-2-azaspiro[3.3]heptan-6-amine (60 mg, 69%). LCMS m/z=420.1 [M+H]⁺. ¹H NMR (500 MHz, CDCl₃): δ ppm 8.36 (br d, J=7.9 Hz, 1H), 7.65 (br d, J=7.9 Hz, 1H), 4.51-4.28 (m, 4H), 4.04-3.78 (m, 4H), 3.19 (m, 1H), 2.91 (s, 3H), 2.67 (s, 2H), 2.46 (m, 2H), 2.03-1.77 (m, 2H), 1.27 (s, 4H).

### Example 10

### 4-(2-((1-Methyl-3-(trifluoromethyl)-1H-pyrazol-5-yl)sulfonyl)-2-azaspiro[3.3]heptan-6-yl)morpholine

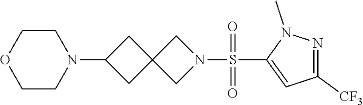 [SMILES: Cn1nc(C(F)(F)F)cc1S(=O)(=O)N1CC2(CC(N3CCOCC3)C2)C1]

### 1. Synthesis of 2-((1-methyl-3-(trifluoromethyl)-1H-pyrazol-5-yl)sulfonyl)-2-azaspiro[3.3]heptan-6-one

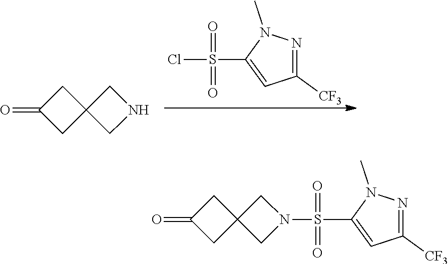 [SMILES: O=C1CC2(CNC2)C1 ; Cn1nc(C(F)(F)F)cc1S(=O)(=O)Cl ; Cn1nc(C(F)(F)F)cc1S(=O)(=O)N1CC2(CC(=O)C2)C1]

2-((1-Methyl-3-(trifluoromethyl)-1H-pyrazol-5-yl)sulfonyl)-2-azaspiro[3.3]heptan-6-one was obtained as a colorless film (404 mg, 62%), from 2-azaspiro[3.3]heptan-6-one and 2-methyl-5-(trifluoromethyl)pyrazole-3-sulfonyl chloride, following the procedure described in Example 3, step 1. ¹H NMR (400 MHz, DCM-d₂): δ ppm 7.05 (s, 1H), 4.16 (s, 3H), 4.12 (s, 4H), 3.28 (s, 4H).

### 2. Synthesis of 4-(2-((1-methyl-3-(trifluoromethyl)-1H-pyrazol-5-yl)sulfonyl)-2-azaspiro[3.3]heptan-6-yl)morpholine

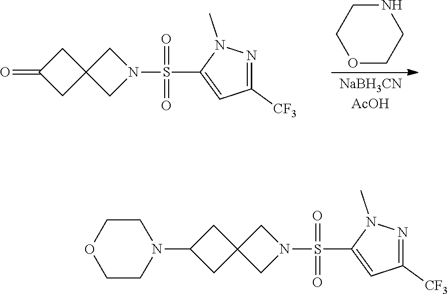 [SMILES: Cn1nc(C(F)(F)F)cc1S(=O)(=O)N1CC2(CC(=O)C2)C1 ; C1COCCN1 ; Cn1nc(C(F)(F)F)cc1S(=O)(=O)N1CC2(CC(N3CCOCC3)C2)C1]

2-((1-Methyl-3-(trifluoromethyl)-1H-pyrazol-5-yl)sulfonyl)-2-azaspiro[3.3]heptan-6-one (75 mg, 0.2 mmol) and morpholine hydrochloride (32 mg, 0.3 mmol) were dissolved in DCM (4 mL). NaBH₃CN (73 mg, 1.2 mmol) and AcOH (30 μL, 0.5 mmol) were added and the reaction stirred at rt for 6 h. The reaction was quenched with ammonium chloride (sat. aq.) and diluted with DCM. The layers were separated, the organic phase was washed with water and brine and then concentrated in vacuo. The residue was purified by silica gel column (0-100% EtOAc to 3:1 EtOAc:EtOH (w/2% NH₃OH)) to give 4-(2-((1-methyl-3-(trifluoromethyl)-1H-pyrazol-5-yl)sulfonyl)-2-azaspiro[3.3]heptan-6-yl)morpholine (25 mg, 27%). LCMS m/z=395.3 [M+H]⁺. ¹H NMR (500 MHz, CDCl₃): δ ppm 6.92 (s, 1H), 4.07 (s, 3H), 3.87 (s, 2H), 3.78 (s, 2H), 3.62 (br s, 4H), 2.54 (br t, J=7.6 Hz, 1H), 2.32-2.16 (m, 6H), 1.98 (br d, J=7.9 Hz, 2H).

### Example 11

### 2-((1-Methyl-3-(trifluoromethyl)-1H-pyrazol-5-yl)sulfonyl)-N-(oxetan-3-ylmethyl)-2-azaspiro[3.3]heptan-6-amine

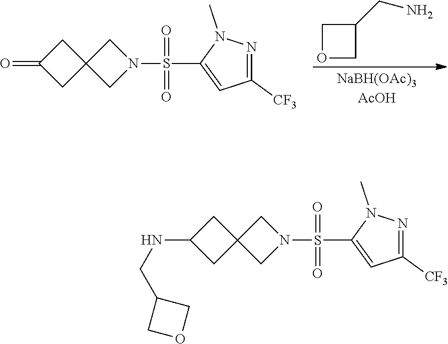 [SMILES: Cn1nc(C(F)(F)F)cc1S(=O)(=O)N1CC2(CC(=O)C2)C1 ; NCC1COC1 ; Cn1nc(C(F)(F)F)cc1S(=O)(=O)N1CC2(CC(NCC3COC3)C2)C1]

2-((1-Methyl-3-(trifluoromethyl)-1H-pyrazol-5-yl)sulfonyl)-N-(oxetan-3-ylmethyl)-2-azaspiro[3.3]heptan-6-amine was obtained (67 mg, 68%), from 2-((1-methyl-3-(trifluoromethyl)-1H-pyrazol-5-yl)sulfonyl)-2-azaspiro[3.3]heptan-6-one (Example 10, step 1) and oxetan-3-ylmethanamine, following the procedure described in Example 8. LCMS m/z=395.1 [M+H]⁺. ¹H NMR (500 MHz, CDCl₃): δ ppm 6.99 (s, 1H), 5.55 (br s, 1H), 4.79 (br t, J=6.9 Hz, 2H), 4.36 (br t, J=6.0 Hz, 2H), 4.15 (s, 3H), 3.92 (s, 2H), 3.86 (s, 2H), 3.22-3.11 (m, 1H), 3.06-2.95 (m, 1H), 2.82 (br d, J=7.3 Hz, 2H), 2.43 (m, 2H), 1.90-1.81 (m, 2H).

### Example 12

### 2-((1-Methyl-3-(trifluoromethyl)-1H-pyrazol-5-yl)sulfonyl)-N-((3-methyloxetan-3-yl)methyl)-2-azaspiro[3.3]heptan-6-amine

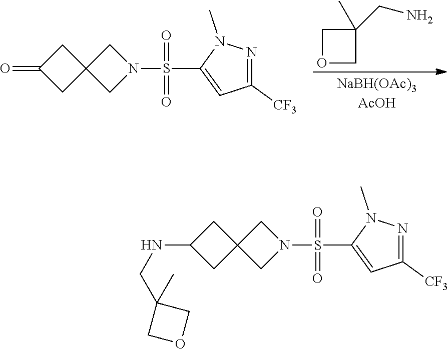

2-((1-Methyl-3-(trifluoromethyl)-1H-pyrazol-5-yl)sulfonyl)-N-((3-methyloxetan-3-yl)methyl)-2-azaspiro[3.3]heptan-6-amine was obtained (60 mg, 59%), from 2-((1-methyl-3-(trifluoromethyl)-1H-pyrazol-5-yl)sulfonyl)-2-azaspiro[3.3]heptan-6-one (Example 10, step 1) and (3-methyloxetan-3-yl)methanamine, following the procedure described in Example 8.

LCMS m/z=409.1 [M+H]⁺. ¹H NMR (500 MHz, CDCl₃): δ ppm 7.00 (s, 1H), 4.43-4.33 (m, 4H), 4.15 (s, 3H), 3.92 (s, 2H), 3.90-3.85 (m, 2H), 3.18 (m, 1H), 2.66 (s, 2H), 2.46-2.38 (m, 2H), 1.91-1.83 (m, 2H), 1.27 (s, 4H).

### Example 13

### 2-((1-Methyl-3-(trifluoromethyl)-1H-pyrazol-5-yl)sulfonyl)-N-(oxetan-3-yl)-2-azaspiro[3.3]heptan-6-amine

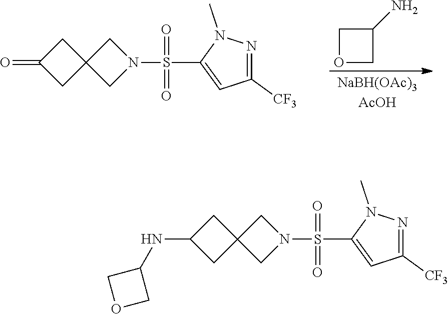 [SMILES: Cn1nc(C(F)(F)F)cc1S(=O)(=O)N1CC2(CC(=O)C2)C1 ; NC1COC1 ; Cn1nc(C(F)(F)F)cc1S(=O)(=O)N1CC2(CC(NC3COC3)C2)C1]

2-((1-Methyl-3-(trifluoromethyl)-1H-pyrazol-5-yl)sulfonyl)-2-azaspiro[3.3]heptan-6-one (Example 10, step 1, 80 mg, 0.3 mmol) was dissolved in MeOH (1 mL) and DCM (3 mL). Oxetan-3-amine (22 mg, 0.3 mmol) and AcOH (30 μL, 0.5 mmol) were added, the solution stirred for 15 mins, then NaBH₃CN (78 mg, 1.2 mmol) added. The reaction was stirred at rt for 24 h, then diluted with EtOAc and quenched with aq. NaHCO₃ solution. The solution was extracted with EtOAc (3×), the combined organic extracts washed with water and concentrated in vacuo. The crude product was purified by column chromatography eluting with (0-100%: EtOAc:EtOH (3:1) with 0.2% NH₃OH) in EtOH to give 2-((1-methyl-3-(trifluoromethyl)-1H-pyrazol-5-yl)sulfonyl)-N-(oxetan-3-yl)-2-azaspiro[3.3]heptan-6-amine (52 mg, 55%). LCMS m/z=381.0 [M+H]⁺. ¹H NMR (500 MHz, CDCl₃): δ ppm 6.92 (s, 1H), 5.59-5.35 (m, 1H), 4.71 (t, J=6.9 Hz, 2H), 4.31 (t, J=6.4 Hz, 2H), 4.07 (s, 3H), 3.89-3.73 (m, 5H), 3.14-3.03 (m, 1H), 2.32 (m, 2H), 1.81 (m, 2H).

### Example 14

### 6-(2-((1-Methyl-3-(trifluoromethyl)-1H-pyrazol-5-yl)sulfonyl)-2-azaspiro[3.3]heptan-6-yl)-2-oxa-6-azaspiro[3.3]heptane

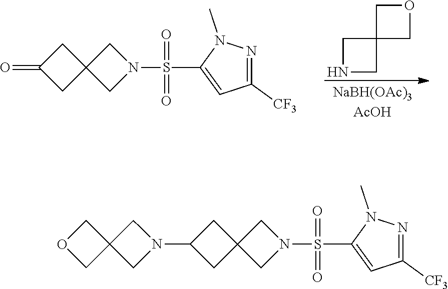 [SMILES: Cn1nc(C(F)(F)F)cc1S(=O)(=O)N1CC2(CC(=O)C2)C1 ; C1NCC12COC2 ; Cn1nc(C(F)(F)F)cc1S(=O)(=O)N1CC2(CC(N3CC4(COC4)C3)C2)C1]

6-(2-((1-Methyl-3-(trifluoromethyl)-1H-pyrazol-5-yl)sulfonyl)-2-azaspiro[3.3]heptan-6-yl)-2-oxa-6-azaspiro[3.3]heptane was obtained (8 mg, 8%), from 2-((1-methyl-3-(trifluoromethyl)-1H-pyrazol-5-yl)sulfonyl)-2-azaspiro[3.3]heptan-6-one (Example 10, step 1) and 2-oxa-6-azaspiro[3.3]heptane, following the procedure described in Example 4. LCMS m/z=407.3 [M+H]⁺. ¹H NMR (500 MHz, CDCl₃): δ ppm 6.91 (s, 1H), 4.64 (s, 4H), 4.07 (s, 3H), 3.79 (d, J=7.9 Hz, 4H), 3.18 (s, 4H), 2.76 (m, 1H), 2.17-2.01 (m, 2H), 1.86-1.72 (m, 2H).

### Example 15

### 6-(2-((1-Methyl-3-(trifluoromethyl)-1H-pyrazol-5-yl)sulfonyl)-2-azaspiro[3.3]heptan-6-yl)-1-oxa-6-azaspiro[3.3]heptane

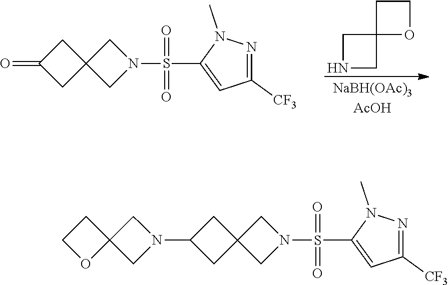 [SMILES: Cn1nc(C(F)(F)F)cc1S(=O)(=O)N1CC2(CC(=O)C2)C1 ; C1CC2(CNC2)O1 ; Cn1nc(C(F)(F)F)cc1S(=O)(=O)N1CC2(CC(N3CC4(CCO4)C3)C2)C1]

6-(2-((1-Methyl-3-(trifluoromethyl)-1H-pyrazol-5-yl)sulfonyl)-2-azaspiro[3.3]heptan-6-yl)-1-oxa-6-azaspiro[3.3]heptane was obtained (37 mg, 39%), from 2-((1-methyl-3-(trifluoromethyl)-1H-pyrazol-5-yl)sulfonyl)-2-azaspiro[3.3]heptan-6-one (Example 10, step 1) and 1-oxa-6-azaspiro[3.3]heptane following the procedure described in Example 4. LCMS m/z=407.3 [M+H]⁺. ¹H NMR (500 MHz, CDCl₃): δ ppm 6.91 (s, 1H), 4.43 (t, J=7.5 Hz, 2H), 4.14-3.95 (m, 3H), 3.79 (d, J=13.1 Hz, 4H), 3.49-3.32 (m, 2H), 2.99 (br d, J=9.5 Hz, 2H), 2.90-2.79 (m, 1H), 2.76 (t, J=7.5 Hz, 2H), 2.26-2.06 (m, 2H), 1.88-1.75 (m, 2H).

### Example 16

### 6-(2-((4-Methyl-2-(trifluoromethyl)pyrimidin-5-yl)sulfonyl)-2-azaspiro[3.3]heptan-6-yl)-1-oxa-6-azaspiro[3.3]heptane

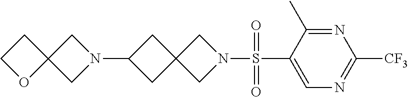 [SMILES: Cc1nc(C(F)(F)F)ncc1S(=O)(=O)N1CC2(CC(N3CC4(CCO4)C3)C2)C1]

### 1. Synthesis of 5-(benzylthio)-4-methyl-2-(trifluoromethyl)pyrimidine

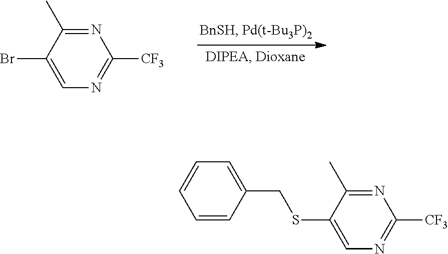 [SMILES: Cc1nc(C(F)(F)F)ncc1Br ; Cc1nc(C(F)(F)F)ncc1SCc1ccccc1]

To a solution of 5-bromo-4-methyl-2-(trifluoromethyl)pyrimidine (600 mg, 2.5 mmol) and BnSH (550 μL, 4.5 mmol) in dioxane (15 mL) was added DIPEA (1.3 mL, 7.5 mmol) and Pd(tBu₃P)₂ (190.8 mg, 0.373 mmol) and the reaction mixture was stirred at 100 °C for 12 h under N₂ atmosphere. The mixture was diluted with water (15 mL) and extracted with EtOAc (10 mL×3). The combined organic phase was washed with brine (30 mL), dried over anhydrous sodium sulfate, filtered and concentrated in vacuum. The crude was purified by column chromatography (EtOAc in PE=0 to 8%) to give 5-(benzylthio)-4-methyl-2-(trifluoromethyl)pyrimidine (670 mg, 95% yield) as yellow solid. LCMS m/z=285.0 [M+H]⁺

### 2. Synthesis of 4-methyl-2-(trifluoromethyl)pyrimidine-5-sulfonyl chloride

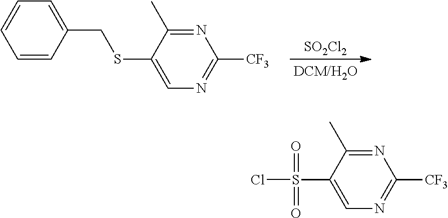 [SMILES: Cc1nc(C(F)(F)F)ncc1SCc1ccccc1 ; "SO2Cl2" ; Cc1nc(C(F)(F)F)ncc1S(=O)(=O)Cl]

To a solution of 5-(benzylthio)-4-methyl-2-(trifluoromethyl)pyrimidine (550 mg, 1.9 mmol) in DCM (5 mL) and water (1 mL) was added a solution of SO₂Cl₂ (1.10 mL, 13.6 mmol) in DCM (1 mL) dropwise over 5 mins at <5 °C and the reaction then stirred at <5 °C for 15 h. The mixture was diluted with water (10 mL) and extracted with EtOAc (15 mL×4). The combined organic phase was dried over anhydrous sodium sulfate, and concentrated in vacuo to give 4-methyl-2-(trifluoromethyl)pyrimidine-5-sulfonyl chloride (350 mg, crude) as a yellowish oil. ¹H NMR (400 MHz, CDCl₃): δ ppm 9.36 (s, 1H), 3.10 (s, 3H).

### 3. Synthesis of 2-((4-methyl-2-(trifluoromethyl)pyrimidin-5-yl)sulfonyl)-2-azaspiro[3.3]heptan-6-one

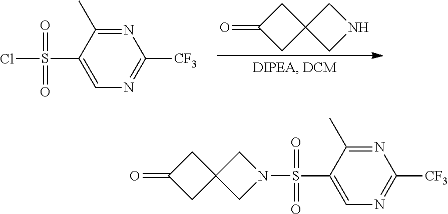 [SMILES: Cc1nc(C(F)(F)F)ncc1S(=O)(=O)Cl ; O=C1CC2(CNC2)C1 ; Cc1nc(C(F)(F)F)ncc1S(=O)(=O)N1CC2(CC(=O)C2)C1]

2-((4-Methyl-2-(trifluoromethyl)pyrimidin-5-yl)sulfonyl)-2-azaspiro[3.3]heptan-6-one was obtained (1.0 g, 34%), as a yellow solid, from 2-azaspiro[3.3]heptan-6-one (TFA salt) and 4-methyl-2-(trifluoromethyl)pyrimidine-5-sulfonyl chloride, following the procedure described in Example 1, step 4. LCMS m/z=336.0 [M+H]⁺. ¹H NMR (500 MHz, CDCl₃): δ ppm 9.21 (s, 1H), 4.25 (s, 4H), 3.36 (s, 4H), 2.93 (s, 3H).

### 4. Synthesis of 6-(2-((4-methyl-2-(trifluoromethyl)pyrimidin-5-yl)sulfonyl)-2-azaspiro[3.3]heptan-6-yl)-1-oxa-6-azaspiro[3.3]heptane

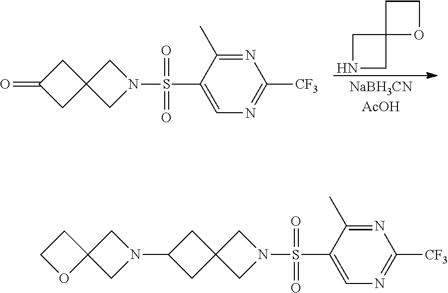

A solution of 2-((4-methyl-2-(trifluoromethyl)pyrimidin-5-yl)sulfonyl)-2-azaspiro[3.3]heptan-6-one (70 mg, 0.2 mmol) and 1-oxa-6-azaspiro[3.3]heptane hemioxalate (51 mg, 0.3 mmol) in MeOH (10 mL) was adjusted to pH 5-6 by the addition of AcOH and the reaction stirred for 0.5 h at 20 °C. NaBH₃CN (39 mg, 0.6 mmol) was added and the reaction was stirred for 1 h at 20 °C. The mixture was diluted with water (30 mL) and extracted with DCM (30 mL×3). The combined organic phase was dried over anhydrous sodium sulfate, filtered and concentrated in vacuo. The residue was purified by prep-HPLC (Phenomenex C18 150*40 mm*5 μm, Condition: water (NH₄HCO₃)-MeCN, 23% to 53%, Flow Rate (mL/min): 60) to give 6-(2-((4-methyl-2-(trifluoromethyl)pyrimidin-5-yl)sulfonyl)-2-azaspiro[3.3]heptan-6-yl)-1-oxa-6-azaspiro[3.3]heptane (25 mg, 29%) as white solid. LCMS m/z=419.1 [M+H]⁺. ¹H NMR (400 MHz, CDCl₃): δ ppm 9.17 (s, 1H), 4.50 (t, J=7.6 Hz, 2H), 3.98 (s, 2H), 3.95 (s, 2H), 3.51-3.49 (m, 2H), 3.08-3.06 (m, 2H), 2.96-2.93 (m, 1H), 2.90 (s, 3H), 2.83 (t, J=7.6 Hz, 2H), 2.26-2.21 (m, 2H), 1.98-1.93 (m, 2H).

### Example 17

### 6-(2-((4-Methyl-2-(trifluoromethyl)pyrimidin-5-yl)sulfonyl)-2-azaspiro[3.3]heptan-6-yl)-2-oxa-6-azaspiro[3.3]heptane

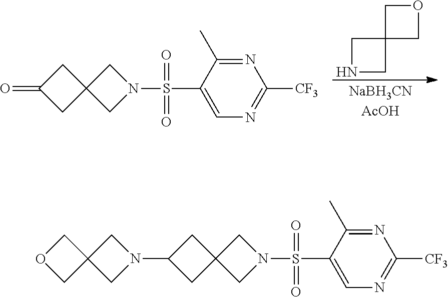

6-(2-((4-Methyl-2-(trifluoromethyl)pyrimidin-5-yl)sulfonyl)-2-azaspiro[3.3]heptan-6-yl)-2-oxa-6-azaspiro[3.3]heptane was obtained (27 mg, 31%), as a white solid, from 2-((4-methyl-2-(trifluoromethyl)pyrimidin-5-yl)sulfonyl)-2-azaspiro[3.3]heptan-6-one (Example 16, step 3) and 2-oxa-6-azaspiro[3.3]heptane oxalate following the procedure described in Example 1, step 4. LCMS m/z=419.1 [M+H]⁺. ¹H NMR (400 MHz, CDCl₃): δ ppm 9.17 (s, 1H), 4.71 (s, 4H), 3.99-3.95 (m, 4H), 3.26 (s, 4H), 2.90 (s, 3H), 2.87-2.82 (m, 1H), 2.23-2.18 (m, 2H), 1.95-1.90 (m, 2H).

### Example 18

### 2-Methyl-N-(2-(tetrahydro-2H-pyran-4-yl)-2-azaspiro[3.3]heptan-6-yl)-6-(trifluoromethyl)pyridine-3-sulfonamide

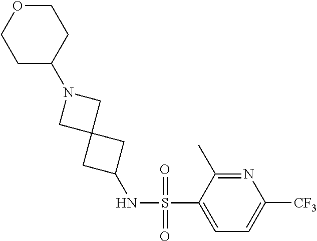 [SMILES: Cc1nc(C(F)(F)F)ccc1S(=O)(=O)NC1CC2(C1)CN(C1CCOCC1)C2]

### 1. Synthesis of tert-butyl 6-((2-methyl-6-(trifluoromethyl)pyridine)-3-sulfonamido)-2-azaspiro[3.3]heptane-2-carboxylate

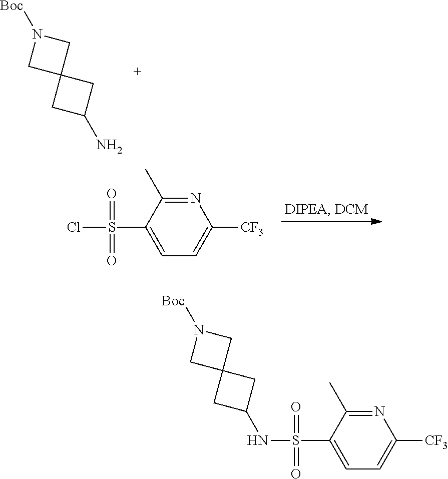 [SMILES: CC(C)(C)OC(=O)N1CC2(CC(N)C2)C1 ; Cc1nc(C(F)(F)F)ccc1S(=O)(=O)Cl ; Cc1nc(C(F)(F)F)ccc1S(=O)(=O)NC1CC2(C1)CN(C(=O)OC(C)(C)C)C2]

To a solution of tert-butyl 6-amino-2-azaspiro[3.3]heptane-2-carboxylate (1.24 g, 5.9 mmol) in anhydrous DCM (20 mL) was added DIPEA (4.0 mL, 23.0 mmol) dropwise at <5 °C. After 5 mins, 2-methyl-6-(trifluoromethyl)pyridine-3-sulfonyl chloride (2.01 g, 7.7 mmol) was added to the cold solution and upon complete addition, the reaction was allowed to warm to 23 °C and stirred for 90 mins. The reaction was quenched by slow addition of aqueous 1 M NaOH solution. The mixture was stirred at 23 °C for 20 mins, then the biphasic mixture was extracted with DCM (3×). The combined organic extracts were dried over anhydrous magnesium sulfate, the mixture filtered and concentrated under reduced pressure. The residue was loaded onto a silica gel column and purified with (10-60% EtOAc in heptane) to afford tert-butyl 6-((2-methyl-6-(trifluoromethyl)pyridine)-3-sulfonamido)-2-azaspiro[3.3]heptane-2-carboxylate (1.95 g, 77%) as a white solid. ¹H NMR (500 MHz, DMSO-d₆): δ ppm 8.56 (d, J=8.2 Hz, 1H), 8.38 (d, J=8.2 Hz, 1H), 7.96 (d, J=8.2 Hz, 1H), 3.82-3.65 (m, 4H), 3.58-3.51 (m, 1H), 2.82 (s, 3H), 2.26-2.20 (m, 2H), 2.01-1.95 (m, 2H), 1.32 (s, 9H).

### 2. Synthesis of 2-methyl-N-(2-azaspiro[3.3]heptan-6-yl)-6-(trifluoromethyl)pyridine-3-sulfonamide

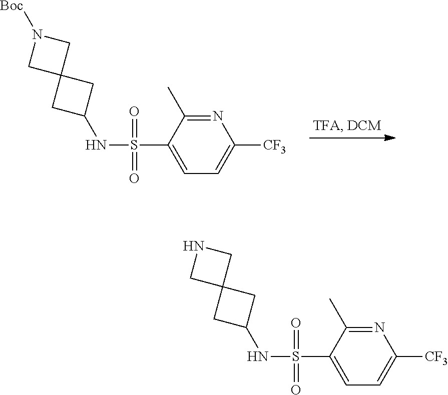 [SMILES: Cc1nc(C(F)(F)F)ccc1S(=O)(=O)NC1CC2(C1)CN(C(=O)OC(C)(C)C)C2 ; Cc1nc(C(F)(F)F)ccc1S(=O)(=O)NC1CC2(CNC2)C1]

To a solution of tert-butyl 6-((2-methyl-6-(trifluoromethyl)pyridine)-3-sulfonamido)-2-azaspiro[3.3]heptane-2-carboxylate (522 mg, 1.2 mmol) in anhydrous DCM (8 mL) was added TFA (0.6 mL, 7.8 mmol), dropwise at <5 °C. The reaction was stirred at rt for 21 h, then concentrated under reduced pressure to afford 2-methyl-N-(2-azaspiro[3.3]heptan-6-yl)-6-(trifluoromethyl)pyridine-3-sulfonamide as a light brown residue (606 mg, crude, TFA salt) that was used without purification. LCMS m/z=336.2 [M+H]⁺

### 3. Synthesis of 2-methyl-N-(2-(tetrahydro-2H-pyran-4-yl)-2-azaspiro[3.3]heptan-6-yl)-6-(trifluoromethyl)pyridine-3-sulfonamide

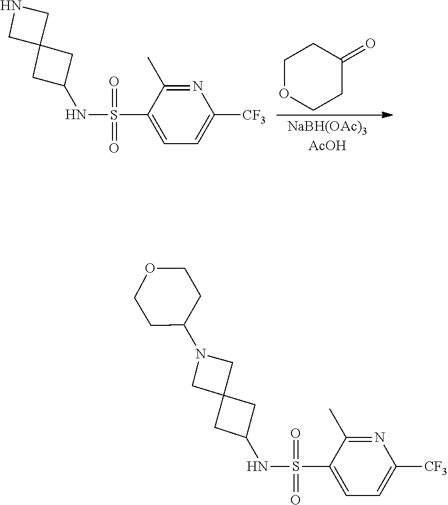

A solution of 2-methyl-N-(2-azaspiro[3.3]heptan-6-yl)-6-(trifluoromethyl)pyridine-3-sulfonamide (269 mg, 0.6 mmol, TFA salt) in anhydrous MeOH (4 mL) was cooled in an ice water bath, then DIPEA (0.7 mL, 4.0 mmol) was added and the solution stirred for 20 mins. Tetrahydropyran-4-one (120 μL, 1.30 mmol) and AcOH (300 μL, 4.9 mmol) were added to the cooled mixture, the solution stirred for 15 mins, then NaBH(OAc)₃ (803.3 mg, 3.79 mmol) was added in portions. The reaction was maintained at <5 °C and stirred for 2 h. The reaction was quenched by slow addition of aqueous saturated NaHCO₃ solution, the mixture was stirred at 23 °C for 30 mins, then extracted with DCM (3×). The combined organic extracts were dried over anhydrous MgSO₄, filtered and concentrated under reduced pressure. The residue was purified by silica gel column and purified with (40-100% 3:1 EtOAc:EtOH in heptane) to give 2-methyl-N-(2-(tetrahydro-2H-pyran-4-yl)-2-azaspiro[3.3]heptan-6-yl)-6-(trifluoromethyl)pyridine-3-sulfonamide (88 mg, 33.3% yield) as a colorless film. LCMS m/z=420.3 [M+H]⁺. ¹H NMR (500 MHz, DMSO-d₆): δ ppm 8.54 (br d, J=8.2 Hz, 1H), 8.38 (d, J=7.9 Hz, 1H), 7.97 (d, J=8.2 Hz, 1H), 3.75 (br dd, J=5.3, 1.1 Hz, 2H), 3.59-3.52 (m, 1H), 3.24-3.18 (m, 2H), 3.03-2.89 (m, 3H), 2.83 (s, 3H), 2.12 (br s, 2H), 2.05-1.87 (m, 4H), 1.59-1.41 (m, 2H), 1.07-1.00 (m, 2H).

### Example 19

### 4-(2-((3-Isopropyl-1-methyl-1H-pyrazol-5-yl)sulfonyl)-2-azaspiro[3.3]heptan-6-yl)morpholine

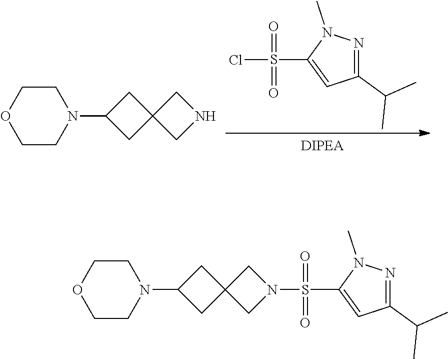 [SMILES: C1CN(C2CC3(CNC3)C2)CCO1 ; CC(C)c1cc(S(=O)(=O)Cl)n(C)n1 ; CC(C)c1cc(S(=O)(=O)N2CC3(CC(N4CCOCC4)C3)C2)n(C)n1]

To a mixture of 4-(2-azaspiro[3.3]heptan-6-yl)morpholine 2HCl salt (29 mg, 0.2 mmol) and 3-isopropyl-1-methyl-1H-pyrazole-5-sulfonyl chloride (36 mg, 0.2 mmol) in DCM (5 mL) was added DIPEA (110 μL, 0.6 mmol) and the reaction mixture was stirred at rt for 3 h. The reaction was quenched with satd. NaHCO₃, diluted with water, and stirred at rt for 5 min. The aqueous layer was removed and the organic phase was washed with water and concentrated. The residue was purified by silica gel column (50-100% EtOAc/EtOH 3/1 in heptane) to give 4-(2-((3-isopropyl-1-methyl-1H-pyrazol-5-yl)sulfonyl)-2-azaspiro[3.3]heptan-6-yl)morpholine (18 mg, 31%) as an off-white solid after lyophilization. LCMS m/z=369.2 [M+H]⁺. ¹H NMR (400 MHz, MeOH-d₄): δ (ppm) 6.68 (s, 1H), 4.04 (s, 3H), 3.89 (s, 2H), 3.78 (s, 2H), 3.72-3.63 (m, 4H), 3.08-2.90 (m, 1H), 2.73-2.57 (m, 1H), 2.32 (br s, 4H), 2.26-2.18 (m, 2H), 2.03-1.94 (m, 2H), 1.28 (d, J=7.0 Hz, 6H).

### Example 20

### 2-((4-(Difluoromethoxy)phenyl)sulfonyl)-6-(4-methylpiperidin-1-yl)-2-azaspiro[3.3]heptane

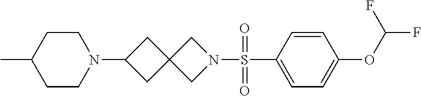 [SMILES: CC1CCN(C2CC3(C2)CN(S(=O)(=O)c2ccc(OC(F)F)cc2)C3)CC1]

### 1. Synthesis of tert-butyl 6-(4-methylpiperidin-1-yl)-2-azaspiro[3.3]heptane-2-carboxylate

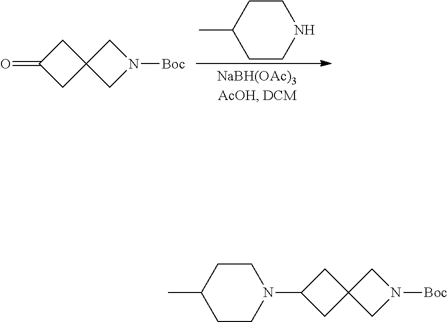

NaBH(OAc)₃ (307 mg, 1.5 mmol) was added to a mixture of 4-methylpiperidine (79 mg, 0.8 mmol), tert-butyl 6-oxo-2-azaspiro[3.3]heptane-2-carboxylate (153 mg, 0.7 mmol) and AcOH (50 μL, 0.8 mmol) in DCM (5 mL) and the reaction was stirred for 3 days at rt. The reaction mixture was washed with 10% aqueous solution of KOH and water, dried over Na₂SO₄ and the solvent was evaporated to give tert-butyl 6-(4-methylpiperidin-1-yl)-2-azaspiro[3.3]heptane-2-carboxylate, that was used in the next step without purification.

### 2. Synthesis of 6-(4-methylpiperidin-1-yl)-2-azaspiro[3.3]heptane

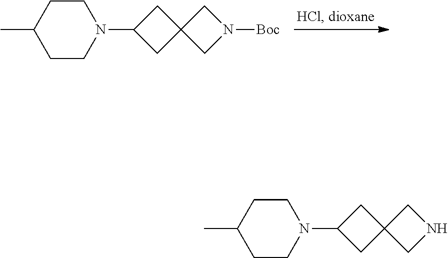 [SMILES: CC1CCN(C2CC3(C2)CN(C(=O)OC(C)(C)C)C3)CC1 ; CC1CCN(C2CC3(CNC3)C2)CC1]

tert-Butyl 6-(4-methylpiperidin-1-yl)-2-azaspiro[3.3]heptane-2-carboxylate was dissolved in DCM (2 mL) and 10% HCl in dioxane (1.5 mL) was added. The solution was placed in an ultrasonic bath (40 °C) for 4 h, then was stirred for 16 h at rt. The solvent was evaporated in vacuo to give 6-(4-methylpiperidin-1-yl)-2-azaspiro[3.3]heptane as HCl salt that was used in the next step without purification.

### 3. Synthesis of 2-((4-(difluoromethoxy)phenyl)sulfonyl)-6-(4-methylpiperidin-1-yl)-2-azaspiro[3.3]heptane

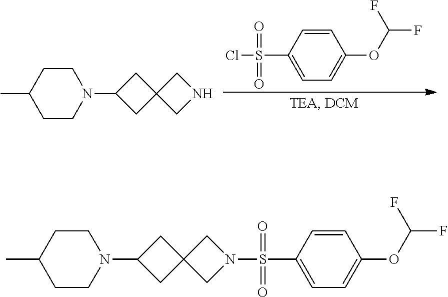

4-(Difluoromethoxy)benzene-1-sulfonyl chloride (176 mg, 0.7 mmol) was added to a mixture of 6-(4-methylpiperidin-1-yl)-2-azaspiro[3.3]heptane as hydrochloride salt and TEA (500 μL, 3.6 mmol) in dry DCM (5 mL) at −10 °C. The reaction was warmed to rt and stirred for 16 h at this temperature. The reaction mixture was washed with water, the solvent was removed in vacuo, the residue was dissolved in DMSO (1 mL) and purified by prep. HPLC (Waters SunFire C18 19*100 5 mkm column; H₂O-MeCN as a mobile phase) to afford 2-((4-(difluoromethoxy)phenyl)sulfonyl)-6-(4-methylpiperidin-1-yl)-2-azaspiro[3.3]heptane. LCMS m/z=401.2 [M+H]^{+ 1}H NMR (400 MHz, CDCl₃): δ ppm 8.23 (d, J=1.6 Hz, 1H), 7.99 (d, J=2.0 Hz, 1H), 4.03 (s, 3H), 4.03-3.96 (m, 2H), 3.95-3.89 (m, 5H), 3.58-3.50 (m, 2H), 3.43-3.36 (m, 2H), 2.70-2.65 (m, 1H), 2.40-2.34 (m, 1H), 2.33 (s, 3H), 1.98-1.92 (m, 1H), 1.85-1.79 (m, 2H), 1.38-1.30 (m, 2H).

### Example 21

### 2-((2-Methoxy-5-methylpyridin-3-yl)sulfonyl)-N-(tetrahydro-2H-pyran-4-yl)-5-oxa-2-azaspiro[3.4]octan-7-amine

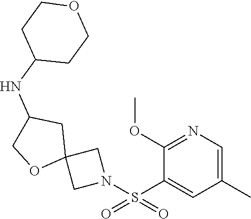 [SMILES: COc1ncc(C)cc1S(=O)(=O)N1CC2(CC(NC3CCOCC3)CO2)C1]

### 1. Synthesis of 5-oxa-2-azaspiro[3.4]octan-7-one

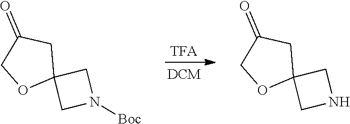 [SMILES: CC(C)(C)OC(=O)N1CC2(CC(=O)CO2)C1 ; O=C1COC2(CNC2)C1]

To a solution of tert-butyl 7-oxo-5-oxa-2-azaspiro[3.4]octane-2-carboxylate (900 mg, 4.0 mmol) in DCM (9 mL) was added TFA (3.0 mL, 39.2 mmol) and the reaction stirred at 25 °C for 3 h. The mixture was concentrated in vacuo to give 5-oxa-2-azaspiro[3.4]octan-7-one (900 mg, crude, TFA salt) as white oil which was used directly for the next step without further purification. ¹H NMR (400 MHz, MeOH-d₄): δ ppm 4.32-4.28 (m, 1H), 4.26-4.22 (m, 1H), 4.18-4.15 (m, 2H), 3.88 (d, J=8.8 Hz, 1H), 3.75 (d, J=8.8 Hz, 1H), 2.38 (s, 2H).

### 2. Synthesis of 2-((2-methoxy-5-methylpyridin-3-yl)sulfonyl)-5-oxa-2-azaspiro[3.4]octan-7-one

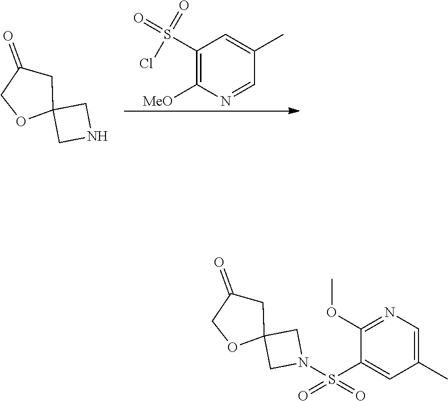 [SMILES: O=C1COC2(CNC2)C1 ; COc1ncc(C)cc1S(=O)(=O)Cl ; COc1ncc(C)cc1S(=O)(=O)N1CC2(CC(=O)CO2)C1]

To a solution of 5-oxa-2-azaspiro[3.4]octan-7-one (750 mg, 3.1 mmol, TFA salt) and DIPEA (1.6 mL, 9.4 mmol) in DCM (3 mL) was added 2-methoxy-5-methylpyridine-3-sulfonyl chloride (761.47 mg, 3.44 mmol) at 0 °C, then the mixture was stirred at 20 °C for 2 h under N₂. The mixture was diluted with water (10 mL) and extracted with DCM (15 mL×3). The combined organic phase was washed with brine (15 mL), dried over anhydrous sodium sulfate, and concentrated in vacuo. The crude was purified by silica gel chromatography (2-5% MeOH in DCM) to give 2-((2-methoxy-5-methylpyridin-3-yl)sulfonyl)-5-oxa-2-azaspiro[3.4]octan-7-one (650 mg, 67%) as brown oil. LCMS m/z=313.0 [M+H]⁺. ¹H NMR (500 MHz, MeOH-d₄): δ ppm 8.16-8.14 (m, 1H), 7.97 (s, 1H), 4.22 (d, J=9.5 Hz, 2H), 4.09 (d, J=9.5 Hz, 2H), 4.06 (s, 3H), 3.99 (s, 2H), 2.73 (s, 2H), 2.32 (s, 3H).

### 3. Synthesis of 2-((2-methoxy-5-methylpyridin-3-yl)sulfonyl)-N-(tetrahydro-2H-pyran-4-yl)-5-oxa-2-azaspiro[3.4]octan-7-amine

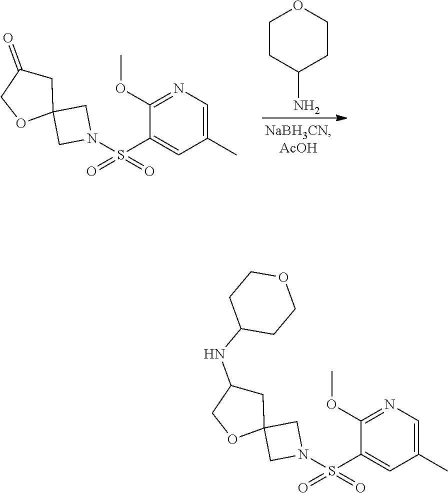 [SMILES: COc1ncc(C)cc1S(=O)(=O)N1CC2(CC(=O)CO2)C1 ; NC1CCOCC1 ; COc1ncc(C)cc1S(=O)(=O)N1CC2(CC(NC3CCOCC3)CO2)C1]

To a solution of 2-((2-methoxy-5-methylpyridin-3-yl)sulfonyl)-5-oxa-2-azaspiro[3.4]octan-7-one (120 mg, 0.4 mmol) and tetrahydro-2H-pyran-4-amine (58 mg, 0.6 mmol) in MeOH (6 mL) was added AcOH (25 μL, 0.4 mmol) and NaBH₃CN (121 mg, 1.9 mmol) at 20 °C, then the mixture was stirred at 20 °C for 15 h under N₂. The mixture was concentrated in vacuo and purified by prep-HPLC (Column: Boston Prime C18 150*30 mm*5 μm, Condition: water (NH₄HCO₃)-MeCN, 22% to 52%, Flow Rate (mL/min): 25) to give 2-((2-methoxy-5-methylpyridin-3-yl)sulfonyl)-N-(tetrahydro-2H-pyran-4-yl)-5-oxa-2-azaspiro[3.4]octan-7-amine (52 mg, 34%) as a white solid. LCMS m/z=398.1 [M+H]⁺. ¹H NMR (400 MHz, MeOH-d₄): δ ppm 8.23 (d, J=2.0 Hz, 1H), 7.99 (d, J=1.6 Hz, 1H), 4.03 (s, 3H), 4.03-3.96 (m, 2H), 3.95-3.89 (m, 5H), 3.58-3.50 (m, 2H), 3.43-3.36 (m, 2H), 2.70-2.65 (m, 1H), 2.40-2.34 (m, 1H), 2.33 (s, 3H), 1.98-1.92 (m, 1H), 1.85-1.79 (m, 2H), 1.38-1.30 (m, 2H).

### Example 22

### 2-((2-Methoxy-5-methylpyridin-3-yl)sulfonyl)-7-(2-oxa-6-azaspiro[3.3]heptan-6-yl)-5-oxa-2-azaspiro[3.4]octane

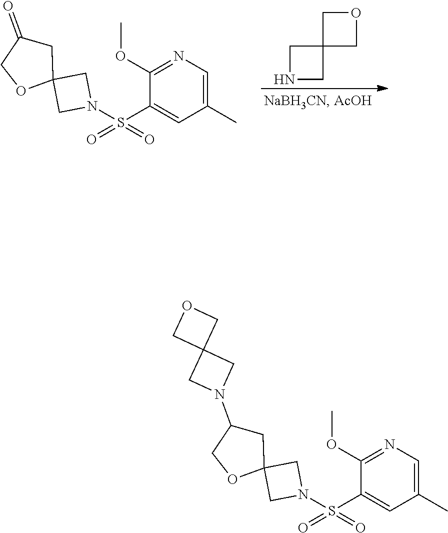 [SMILES: COc1ncc(C)cc1S(=O)(=O)N1CC2(CC(=O)CO2)C1 ; C1NCC12COC2 ; COc1ncc(C)cc1S(=O)(=O)N1CC2(CC(N3CC4(COC4)C3)CO2)C1]

2-((2-Methoxy-5-methylpyridin-3-yl)sulfonyl)-7-(2-oxa-6-azaspiro[3.3]heptan-6-yl)-5-oxa-2-azaspiro[3.4]octane was obtained as a brown oil (64 mg, 42%), from 2-((2-methoxy-5-methylpyridin-3-yl)sulfonyl)-5-oxa-2-azaspiro[3.4]octan-7-one (Example 21, step 3) and 2-oxa-6-azaspiro[3.3]heptane oxalate, following the procedure described in Example 21, step 4. LCMS m/z=396.1 [M+H]⁺. ¹H NMR (400 MHz, CDCl₃): δ ppm 8.23 (d, J=2.4 Hz, 1H), 7.99 (s, 1H), 4.70 (s, 4H), 4.03 (s, 3H), 4.01-3.98 (m, 1H), 3.95-3.89 (m, 3H), 3.68-3.66 (m, 1H), 3.58-3.55 (m, 1H), 3.38-3.35 (m, 2H), 3.32-3.30 (m, 2H), 2.99-2.95 (m, 1H), 2.33 (s, 3H), 2.09-2.04 (m, 1H), 1.98-1.93 (m, 1H).

### Example 23

### 2-((2-Methoxy-5-methylpyridin-3-yl)sulfonyl)-7-(3-methoxyazetidin-1-yl)-5-oxa-2-azaspiro[3.4]octane

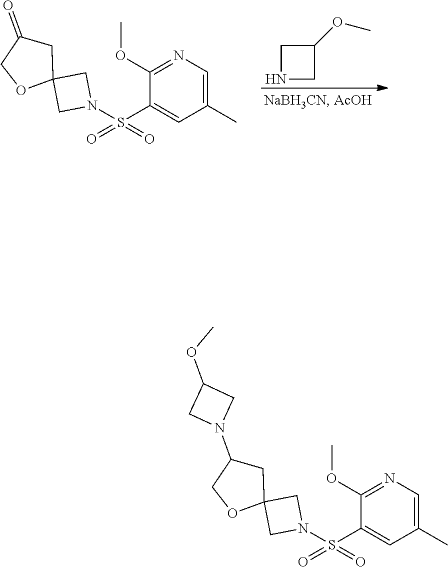

2-((2-Methoxy-5-methylpyridin-3-yl)sulfonyl)-7-(3-methoxyazetidin-1-yl)-5-oxa-2-azaspiro[3.4]octane was obtained as a brown oil (58 mg, 39%), from 2-((2-methoxy-5-methylpyridin-3-yl)sulfonyl)-5-oxa-2-azaspiro[3.4]octan-7-one (Example 21, step 3) and 3-methoxyazetidine hydrochloride, following the procedure described in Example 21, step 4. LCMS m/z=384.1 [M+H]⁺. ¹H NMR (400 MHz, CDCl₃): δ ppm 8.22 (s, 1H), 7.98 (d, J=2.0 Hz, 1H), 4.03 (s, 3H), 4.01-3.98 (m, 2H), 3.98-3.96 (m, 1H), 3.95-3.91 (m, 2H), 3.72-3.69 (m, 1H), 3.60-3.51 (m, 3H), 3.23 (s, 3H), 3.07-3.03 (m, 1H), 2.95-2.91 (m, 1H), 2.89-2.86 (m, 1H), 2.33 (s, 3H), 2.12-2.07 (m, 1H), 1.99-1.95 (m, 1H).

### Example 24

### N-(4,4-Difluorocyclohexyl)-2-((2-methoxy-5-methylpyridin-3-yl)sulfonyl)-5-oxa-2-azaspiro[3.4]octan-7-amine

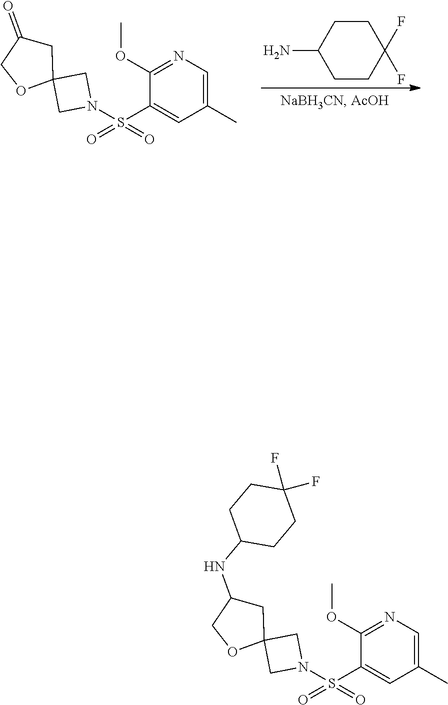 [SMILES: COc1ncc(C)cc1S(=O)(=O)N1CC2(CC(=O)CO2)C1 ; NC1CCC(F)(F)CC1 ; COc1ncc(C)cc1S(=O)(=O)N1CC2(CC(NC3CCC(F)(F)CC3)CO2)C1]

N-(4,4-Difluorocyclohexyl)-2-((2-methoxy-5-methylpyridin-3-yl)sulfonyl)-5-oxa-2-azaspiro[3.4]octan-7-amine was obtained as a white solid (42 mg, 19%), from 2-((2-methoxy-5-methylpyridin-3-yl)sulfonyl)-5-oxa-2-azaspiro[3.4]octan-7-one (Example 21, step 3) and 4,4-difluorocyclohexan-1-amine hydrochloride, following a similar procedure to that described in Example 21, step 4, except the crude product was purified by prep-HPLC (Column: Boston Prime C18 150×30 mm×5 μm, Condition: water (NH₄HCO₃)-MeCN, 32% to 62%, Flow Rate (mL/min): 25). LCMS m/z=432.1 [M+H]⁺. ¹H NMR (400 MHz, CDCl₃): δ ppm 8.13 (d, J=2.0 Hz, 1H), 7.95 (d, J=2.4 Hz, 1H), 4.07-4.04 (m, 1H), 4.03 (s, 3H), 4.03-4.00 (m, 2H), 3.96-3.88 (m, 2H), 3.54-3.46 (m, 2H), 2.60-2.56 (m, 1H), 2.29 (s, 3H), 2.28-2.23 (m, 1H), 2.10-1.99 (m, 3H), 1.90-1.71 (m, 4H), 1.45-1.36 (m, 3H).

### Example 25

### N-(3,3-Difluorocyclobutyl)-2-((2-methoxy-5-methylpyridin-3-yl)sulfonyl)-5-oxa-2-azaspiro[3.4]octan-7-amine

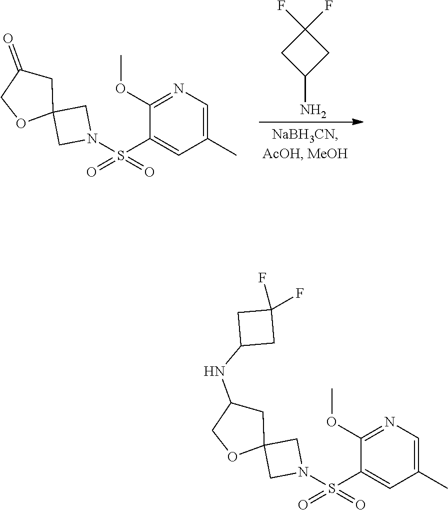 [SMILES: COc1ncc(C)cc1S(=O)(=O)N1CC2(CC(=O)CO2)C1 ; NC1CC(F)(F)C1 ; COc1ncc(C)cc1S(=O)(=O)N1CC2(CC(NC3CC(F)(F)C3)CO2)C1]

N-(3,3-Difluorocyclobutyl)-2-((2-methoxy-5-methylpyridin-3-yl)sulfonyl)-5-oxa-2-azaspiro[3.4]octan-7-amine was obtained as a white solid (78 mg, 31% yield), from 2-((2-methoxy-5-methylpyridin-3-yl)sulfonyl)-5-oxa-2-azaspiro[3.4]octan-7-one (Example 21, step 3) and 3,3-difluorocyclobutylamine, following a similar procedure to that described in Example 21, step 4, except the crude product was purified by prep-HPLC (Column: Boston Prime C18 150×30 mm×5 μm, Condition: water (NH₄HCO₃)-MeCN, 28% to 58%, Flow Rate (mL/min): 25). LCMS m/z=404.1 [M+H]^{+ 1}H NMR (400 MHz, CDCl₃): δ ppm 8.14-8.12 (m, 1H), 7.96 (d, J=2.0 Hz, 1H), 4.08-4.06 (m, 1H), 4.04 (s, 3H), 4.04-4.01 (m, 1H), 3.96 (d, J=8.4 Hz, 1H), 3.89-3.86 (m, 1H), 3.58-3.55 (m, 1H), 3.40-3.31 (m, 1H), 3.24-3.14 (m, 1H), 2.88-2.74 (m, 2H), 2.30 (s, 3H), 2.29-2.15 (m, 3H), 2.06-1.99 (m, 1H), 1.58 (br s, 2H).

### Example 26

### 3-((7-((4,4-Difluorocyclohexyl)amino)-5-oxa-2-azaspiro[3.4]octan-2-yl)sulfonyl)-4-fluorobenzonitrile

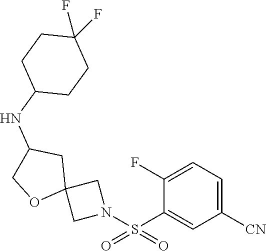 [SMILES: N#Cc1ccc(F)c(S(=O)(=O)N2CC3(CC(NC4CCC(F)(F)CC4)CO3)C2)c1]

### 1. Synthesis of 4-Fluoro-3-((7-oxo-5-oxa-2-azaspiro[3.4]octan-2-yl)sulfonyl)benzonitrile

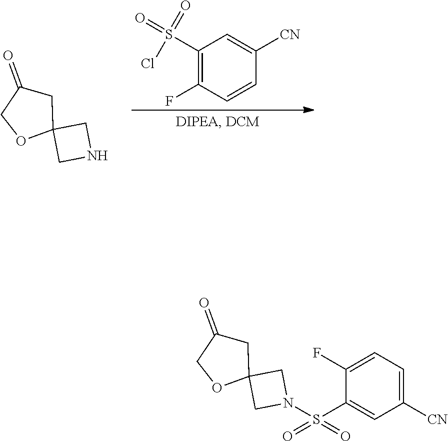 [SMILES: O=C1COC2(CNC2)C1 ; N#Cc1ccc(F)c(S(=O)(=O)Cl)c1 ; N#Cc1ccc(F)c(S(=O)(=O)N2CC3(CC(=O)CO3)C2)c1]

To a solution of 5-oxa-2-azaspiro[3.4]octan-7-one (Example 21, step 1, 900 mg, 3.8 mmol, TFA salt) in DCM (10 mL) was added DIPEA (2.0 mL, 11.2 mmol) and 5-cyano-2-fluorobenzenesulfonyl chloride (988 mg, 4.5 mmol) at 0 °C and the reaction stirred at 0 °C for 1 h. Water (50 mL) was added and the mixture extracted with DCM (30 mL×3). The combined organic layer was washed with brine (30 mL), dried over Na₂SO₄, filtered and concentrated. The crude product was purified by column chromatography (EtOAc in PE from 30% to 50%) to give 4-fluoro-3-((7-oxo-5-oxa-2-azaspiro[3.4]octan-2-yl)sulfonyl)benzonitrile (750 mg, 65%) as a white solid. LCMS m/z=311.0 [M+H]⁺

### 2. Synthesis of 3-((7-((4,4-difluorocyclohexyl)amino)-5-oxa-2-azaspiro[3.4]octan-2-yl)sulfonyl)-4-fluorobenzonitrile

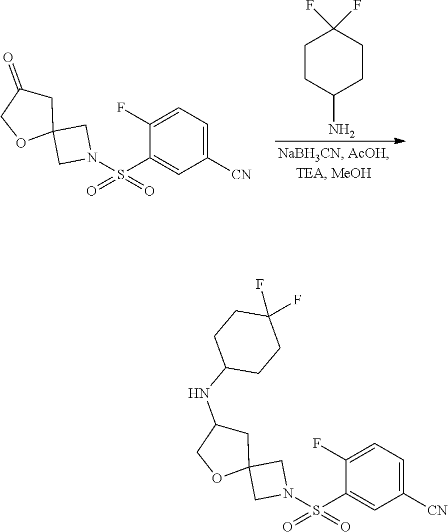 [SMILES: N#Cc1ccc(F)c(S(=O)(=O)N2CC3(CC(=O)CO3)C2)c1 ; NC1CCC(F)(F)CC1 ; N#Cc1ccc(F)c(S(=O)(=O)N2CC3(CC(NC4CCC(F)(F)CC4)CO3)C2)c1]

To a solution of 4-fluoro-3-((7-oxo-5-oxa-2-azaspiro[3.4]octan-2-yl)sulfonyl)benzonitrile (130 mg, 0.4 mmol) and 4,4-difluorocyclohexylamine (86 mg, 0.5 mmol, HCl salt) in MeOH (5 mL) was added TEA (120 μL, 0.8 mmol) and the solution was stirred at 20 °C for 20 mins. The pH of the solution was adjusted to 5-6 using AcOH and the solution stirred for 30 mins at 20 °C. NaBH₃CN (132 mg, 2.1 mmol) was added and the reaction stirred at 20 °C for 14 h. The mixture was diluted with water (50 mL) and extracted with DCM (50 mL×3). The combined organic phase was dried over anhydrous sodium sulfate, filtered and concentrated in vacuo. The crude product was purified by prep-HPLC ((Column: Boston Prime C18 150×30 mm×5 μm, Condition: water (NH₃·H₂O+NH₄HCO₃)-MeCN, 41% to 71%, Flow Rate (mL/min): 25)) to give 3-((7-((4,4-difluorocyclohexyl)amino)-5-oxa-2-azaspiro[3.4]octan-2-yl)sulfonyl)-4-fluorobenzonitrile (56 mg, 31%) as a white solid. LCMS m/z=430.1 [M+H]⁺. ¹H NMR (500 MHz, MeOH-d₄): δ ppm 8.24 (dd, J=6.5, 2.5 Hz, 1H), 8.15-8.11 (m, 1H), 7.61 (t, J=9.0 Hz, 1H), 4.05 (d, J=8.5 Hz, 1H), 3.99-3.95 (m, 2H), 3.92-3.87 (m, 2H), 3.53-3.48 (m, 2H), 2.62-2.57 (m, 1H), 2.37-2.32 (m, 1H), 2.06-2.00 (m, 2H), 1.98-1.94 (m, 1H), 1.93-1.88 (m, 2H), 1.86-1.72 (m, 2H), 1.44-1.32 (m, 2H).

### Example 27

### 4-Fluoro-3-((7-((tetrahydro-2H-pyran-4-yl)amino)-5-oxa-2-azaspiro[3.4]octan-2-yl)sulfonyl)benzonitrile

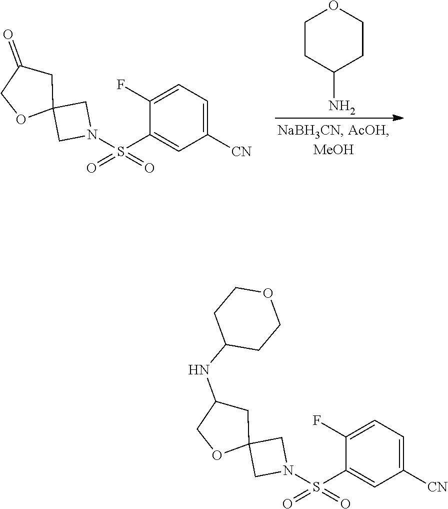 [SMILES: N#Cc1ccc(F)c(S(=O)(=O)N2CC3(CC(=O)CO3)C2)c1 ; NC1CCOCC1 ; N#Cc1ccc(F)c(S(=O)(=O)N2CC3(CC(NC4CCOCC4)CO3)C2)c1]

To a solution of 4-fluoro-3-((7-oxo-5-oxa-2-azaspiro[3.4]octan-2-yl)sulfonyl)benzonitrile (Example 26, step 1, 130 mg, 0.4 mmol) and tetrahydro-2H-pyran-4-amine (64 mg, 0.6 mmol) in MeOH (5 mL) was adjusted to pH=6 using AcOH and the solution stirred for 30 minutes at 20 °C. NaBH₃CN (132 mg, 2.1 mmol) was added and the reaction mixture was stirred at 20 °C for 14 h. The mixture was diluted with water (50 mL) and extracted with DCM (50 mL×3). The combined organic phase was dried over anhydrous sodium sulfate, filtered and concentrated in vacuum. The crude was purified by prep-HPLC ((Column: Boston Prime C18 150×30 mm×5 μm, Condition: water (NH₃·H₂O+NH₄HCO₃)-MeCN, 31% to 61%, Flow Rate (mL/min): 25)) to give 4-fluoro-3-((7-((tetrahydro-2H-pyran-4-yl)amino)-5-oxa-2-azaspiro[3.4]octan-2-yl)sulfonyl)benzonitrile (54 mg, 32%) as a white solid. LCMS m/z=396.0 [M+H]⁺. ¹H NMR (500 MHz, CDCl₃): δ ppm 8.20-8.17 (m, 1H), 7.92-7.88 (m, 1H), 7.39 (t, J=8.5 Hz, 1H), 4.09 (d, J=9.0 Hz, 1H), 4.00 (s, 3H), 3.99-3.94 (m, 3H), 3.93-3.90 (m, 1H), 3.59-3.53 (m, 2H), 3.41-3.34 (m, 2H), 2.67-2.60 (m, 1H), 2.27-2.25 (m, 1H), 2.09-2.05 (m, 1H), 1.82-1.78 (m, 2H), 1.50-1.27 (m, 2H).

### Example 28

### 3-((7-(2-Oxa-6-azaspiro[3.3]heptan-6-yl)-5-oxa-2-azaspiro[3.4]octan-2-yl)sulfonyl)-5-fluorobenzonitrile

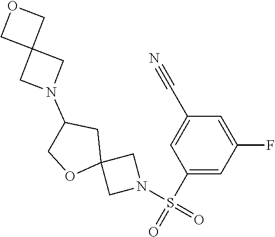 [SMILES: N#Cc1cc(F)cc(S(=O)(=O)N2CC3(CC(N4CC5(COC5)C4)CO3)C2)c1]

### 1. Synthesis of 3-fluoro-5-((7-oxo-5-oxa-2-azaspiro[3.4]octan-2-yl)sulfonyl)benzonitrile

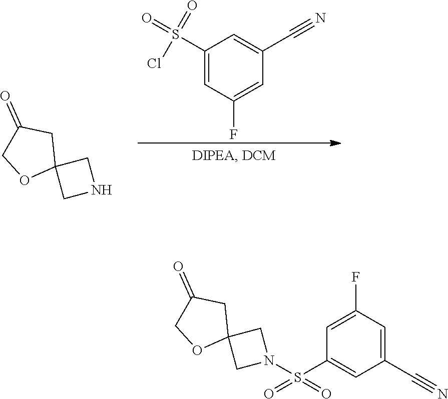 [SMILES: O=C1COC2(CNC2)C1 ; N#Cc1cc(F)cc(S(=O)(=O)Cl)c1 ; N#Cc1cc(F)cc(S(=O)(=O)N2CC3(CC(=O)CO3)C2)c1]

3-Fluoro-5-((7-oxo-5-oxa-2-azaspiro[3.4]octan-2-yl)sulfonyl)benzonitrile was obtained as a light yellow film (86 mg, 32%) from 5-oxa-2-azaspiro[3.4]octan-7-one HCl salt and 3-cyano-5-fluoro-benzenesulfonyl chloride following a similar procedure to that described in Example 18, step 1. ¹H NMR (500 MHz, DCM-d₂): δ ppm 7.96 (s, 1H), 7.81 (td, J=1.9, 7.6 Hz, 1H), 7.69 (td, J=1.2, 7.7 Hz, 1H), 4.04-4.01 (m, 2H), 3.97-3.94 (m, 4H), 2.72 (s, 2H).

### 2. Synthesis of 3-((7-(2-oxa-6-azaspiro[3.3]heptan-6-yl)-5-oxa-2-azaspiro[3.4]octan-2-yl)sulfonyl)-5-fluorobenzonitrile

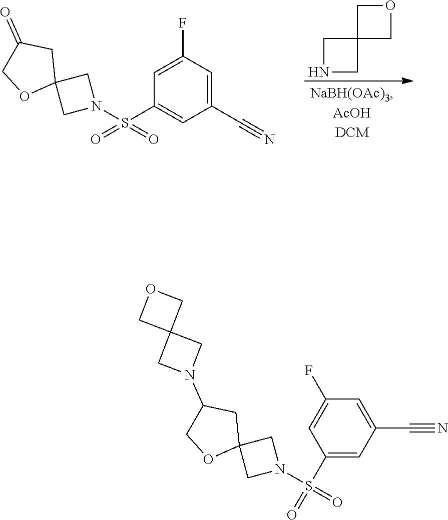 [SMILES: N#Cc1cc(F)cc(S(=O)(=O)N2CC3(CC(=O)CO3)C2)c1 ; C1NCC12COC2 ; N#Cc1cc(F)cc(S(=O)(=O)N2CC3(CC(N4CC5(COC5)C4)CO3)C2)c1]

To a vial containing 3-fluoro-5-((7-oxo-5-oxa-2-azaspiro[3.4]octan-2-yl) sulfonyl)benzonitrile (86 mg, 0.3 mmol) in anhydrous DCM (3 mL) was added AcOH (20 μL, 0.4 mmol) then 2-oxa-6-azaspiro[3.3]heptane (58 mg, 0.6 mmol) dropwise at 23 °C. After 15 mins, NaBH(OAc)₃ (223 mg, 1.1 mmol) was added in portions and the reaction was stirred at 23 °C for 1 h. The reaction was quenched by slow addition of aqueous 1 M NaOH solution, the mixture was stirred at 23 °C for 20 mins, then the biphasic mixture was extracted with DCM (3×). The combined organic extracts were dried over anhydrous MgSO₄, filtered and concentrated under reduced pressure. The residue was purified by silica gel column (50-100% 3:1 EtOAc:EtOH in heptane) to give 3-((7-(2-oxa-6-azaspiro[3.3]heptan-6-yl)-5-oxa-2-azaspiro[3.4]octan-2-yl)sulfonyl)-5-fluorobenzonitrile as a colorless film (25 mg, 22%). LCMS m/z=394.2 [M+H]⁺. ¹H NMR (500 MHz, DMSO-d₆): δ ppm 8.34 (br d, J=8.5 Hz, 1H), 8.22 (s, 1H), 8.13-8.10 (m, 1H), 4.56 (s, 4H), 3.89 (d, J=9.5 Hz, 1H), 3.82 (d, J=9.2 Hz, 1H), 3.76 (d, J=9.5 Hz, 1H), 3.65 (d, J=9.5 Hz, 1H), 3.50 (dd, J=9.0, 4.4 Hz, 1H), 3.39-3.36 (m, 1H), 3.31-3.29 (m, 1H), 3.21-3.15 (m, 4H), 2.80 (br s, 1H), 1.81-1.77 (m, 1H).

### Example 29

### 4-(2-((3,5-Dimethylpyridin-2-yl)sulfonyl)-2-azaspiro[3.4]octan-6-yl)morpholine

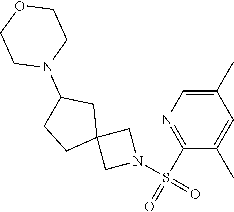 [SMILES: Cc1cnc(S(=O)(=O)N2CC3(CCC(N4CCOCC4)C3)C2)c(C)c1]

### 1. Synthesis of 2-azaspiro[3.4]octan-6-one

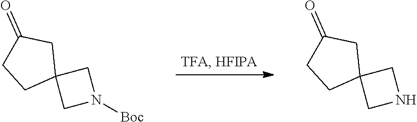 [SMILES: CC(C)(C)OC(=O)N1CC2(CCC(=O)C2)C1 ; O=C1CCC2(CNC2)C1]

To a solution of tert-butyl 6-oxo-2-azaspiro[3.4]octane-2-carboxylate (600 mg, 2.7 mmol) in HFIPA (20 mL) was added TFA (810 μL, 10.7 mmol) and the reaction was stirred for 2 h at 25 °C. The mixture was evaporated in vacuo to give 2-azaspiro[3.4]octan-6-one as TFA salt as a colorless oil and was used without further purification. ¹H NMR (400 MHz, MeOH-d₄): δ ppm 4.11-4.08 (m, 2H), 4.04-4.01 (m, 2H), 2.57 (s, 2H), 2.31 (s, 4H).

### 2. Synthesis of 2-((3,5-dimethylpyridin-2-yl)sulfonyl)-2-azaspiro[3.4]octan-6-one

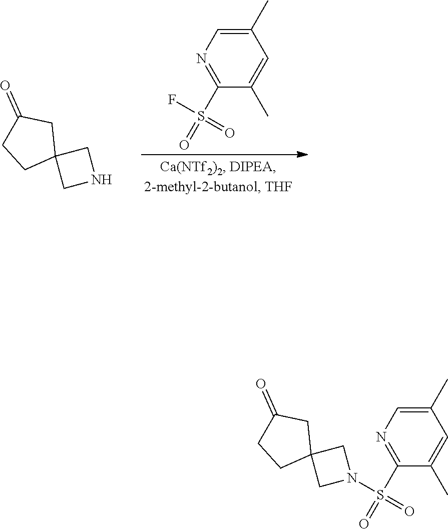 [SMILES: O=C1CCC2(CNC2)C1 ; Cc1cnc(S(=O)(=O)F)c(C)c1 ; Cc1cnc(S(=O)(=O)N2CC3(CCC(=O)C3)C2)c(C)c1]

To a solution of 2-azaspiro[3.4]octan-6-one (132 mg, 0.5 mmol, TFA salt) in anhydrous THF (1 mL) was added DIPEA (300 μL, 1.7 mmol) dropwise at rt, the solution stirred for 10 mins, then concentrated to afford a brown film. 2-Methyl-2-butanol (1 mL), 3,5-dimethylpyridine-2-sulfonyl fluoride (51 mg, 0.3 mmol) then Ca(NTf₂)₂ (169 mg, 0.3 mmol) were added in portions and the reaction was heated to 60 °C and stirred for 19 h. The reaction mixture was cooled to rt then quenched by slow addition of aqueous 2 M NaOH. The heterogeneous mixture was loaded onto a silica gel column and purified with (20-80% EtOAc in heptane) to afford 2-((3,5-dimethylpyridin-2-yl)sulfonyl)-2-azaspiro[3.4]octan-6-one as a colorless film (42 mg, 53%) that was used without further purification. LCMS m/z=295.0 [M+H]⁺.

### 3. Synthesis of 4-(2-((3,5-dimethylpyridin-2-yl)sulfonyl)-2-azaspiro[3.4]octan-6-yl)morpholine

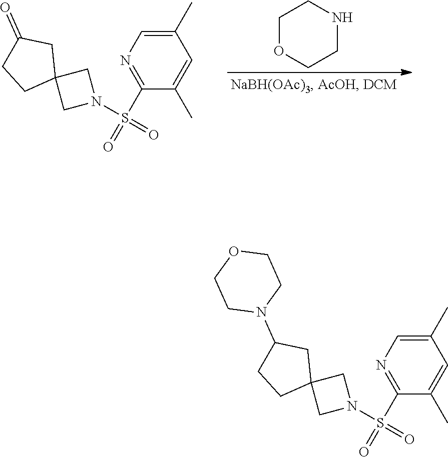 [SMILES: Cc1cnc(S(=O)(=O)N2CC3(CCC(=O)C3)C2)c(C)c1 ; C1COCCN1 ; Cc1cnc(S(=O)(=O)N2CC3(CCC(N4CCOCC4)C3)C2)c(C)c1]

To a solution of 2-((3,5-dimethylpyridin-2-yl)sulfonyl)-2-azaspiro[3.4]octan-6-one (42 mg, 0.1 mmol) in anhydrous DCM (1 mL) was added AcOH (10 μL, 0.2 mmol) then morpholine (30 μL, 0.3 mmol) dropwise at 23 °C. After 15 mins, NaBH(OAc)₃ (116 mg, 0.6 mmol) was added in portions and the reaction was stirred at 23 °C for 2 h. The reaction was quenched by slow addition of aqueous 1 M NaOH solution, the mixture was stirred at 23 °C for 20 mins, then the biphasic mixture was extracted with DCM (3×). The combined organic extracts were dried over anhydrous magnesium sulfate, the mixture filtered and concentrated under reduced pressure. The residue was purified by silica gel column eluting with (20-100% 3:1 EtOAc:EtOH in heptane). The product was further purified by HPLC using a Waters XSelect CSH C18, 5 μm, 50 mm×100 mm column with mobile phase H₂O (A) and MeCN (B) and a gradient of 5-50% B (0.2% NH₄OH final v/v % modifier) with flow rate at 80 mL/min to afford 4-(2-((3,5-dimethylpyridin-2-yl)sulfonyl)-2-azaspiro[3.4]octan-6-yl)morpholine as a white solid (101 mg, 21%). LCMS m/z=366.1 [M+H]⁺. ¹H NMR (600 MHz, DMSO-d₆): δ ppm 8.39-8.36 (m, 1H), 7.76-7.73 (m, 1H), 4.05-4.02 (m, 2H), 4.00-3.96 (m, 4H), 3.95-3.93 (m, 2H), 2.48 (s, 3H), 2.36-2.35 (m, 3H), 2.06 (br dd, J=7.3, 12.4 Hz, 2H), 1.92-1.87 (m, 2H), 1.83-1.78 (m, 2H), 1.77-1.74 (m, 1H), 1.69-1.63 (m, 2H), 1.45-1.37 (m, 2H).

### Example 30

### 6-(2-((6-Methoxy-2-methylpyridin-3-yl)sulfonyl)-2-azaspiro[3.4]octan-6-yl)-2-oxa-6-azaspiro[3.3]heptane

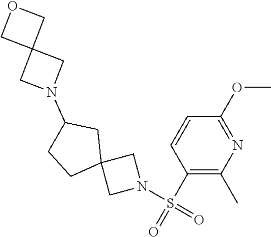 [SMILES: COc1ccc(S(=O)(=O)N2CC3(CCC(N4CC5(COC5)C4)C3)C2)c(C)n1]

### 1. Synthesis of 2-azaspiro[3.4]octan-6-one

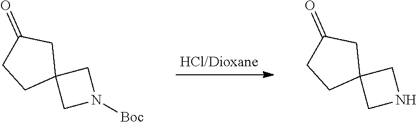 [SMILES: CC(C)(C)OC(=O)N1CC2(CCC(=O)C2)C1 ; O=C1CCC2(CNC2)C1]

A flask containing tert-butyl 6-oxo-2-azaspiro[3.4]octane-2-carboxylate (1.03 g, 4.6 mmol) in MeOH (10 mL) was cooled in an ice water bath, then 4M HCl in dioxane (4 M, 3.6 mL) was added dropwise. The reaction was allowed to warm to 23 °C and stirred for 6 h. The reaction was concentrated under reduced pressure, the residue was triturated with EtOAc and a couple of drops of MeOH added to afford 2-azaspiro[3.4]octan-6-one as HCl salt, as an off-white solid (721 mg, 97%) that was used without purification. ¹H NMR (500 MHz, DMSO-d₆): δ ppm 9.40-9.09 (m, 2H), 3.95-3.89 (m, 2H), 3.86-3.80 (m, 2H), 3.65-3.36 (m, 2H), 2.23-2.17 (m, 3H).

### 2. Synthesis of 2-((6-methoxy-2-methylpyridin-3-yl)sulfonyl)-2-azaspiro[3.4]octan-6-one

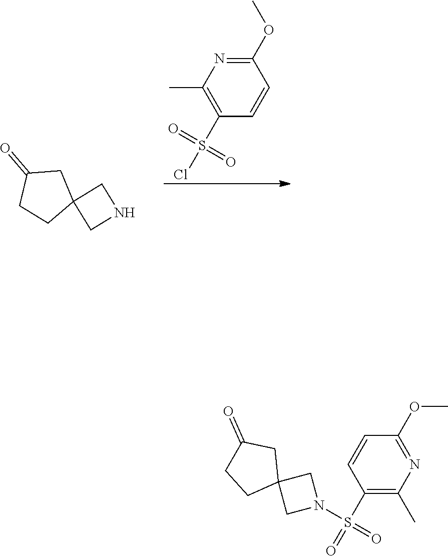 [SMILES: O=C1CCC2(CNC2)C1 ; COc1ccc(S(=O)(=O)Cl)c(C)n1 ; COc1ccc(S(=O)(=O)N2CC3(CCC(=O)C3)C2)c(C)n1]

To a solution of 2-azaspiro[3.4]octan-6-one (333 mg, 2.1 mmol, HCl salt) in anhydrous DCM (10 mL) was added DIPEA (1.5 mL, 8.6 mmol) dropwise at <5 °C and the solution stirred for 5 mins. 6-Methoxy-2-methyl-pyridine-3-sulfonyl chloride (577.1 mg, 2.60 mmol) was added and the reaction was allowed to warm to 23 °C and stirred for 30 mins. The reaction mixture was quenched by slow addition of aqueous 1 M NaOH solution. The mixture was stirred at 23 °C for 10 mins, then the biphasic mixture was loaded onto a silica gel column and purified with (10-55% EtOAc in heptane) to give 2-((6-methoxy-2-methylpyridin-3-yl)sulfonyl)-2-azaspiro[3.4]octan-6-one as a white solid (190 mg, 30% yield) that was used without further purification. LCMS m/z=311.1 [M+H]⁺.

### 3. Synthesis of 6-(2-((6-methoxy-2-methylpyridin-3-yl)sulfonyl)-2-azaspiro[3.4]octan-6-yl)-2-oxa-6-azaspiro[3.3]heptane

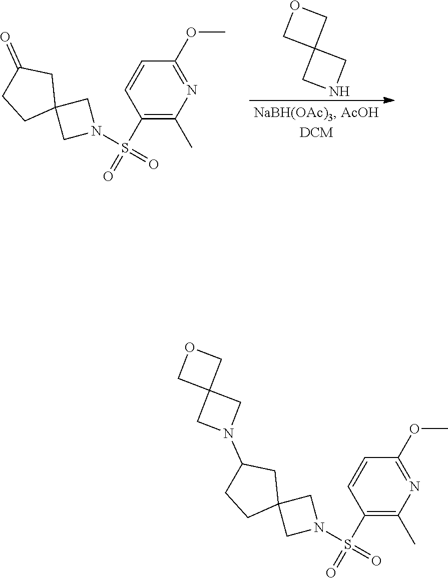 [SMILES: COc1ccc(S(=O)(=O)N2CC3(CCC(=O)C3)C2)c(C)n1 ; C1NCC12COC2 ; COc1ccc(S(=O)(=O)N2CC3(CCC(N4CC5(COC5)C4)C3)C2)c(C)n1]

6-(2-((6-Methoxy-2-methylpyridin-3-yl)sulfonyl)-2-azaspiro[3.4]octan-6-yl)-2-oxa-6-azaspiro[3.3]heptane was obtained as a colorless film (34 mg, 29%) from 2-((6-methoxy-2-methylpyridin-3-yl)sulfonyl)-2-azaspiro[3.4]octan-6-one (Example 30, step 2) and 2-oxa-6-azaspiro[3.3]heptane, following the procedure described in Example 29, step 2. LCMS m/z=394.2 [M+H]⁺. ¹H NMR (500 MHz, DMSO-d₆): δ ppm 8.02 (d, J=8.5 Hz, 1H), 6.85 (d, J=9.2 Hz, 1H), 4.54 (s, 4H), 3.93 (s, 3H), 3.60-3.54 (m, 4H), 3.12 (q, J=7.3 Hz, 4H), 2.67 (s, 3H), 2.54-2.52 (m, 1H), 1.79-1.72 (m, 1H), 1.66-1.59 (m, 2H), 1.52-1.41 (m, 2H), 1.27-1.21 (m, 1H).

### Example 31

### N-(4,4-Difluorocyclohexyl)-2-((6-methoxy-2-methylpyridin-3-yl)sulfonyl)-2-azaspiro[3.4]octan-6-amine

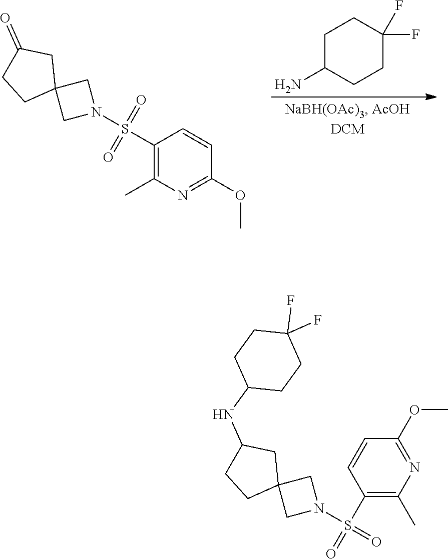 [SMILES: COc1ccc(S(=O)(=O)N2CC3(CCC(=O)C3)C2)c(C)n1 ; NC1CCC(F)(F)CC1 ; COc1ccc(S(=O)(=O)N2CC3(CCC(NC4CCC(F)(F)CC4)C3)C2)c(C)n1]

4,4-Difluorocyclohexanamine (165 mg, 1.0 mmol, HCl salt) was added to a solution of 2-((6-methoxy-2-methylpyridin-3-yl)sulfonyl)-2-azaspiro[3.4]octan-6-one (Example 30, step 2, 149 mg, 0.5 mmol) in DCM (3.1 mL) and the solution stirred at rt for 5 min. AcOH (230 μL, 3.8 mmol) was added dropwise, the solution stirred an additional 15 mins then NaBH(OAc)₃ (407 mg, 1.9 mmol) was added and the reaction stirred at rt for 3 days. The reaction was quenched by slow addition of aq. 1M NaOH solution, the mixture was stirred at ambient temperature for 20 min, then the biphasic mixture was extracted with DCM (3×). The combined organics were washed with water and brine then concentrated in vacuo. The crude was purified by silica gel column (0-100% EtOAc to 3:1 EtOAc:EtOH (w/2% NH₄OH)) to give N-(4,4-difluorocyclohexyl)-2-((6-methoxy-2-methylpyridin-3-yl)sulfonyl)-2-azaspiro[3.4]octan-6-amine as a white solid (126 mg, 61%). LCMS m/z=430.1 [M+H]⁺. ¹H NMR (500 MHz, DMSO-d₆): δ ppm 8.03 (d, J=9.2 Hz, 1H), 6.85 (d, J=9.2 Hz, 1H), 5.09 (br d, J=16.5 Hz, 1H), 3.98-3.89 (m, 3H), 3.69-3.52 (m, 4H), 3.09 (br s, 1H), 2.68 (s, 3H), 2.17 (br d, J=7.3 Hz, 1H), 2.02-1.85 (m, 3H), 1.85-1.67 (m, 6H), 1.66-1.60 (m, 1H), 1.46 (br dd, J=12.8, 6.1 Hz, 1H), 1.36-1.20 (m, 3H).

### Example 32

### 2-((6-Methoxy-2-methylpyridin-3-yl)sulfonyl)-N-(2-methoxyethyl)-N-methyl-2-azaspiro[3.4]octan-6-amine

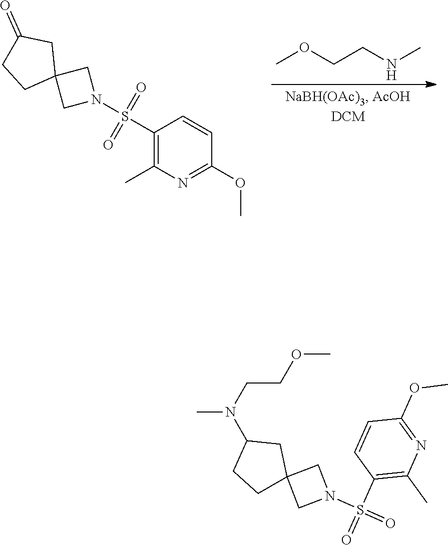

2-((6-Methoxy-2-methylpyridin-3-yl)sulfonyl)-N-(2-methoxyethyl)-N-methyl-2-azaspiro[3.4]octan-6-amine was obtained as an orange oil (75 mg, 41%), from 2-((6-methoxy-2-methylpyridin-3-yl)sulfonyl)-2-azaspiro[3.4]octan-6-one (Example 30, step 2) and 2-methoxy-N-methylethanamine, following the procedure described in Example 31. LCMS m/z=384.0 [M+H]^{+ 1}H NMR (500 MHz, DMSO-d₆): δ ppm 8.03 (d, J=9.2 Hz, 1H), 6.85 (d, J=8.5 Hz, 1H), 3.93 (s, 3H), 3.66-3.51 (m, 4H), 3.39-3.33 (m, 2H), 3.20 (s, 3H), 2.72-2.62 (m, 4H), 2.46-2.35 (m, 2H), 2.09 (br s, 3H), 1.94-1.82 (m, 1H), 1.79-1.61 (m, 3H), 1.55-1.46 (m, 1H), 1.38-1.29 (m, 1H).

### Example 33

### N-(2-Methoxyethyl)-N-methyl-2-((1-methyl-3-(trifluoromethyl)-1H-pyrazol-5-yl)sulfonyl)-2-azaspiro[3.4]octan-6-amine

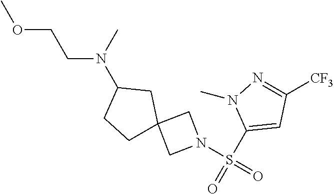 [SMILES: COCCN(C)C1CCC2(C1)CN(S(=O)(=O)c1cc(C(F)(F)F)nn1C)C2]

### 1. Synthesis of 2-((1-methyl-3-(trifluoromethyl)-1H-pyrazol-5-yl)sulfonyl)-2-azaspiro[3.4]octan-6-one

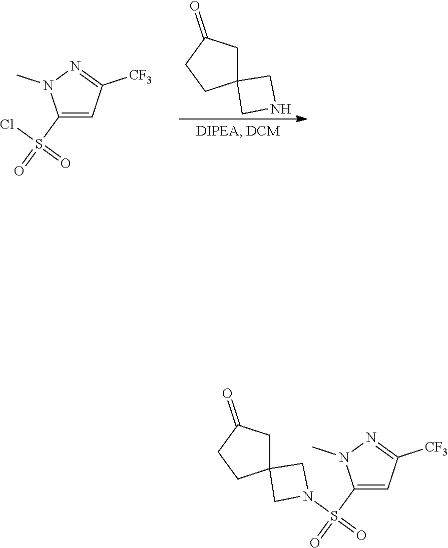 [SMILES: Cn1nc(C(F)(F)F)cc1S(=O)(=O)Cl ; O=C1CCC2(CNC2)C1 ; Cn1nc(C(F)(F)F)cc1S(=O)(=O)N1CC2(CCC(=O)C2)C1]

2-((1-Methyl-3-(trifluoromethyl)-1H-pyrazol-5-yl)sulfonyl)-2-azaspiro[3.4]octan-6-one was obtained, (161 mg 28%), from 2-azaspiro[3.4]octan-6-one HCl salt (Example 30, step 1) and 2-methyl-5-(trifluoromethyl)pyrazole-3-sulfonyl chloride, following a similar procedure to that described in Example 18, step 1. ¹H NMR (500 MHz, DCM-d₂): δ ppm 7.07-7.03 (m, 1H), 4.22-4.14 (m, 3H), 3.90-3.82 (m, 4H), 2.40 (s, 2H), 2.28-2.22 (m, 2H), 2.20-2.13 (m, 2H).

### 2. Synthesis of N-(2-methoxyethyl)-N-methyl-2-((1-methyl-3-(trifluoromethyl)-1H-pyrazol-5-yl)sulfonyl)-2-azaspiro[3.4]octan-6-amine

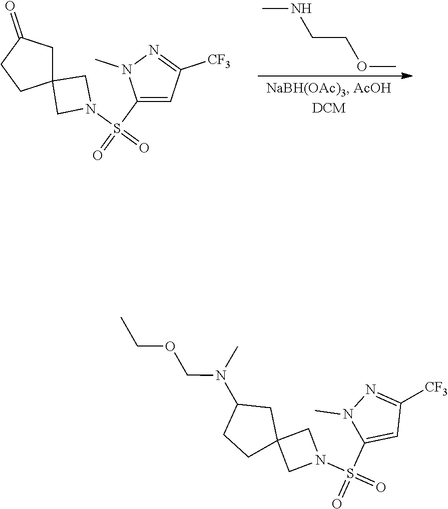

N-(2-Methoxyethyl)-N-methyl-2-((1-methyl-3-(trifluoromethyl)-1H-pyrazol-5-yl)sulfonyl)-2-azaspiro[3.4]octan-6-amine was obtained as a colorless film (50 mg, 47%) from 2-((1-methyl-3-(trifluoromethyl)-1H-pyrazol-5-yl)sulfonyl)-2-azaspiro[3.4]octan-6-one and 2-methoxy-N-methylethan-1-amine, following a similar procedure to that described in Example 29, step 3, except the crude product was purified by HPLC using a Waters XSelect CSH C18, 5 μm, 50 mm×100 mm column with mobile phase H₂O (A) and MeCN (B) and a gradient of 5-65% B (0.2% NH₄OH final v/v % modifier) at 60 mL/min. LCMS m/z=411.3 [M+H]⁺. ¹H NMR (500 MHz, DMSO-d₆): δ ppm 7.53 (s, 1H), 4.14 (s, 3H), 3.80-3.76 (m, 2H), 3.74-3.69 (m, 2H), 3.37-3.33 (m, 2H), 3.20 (s, 3H), 2.69-2.63 (m, 1H), 2.43-2.39 (m, 2H), 2.08 (s, 3H), 1.84-1.78 (m, 1H), 1.73-1.66 (m, 2H), 1.63-1.57 (m, 1H), 1.49-1.44 (m, 1H), 1.38-1.30 (m, 1H).

### Example 34

### 4-(2-((3-Methoxy-1-methyl-1H-pyrazol-5-yl)sulfonyl)-2-azaspiro[3.4]octan-6-yl)morpholine

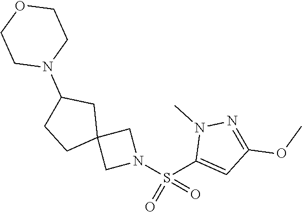 [SMILES: COc1cc(S(=O)(=O)N2CC3(CCC(N4CCOCC4)C3)C2)n(C)n1]

### 1. Synthesis of 2-((3-methoxy-1-methyl-1H-pyrazol-5-yl)sulfonyl)-2-azaspiro[3.4]octan-6-one

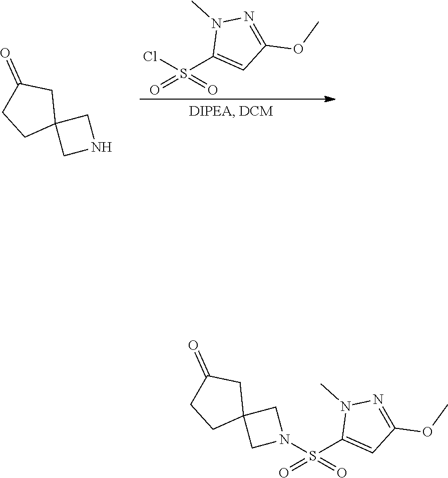 [SMILES: O=C1CCC2(CNC2)C1 ; COc1cc(S(=O)(=O)Cl)n(C)n1 ; COc1cc(S(=O)(=O)N2CC3(CCC(=O)C3)C2)n(C)n1]

2-((3-Methoxy-1-methyl-1H-pyrazol-5-yl)sulfonyl)-2-azaspiro[3.4]octan-6-one was obtained as a white solid (420 mg, 71%) from 2-azaspiro[3.4]octan-6-one (Example 29, Step 1) and 3-methoxy-1-methyl-1H-pyrazole-5-sulfonyl chloride, following a similar procedure to that described in Example 26, step 1. LCMS m/z=300.1 [M+H]⁺. ¹HNMR (400 MHz, CDCl₃) δ ppm 6.17 (s, 1H), 3.96 (s, 3H), 3.89 (s, 3H), 3.81 (s, 4H), 2.39 (s, 2H), 2.29-2.25 (m, 2H), 2.17-2.14 (m, 2H).

### 2. Synthesis of 4-(2-((3-methoxy-1-methyl-1H-pyrazol-5-yl)sulfonyl)-2-azaspiro[3.4]octan-6-yl)morpholine

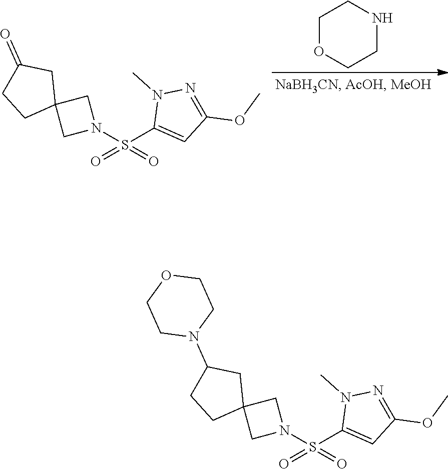 [SMILES: COc1cc(S(=O)(=O)N2CC3(CCC(=O)C3)C2)n(C)n1 ; C1COCCN1 ; COc1cc(S(=O)(=O)N2CC3(CCC(N4CCOCC4)C3)C2)n(C)n1]

A solution of 2-((3-methoxy-1-methyl-1H-pyrazol-5-yl)sulfonyl)-2-azaspiro[3.4]octan-6-one (200 mg, 0.668 mmol) and morpholine (90 μL, 1.0 mmol) in MeOH (10 mL) was adjusted to pH 5-6 with AcOH at 25 °C. The mixture was stirred at 25-30 °C for 0.5 h then NaBH₃CN (126 mg, 2.0 mmol) was added and the reaction was stirred at 25 °C for 1 h. The mixture was concentrated under reduced pressure and the residue was purified by prep-HPLC (Boston Green ODS 150×30 mm×5 μm, Condition: water (NH₄HCO₃)-MeCN, Flow Rate (mL/min) 25) to give 4-(2-((3-methoxy-1-methyl-1H-pyrazol-5-yl)sulfonyl)-2-azaspiro[3.4]octan-6-yl)morpholine (140 mg, 57%) as colorless oil. LCMS m/z=371.1 [M+H]⁺. ¹H NMR (400 MHz, CDCl₃) δ ppm 6.14 (s, 1H), 3.95 (s, 3H), 3.89 (s, 3H), 3.77-3.70 (m, 4H), 3.69-3.65 (m, 4H), 2.51-2.44 (m, 2H), 2.42-2.41 (m, 2H), 2.04-2.00 (m, 1H), 1.92-1.82 (m, 2H), 1.77-1.73 (m, 2H), 1.64-1.60 (m, 1H), 1.51-1.43 (m, 1H).

### Example 35

### 4-(2-((1-Methyl-3-(trifluoromethyl)-1H-pyrazol-5-yl)sulfonyl)-2-azaspiro[3.4]octan-6-yl)morpholine

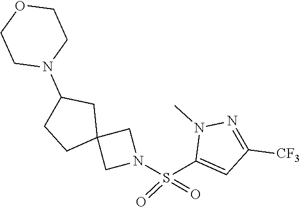 [SMILES: Cn1nc(C(F)(F)F)cc1S(=O)(=O)N1CC2(CCC(N3CCOCC3)C2)C1]

### 1. Synthesis of tert-butyl 6-morpholino-2-azaspiro[3.4]octane-2-carboxylate

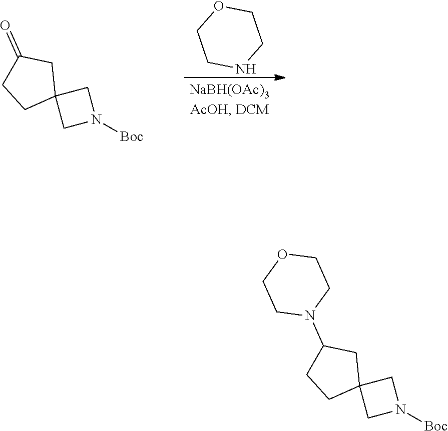 [SMILES: CC(C)(C)OC(=O)N1CC2(CCC(=O)C2)C1 ; C1COCCN1 ; CC(C)(C)OC(=O)N1CC2(CCC(N3CCOCC3)C2)C1]

To a vial containing tert-butyl 6-oxo-2-azaspiro[3.4]octane-2-carboxylate (904 mg, 4.0 mmol) in anhydrous DCM (20 mL) was added AcOH (250 μL, 4.2 mmol) then morpholine (730 μL, 8.4 mmol) was added dropwise at 23 °C and the solution stirred for 15 mins. NaBH(OAc)₃ (2.9 g, 13.7 mmol) was added in portions and the reaction was stirred at 23 °C for 7 h. The reaction was quenched by slow addition of aqueous 1 M NaOH solution, the mixture was stirred at 23 °C for 20 mins, then the biphasic mixture was extracted with DCM (3×). The combined organic extracts were dried over anhydrous magnesium sulfate, filtered and evaporated under reduced pressure to give tert-butyl 6-morpholino-2-azaspiro[3.4]octane-2-carboxylate (1.19 g, crude) that was used without further purification. LCMS m/z=297.1 [M+H]⁺

### 2. Synthesis of 4-(2-azaspiro[3.4]octan-6-yl)morpholine

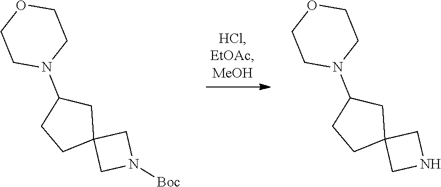 [SMILES: CC(C)(C)OC(=O)N1CC2(CCC(N3CCOCC3)C2)C1 ; C1CN(C2CCC3(CNC3)C2)CCO1]

A solution of tert-butyl 6-morpholin-4-yl-2-azaspiro[3.4]octane-2-carboxylate (1.19 g, 4.0 mmol) in MeOH (2.50 mL) was cooled in an ice water bath, then 1 M HCl in EtOAc (1 M, 14 mL) was added dropwise. Upon complete addition, the mixture was allowed to warm to 23 °C and stirred at rt for 5 days. The reaction was concentrated under reduced pressure, the residue was triturated with heptane and EtOAc to afford 4-(2-azaspiro[3.4]octan-6-yl)morpholine as the HCl salt, as a gummy white solid that was used without purification. LCMS m/z=197.1 [M+H]⁺

### 3. Synthesis of 4-(2-((1-methyl-3-(trifluoromethyl)-1H-pyrazol-5-yl)sulfonyl)-2-azaspiro 3.4 octan-6-yl)morpholine

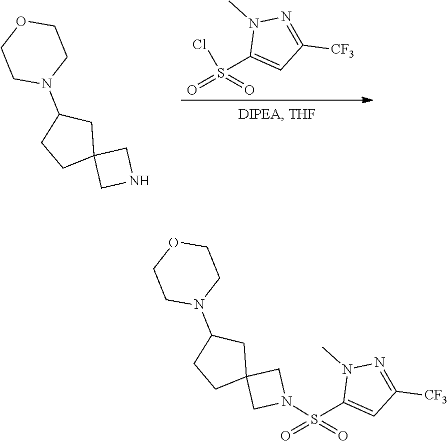 [SMILES: C1CN(C2CCC3(CNC3)C2)CCO1 ; Cn1nc(C(F)(F)F)cc1S(=O)(=O)Cl ; Cn1nc(C(F)(F)F)cc1S(=O)(=O)N1CC2(CCC(N3CCOCC3)C2)C1]

To a solution of 4-(2-azaspiro[3.4]octan-6-yl)morpholine (79 mg, 0.3 mmol, HCl salt) in anhydrous THE (2 mL) was added DIPEA (390 μL, 2.3 mmol) dropwise at <5 °C. After 5 mins, 2-methyl-5-(trifluoromethyl)pyrazole-3-sulfonyl chloride (124 mg, 0.5 mmol) was added to the cold solution and the reaction was allowed to warm to 23 °C and stirred for 30 mins. The reaction was quenched by slow addition of aqueous 1 M NaOH solution and the mixture was stirred at 23 °C for 10 mins. The biphasic mixture was loaded onto a silica gel column and purified with (15-75% 3:1 EtOAc:EtOH in heptane) to give 4-(2-((1-methyl-3-(trifluoromethyl)-1H-pyrazol-5-yl)sulfonyl)-2-azaspiro[3.4]octan-6-yl)morpholine as a colorless film (83 mg, 54%). LCMS m/z=409.2 [M+H]⁺. ¹H NMR (500 MHz, DMSO-d₆): δ ppm 7.53 (s, 1H), 4.14 (s, 3H), 3.77 (s, 2H), 3.75-3.71 (m, 2H), 3.54-3.50 (m, 4H), 2.46-2.39 (m, 1H), 2.34-2.21 (m, 4H), 1.87-1.83 (m, 1H), 1.76-1.68 (m, 2H), 1.65-1.59 (m, 1H), 1.52-1.47 (m, 1H), 1.39-1.30 (m, 1H).

### Example 36

### 4-(2-((1,3-Dimethyl-1H-pyrazol-5-yl)sulfonyl)-2-azaspiro[3.4]octan-6-yl)morpholine

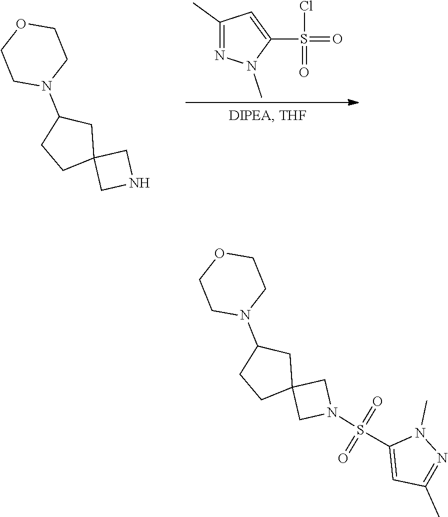 [SMILES: C1CN(C2CCC3(CNC3)C2)CCO1 ; Cc1cc(S(=O)(=O)Cl)n(C)n1 ; Cc1cc(S(=O)(=O)N2CC3(CCC(N4CCOCC4)C3)C2)n(C)n1]

4-(2-((1,3-Dimethyl-1H-pyrazol-5-yl)sulfonyl)-2-azaspiro[3.4]octan-6-yl)morpholine was obtained as a colorless film (43 mg, 33% yield) from 4-(2-azaspiro[3.4]octan-6-yl)morpholine (Example 35, step 2, HCl salt) and 2,5-dimethylpyrazole-3-sulfonyl chloride, following the procedure described in Example 35, step 3. LCMS m/z=355.2 [M+H]⁺. ¹H NMR (500 MHz, DMSO-d₆): δ ppm 6.70 (s, 1H), 3.96 (s, 3H), 3.66 (s, 2H), 3.64-3.60 (m, 2H), 3.52 (br t, J=4.6 Hz, 4H), 2.45-2.39 (m, 1H), 2.34-2.24 (m, 4H), 2.21 (s, 3H), 1.86-1.81 (m, 1H), 1.75-1.68 (m, 2H), 1.63-1.57 (m, 1H), 1.52-1.46 (m, 1H), 1.38-1.31 (m, 1H).

### Example 37

### 4-(2-((4-Chloro-1,3-dimethyl-1H-pyrazol-5-yl)sulfonyl)-2-azaspiro[3.4]octan-6-yl)morpholine

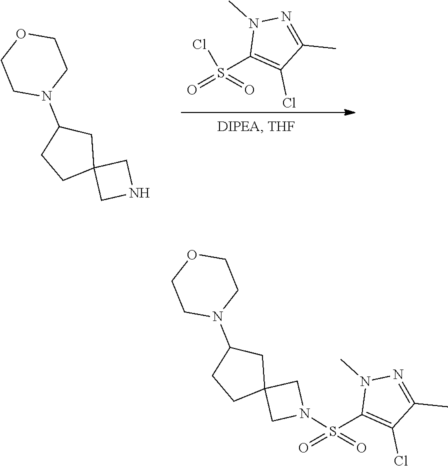 [SMILES: C1CN(C2CCC3(CNC3)C2)CCO1 ; Cc1nn(C)c(S(=O)(=O)Cl)c1Cl ; Cc1nn(C)c(S(=O)(=O)N2CC3(CCC(N4CCOCC4)C3)C2)c1Cl]

4-(2-((4-Chloro-1,3-dimethyl-1H-pyrazol-5-yl)sulfonyl)-2-azaspiro[3.4]octan-6-yl)morpholine was obtained as a light orange film (68 mg, 36%) from 4-(2-azaspiro[3.4]octan-6-yl)morpholine (Example 35, step 2, HCl salt) and 4-chloro-2,5-dimethylpyrazole-3-sulfonyl chloride, following the procedure described in Example 35, step 3. LCMS m/z=389.2 [M+H]⁺. ¹H NMR (500 MHz, DMSO-d6): δ ppm 3.99 (s, 3H), 3.78-3.74 (m, 2H), 3.74-3.69 (m, 2H), 3.52 (t, J=4.6 Hz, 4H), 2.47-2.40 (m, 1H), 2.34-2.24 (m, 4H), 2.21 (s, 3H), 1.87 (dd, J=12.8, 7.3 Hz, 1H), 1.78-1.68 (m, 2H), 1.67-1.61 (m, 1H), 1.54-1.49 (m, 1H), 1.40-1.32 (m, 1H).

### Example 38

### 4-(2-((4-(Difluoromethyl)-1,3-dimethyl-1H-pyrazol-5-yl)sulfonyl)-2-azaspiro[3.4]octan-6-yl)morpholine

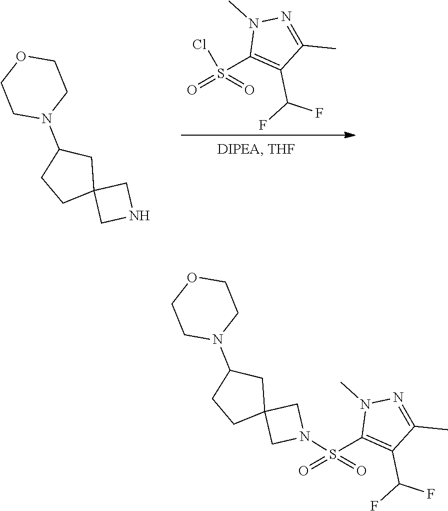 [SMILES: C1CN(C2CCC3(CNC3)C2)CCO1 ; Cc1nn(C)c(S(=O)(=O)Cl)c1C(F)F ; Cc1nn(C)c(S(=O)(=O)N2CC3(CCC(N4CCOCC4)C3)C2)c1C(F)F]

4-(2-((4-(Difluoromethyl)-1,3-dimethyl-1H-pyrazol-5-yl)sulfonyl)-2-azaspiro[3.4]octan-6-yl)morpholine was obtained as a light orange film (69 mg, 37%) from 4-(2-azaspiro[3.4]octan-6-yl)morpholine (Example 35, step 2, HCl salt) and 4-(difluoromethyl)-1,3-dimethyl-1H-pyrazole-5-sulfonyl chloride following the procedure described in Example 35, step 3. LCMS m/z=405.2 [M+H]⁺. ¹H NMR (500 MHz, DMSO-d₆): δ ppm 7.07 (t, J=54.0 Hz, 1H), 4.00 (s, 3H), 3.75-3.73 (m, 2H), 3.72-3.67 (m, 2H), 3.52 (t, J=4.6 Hz, 4H), 2.46-2.40 (m, 1H), 2.38-2.25 (m, 7H), 1.90 (dd, J=12.8, 6.7 Hz, 1H), 1.80-1.62 (m, 3H), 1.53 (dd, J=12.8, 8.5 Hz, 1H), 1.41-1.32 (m, 1H).

### Example 39

### 4-(2-((6-Methoxy-2-(trifluoromethyl)pyridin-3-yl)sulfonyl)-2-azaspiro[3.4]octan-6-yl)morpholine

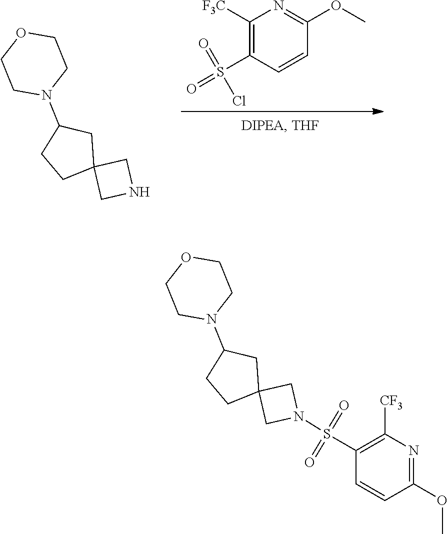 [SMILES: C1CN(C2CCC3(CNC3)C2)CCO1 ; COc1ccc(S(=O)(=O)Cl)c(C(F)(F)F)n1 ; COc1ccc(S(=O)(=O)N2CC3(CCC(N4CCOCC4)C3)C2)c(C(F)(F)F)n1]

4-(2-((6-Methoxy-2-(trifluoromethyl)pyridin-3-yl)sulfonyl)-2-azaspiro[3.4]octan-6-yl)morpholine was obtained as a colorless film (77 mg, 43%) from 4-(2-azaspiro[3.4]octan-6-yl)morpholine (Example 35 step 2, HCl salt) and 6-methoxy-2-(trifluoromethyl)pyridine-3-sulfonyl chloride, following the procedure described in Example 35, step 3. LCMS m/z=436.5 [M+H]⁺; ¹H NMR (500 MHz, DMSO-d₆): δ ppm 8.34 (d, J=8.5 Hz, 1H), 7.33 (d, J=8.5 Hz, 1H), 4.00 (s, 3H), 3.74-3.69 (m, 3H), 3.68-3.65 (m, 1H), 3.52 (t, J=4.6 Hz, 4H), 2.46-2.40 (m, 1H), 2.34-2.25 (m, 4H), 1.94 (dd, J=13.1, 7.0 Hz, 1H), 1.82-1.77 (m, 1H), 1.76-1.68 (m, 2H), 1.55 (dd, J=12.8, 9.2 Hz, 1H), 1.40-1.32 (m, 1H).

### Example 40

### 4-(2-((2-Methyl-6-(trifluoromethyl)pyridin-3-yl)sulfonyl)-2-azaspiro[3.4]octan-6-yl)morpholine

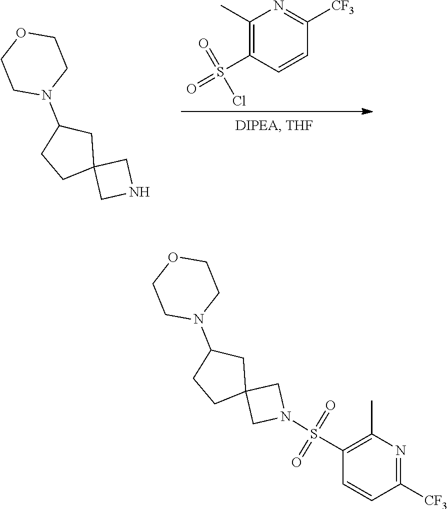 [SMILES: C1CN(C2CCC3(CNC3)C2)CCO1 ; Cc1nc(C(F)(F)F)ccc1S(=O)(=O)Cl ; Cc1nc(C(F)(F)F)ccc1S(=O)(=O)N1CC2(CCC(N3CCOCC3)C2)C1]

4-(2-((2-Methyl-6-(trifluoromethyl)pyridin-3-yl)sulfonyl)-2-azaspiro[3.4]octan-6-yl)morpholine was obtained as a colorless film (65 mg, 43%) from 4-(2-azaspiro[3.4]octan-6-yl)morpholine (Example 35, step 2, HCl salt) and 2-methyl-6-(trifluoromethyl)pyridine-3-sulfonyl chloride, following the procedure described in Example 35, step 3. LCMS m/z=420.5 [M+H]⁺. ¹H NMR (500 MHz, DMSO-d₆): δ ppm 8.45 (d, J=8.5 Hz, 1H), 7.99 (d, J=8.5 Hz, 1H), 3.78-3.73 (m, 3H), 3.72-3.69 (m, 1H), 3.52 (t, J=4.6 Hz, 4H), 2.83 (s, 3H), 2.47-2.41 (m, 1H), 2.36-2.22 (m, 4H), 1.96 (dd, J=12.8, 6.7 Hz, 1H), 1.85-1.79 (m, 1H), 1.77-1.68 (m, 2H), 1.59-1.54 (m, 1H), 1.41-1.32 (m, 1H).

### Example 41

### 4-(2-((4-Methyl-6-(trifluoromethyl)pyridin-3-yl)sulfonyl)-2-azaspiro[3.4]octan-6-yl)morpholine

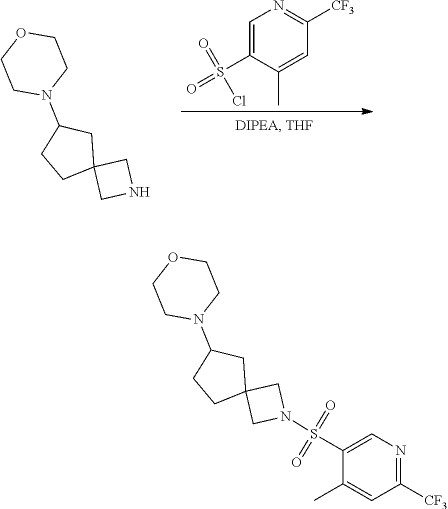 [SMILES: C1CN(C2CCC3(CNC3)C2)CCO1 ; Cc1cc(C(F)(F)F)ncc1S(=O)(=O)Cl ; Cc1cc(C(F)(F)F)ncc1S(=O)(=O)N1CC2(CCC(N3CCOCC3)C2)C1]

4-(2-((4-Methyl-6-(trifluoromethyl)pyridin-3-yl)sulfonyl)-2-azaspiro[3.4]octan-6-yl)morpholine was obtained as a yellow film (59 mg, 39%) from 4-(2-azaspiro[3.4]octan-6-yl)morpholine (Example 35, step 2, HCl salt) and 4-methyl-6-(trifluoromethyl)pyridine-3-sulfonyl chloride, following the procedure described in Example 35, step 3. LCMS m/z=420.2 [M+H]⁺. ¹H NMR (500 MHz, DMSO-d₆): δ ppm 9.03 (s, 1H), 8.14 (s, 1H), 3.78-3.75 (m, 2H), 3.75-3.69 (m, 2H), 3.52 (t, J=4.6 Hz, 4H), 2.70 (s, 3H), 2.47-2.41 (m, 1H), 2.35-2.25 (m, 4H), 1.96-1.90 (m, 1H), 1.82-1.77 (m, 1H), 1.76-1.67 (m, 2H), 1.58-1.52 (m, 1H), 1.40-1.33 (m, 1H).

### Example 42

### 4-(2-((6-Chloro-2-methylpyridin-3-yl)sulfonyl)-2-azaspiro[3.4]octan-6-yl)morpholine

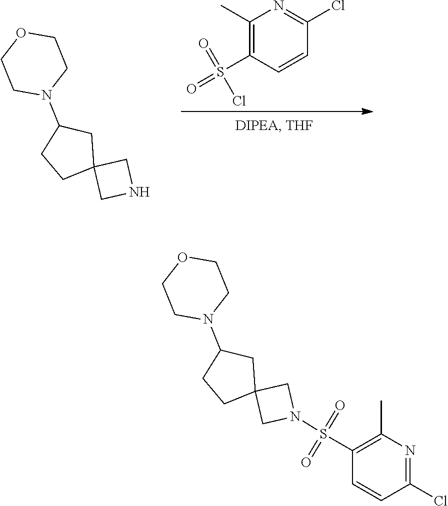 [SMILES: C1CN(C2CCC3(CNC3)C2)CCO1 ; Cc1nc(Cl)ccc1S(=O)(=O)Cl ; Cc1nc(Cl)ccc1S(=O)(=O)N1CC2(CCC(N3CCOCC3)C2)C1]

4-(2-((6-Chloro-2-methylpyridin-3-yl)sulfonyl)-2-azaspiro[3.4]octan-6-yl)morpholine was obtained as a light brown film (176 mg, 49%) from 4-(2-azaspiro[3.4]octan-6-yl)morpholine (Example 35, step 2, HCl salt) and 6-chloro-2-methylpyridine-3-sulfonyl chloride, following the procedure described in Example 35, step 3. LCMS m/z=386.1 [M+H]⁺. ¹H NMR (500 MHz, DMSO-d₆): δ ppm 8.18 (d, J=8.5 Hz, 1H), 7.61 (d, J=7.9 Hz, 1H), 3.70-3.62 (m, 4H), 3.52 (t, J=4.6 Hz, 4H), 2.73 (s, 3H), 2.47-2.41 (m, 1H), 2.35-2.26 (m, 4H), 1.94 (dd, J=12.8, 7.3 Hz, 1H), 1.81-1.76 (m, 1H), 1.75-1.67 (m, 2H), 1.57-1.51 (m, 1H), 1.40-1.33 (m, 1H).

### Example 43

### 4-(2-(Mesitylsulfonyl)-2-azaspiro[3.4]octan-6-yl)morpholine

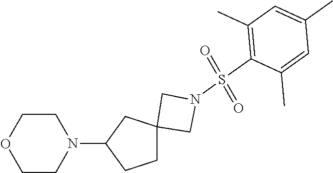 [SMILES: Cc1cc(C)c(S(=O)(=O)N2CC3(CCC(N4CCOCC4)C3)C2)c(C)c1]

### 1. Synthesis of 4-(2-azaspiro[3.4]octan-6-yl)morpholine

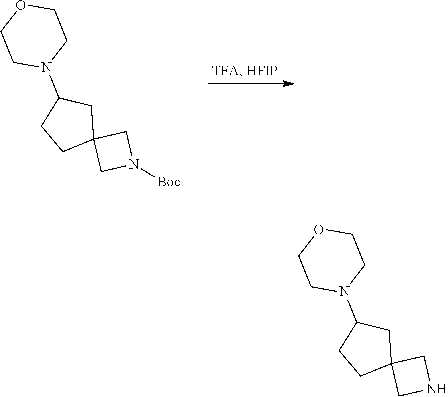 [SMILES: CC(C)(C)OC(=O)N1CC2(CCC(N3CCOCC3)C2)C1 ; C1CN(C2CCC3(CNC3)C2)CCO1]

A solution of tert-butyl 6-morpholin-4-yl-2-azaspiro[3.4]octane-2-carboxylate (Example 35, step 1, 918 mg, 3.1 mmol) in HFIP (9 mL) was cooled in an ice water bath, then TFA (840 μL, 11.0 mmol) was added dropwise. Upon complete addition, the reaction was allowed to warm to 23 °C and stirred for 7.5 h. The reaction was evaporated under reduced pressure to afford 4-(2-azaspiro[3.4]octan-6-yl)morpholine (as TFA salt) as a colorless film that was used without purification. LCMS m/z=197.1 [M+H]⁺.

### 2. Synthesis of 4-(2-(mesitylsulfonyl)-2-azaspiro[3.4]octan-6-yl)morpholine

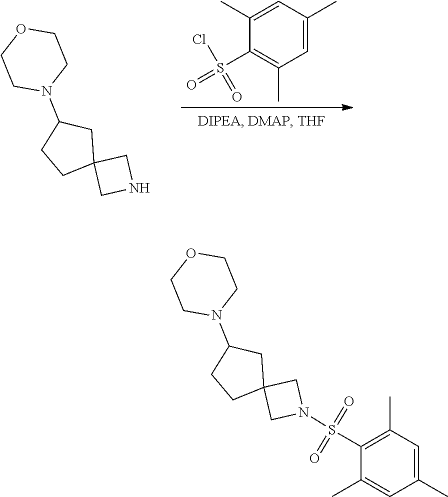 [SMILES: C1CN(C2CCC3(CNC3)C2)CCO1 ; Cc1cc(C)c(S(=O)(=O)Cl)c(C)c1 ; Cc1cc(C)c(S(=O)(=O)N2CC3(CCC(N4CCOCC4)C3)C2)c(C)c1]

To a solution of 4-(2-azaspiro[3.4]octan-6-yl)morpholine (144 mg, 0.5 mmol, TFA salt) in anhydrous THE (1.5 mL) was added DIPEA (0.5 mL, 2.9 mmol) dropwise and DMAP (6 mg, 0.1 mmol) at <5 °C. After 5 mins, 2,4,6-trimethylbenzenesulfonyl chloride (153 mg, 0.7 mmol) was added to the cold heterogeneous solution, the reaction was allowed to warm to 23 °C and stirred for 30 mins. The reaction was quenched by slow addition of aqueous 1 M NaOH solution, the mixture was stirred at 23 °C for 10 mins, then the biphasic mixture was extracted with EtOAc (3×). The combined organics were washed with saturated aqueous NaHCO₃ solution (2×) then dried over anhydrous sodium sulfate. The mixture was filtered and concentrated under reduced pressure. The residue was purified by silica gel column chromatography with (5-55% 3:1 EtOAc:EtOH in heptane) to give 4-(2-(mesitylsulfonyl)-2-azaspiro[3.4]octan-6-yl)morpholine (33 mg, 18%). LCMS m/z=379.2 [M+H]*; ¹H NMR (500 MHz, DMSO-d₆): δ ppm 7.07 (s, 2H), 3.63-3.56 (m, 3H), 3.55-3.50 (m, 5H), 2.53 (s, 6H), 2.47-2.41 (m, 1H), 2.41-2.28 (m, 4H), 2.27 (s, 3H), 1.97 (dd, J=12.8, 6.7 Hz, 1H), 1.84-1.78 (m, 1H), 1.77-1.68 (m, 2H), 1.56 (dd, J=12.8, 9.2 Hz, 1H), 1.42-1.33 (m, 1H).

### Example 44

### 4-(2-((4-(Difluoromethoxy)phenyl)sulfonyl)-2-azaspiro[3.4]octan-6-yl)morpholine

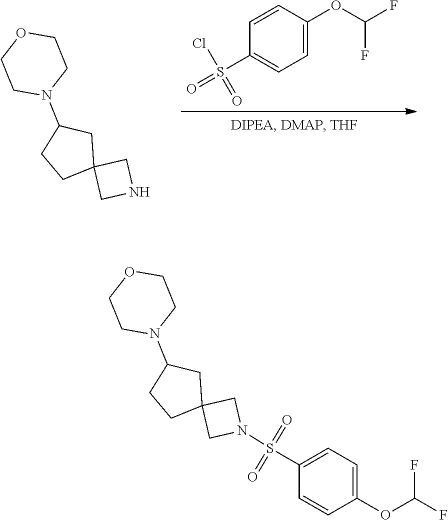 [SMILES: C1CN(C2CCC3(CNC3)C2)CCO1 ; O=S(=O)(Cl)c1ccc(OC(F)F)cc1 ; O=S(=O)(c1ccc(OC(F)F)cc1)N1CC2(CCC(N3CCOCC3)C2)C1]

4-(2-((4-(Difluoromethoxy)phenyl)sulfonyl)-2-azaspiro[3.4]octan-6-yl)morpholine was obtained as a TFA salt, as a colorless film (19 mg, 8%) from 4-(2-azaspiro[3.4]octan-6-yl)morpholine (TFA salt, Example 43, step 1 and 4-(difluoromethoxy)benzenesulfonyl chloride, following a similar procedure to that described in Example 43, step 2 except the product was additionally purified by prep HPLC using a Waters XSelect CSH C18, 5 m, 50 mm×100 mm column with mobile phase H₂O (A) and MeCN (B) and a gradient of 5-55% B (0.2% TFA final v/v % modifier). LCMS m/z=403.2 [M+H]⁺.

H NMR (500 MHz, DMSO-d₆) δ=8.04-7.83 (m, 2H), 7.65-7.30 (m, 2H), 4.03-3.91 (m, 1H), 3.73-3.41 (m, 4H), 3.40-3.25 (m, 2H), 3.06-2.92 (m, 1H), 2.62-2.41 (m, 3H), 2.31-2.15 (m, 1H), 2.13-2.00 (m, 1H), 1.97-1.84 (m, 1H), 1.82-1.50 (m, 4H), 1.50-1.21 (m, 2H).

### Example 45

### 4-(2-((2,4-Dimethylphenyl)sulfonyl)-2-azaspiro[3.4]octan-6-yl)morpholine

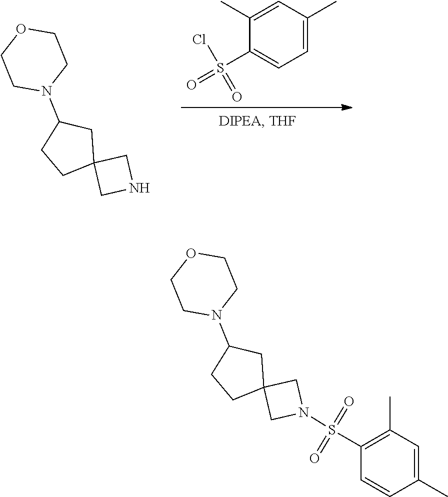 [SMILES: C1CN(C2CCC3(CNC3)C2)CCO1 ; Cc1ccc(S(=O)(=O)Cl)c(C)c1 ; Cc1ccc(S(=O)(=O)N2CC3(CCC(N4CCOCC4)C3)C2)c(C)c1]

4-(2-((2,4-Dimethylphenyl)sulfonyl)-2-azaspiro[3.4]octan-6-yl)morpholine was obtained as a colorless film (73 mg, 41%), from 4-(2-azaspiro[3.4]octan-6-yl)morpholine (TFA salt, Example 43, step 1) and 2,4-dimethylbenzenesulfonyl chloride, following a similar procedure to that described in Example 43, step 2, except DMAP was not used as a catalyst. LCMS m/z=365.1 [M+H]⁺. ¹H NMR (500 MHz, DMSO-d₆): δ ppm 7.70 (br d, J=7.9 Hz, 1H), 7.28 (br s, 1H), 7.24 (br d, J=7.9 Hz, 1H), 3.60-3.50 (m, 8H), 2.52 (br s, 3H), 2.42 (br s, 1H), 2.35 (br s, 3H), 2.29 (br s, 4H), 1.92-1.85 (m, 1H), 1.79-1.68 (m, 2H), 1.64 (br dd, J=11.3, 7.6 Hz, 1H), 1.50 (br t, J=10.4 Hz, 1H), 1.35 (br d, J=9.2 Hz, 1H).

### Example 46

### 4-(2-((2,4-Difluorophenyl)sulfonyl)-2-azaspiro[3.4]octan-6-yl)morpholine

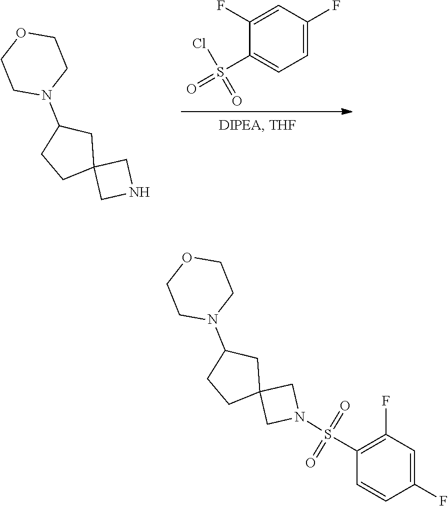 [SMILES: C1CN(C2CCC3(CNC3)C2)CCO1 ; O=S(=O)(Cl)c1ccc(F)cc1F ; O=S(=O)(c1ccc(F)cc1F)N1CC2(CCC(N3CCOCC3)C2)C1]

4-(2-((2,4-Difluorophenyl)sulfonyl)-2-azaspiro[3.4]octan-6-yl)morpholine was obtained as a colorless film (34 mg, 18%) from 4-(2-azaspiro[3.4]octan-6-yl)morpholine (TFA salt, Example 43, step 1) and 2,4-difluorobenzenesulfonyl chloride, following a similar procedure to that described in Example 35, step 3. The product was further purified by prep-HPLC using a Waters XSelect CSH C18, 5 m, 50 mm×100 mm column with mobile phase H₂O (A) and MeCN (B) and a gradient of 5-55% B (0.2% NH₄OH final v/v % modifier) with flow rate at 80 mL/min. LCMS m/z=373.2 [M+H]⁺. ¹H NMR (500 MHz, DMSO-d₆): δ ppm 7.89 (dt, J=8.4, 6.4 Hz, 1H), 7.71-7.64 (m, 1H), 7.38 (dt, J=8.4, 2.1 Hz, 1H), 3.78-3.49 (m, 7H), 2.45-2.12 (m, 2H), 1.88-1.65 (m, 5H), 1.64-1.29 (m, 5H).

### Example 47

### 4-(2-((4,6-Dimethylpyridin-3-yl)sulfonyl)-2-azaspiro[3.4]octan-6-yl)morpholine

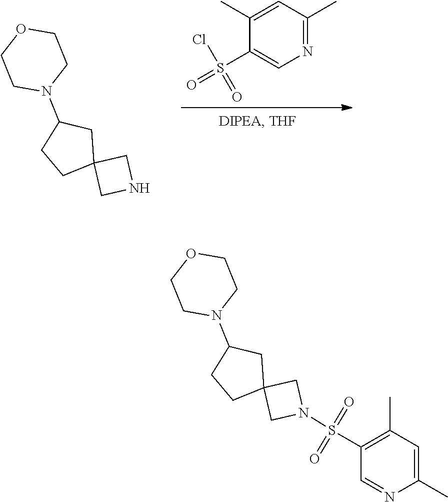 [SMILES: C1CN(C2CCC3(CNC3)C2)CCO1 ; Cc1cc(C)c(S(=O)(=O)Cl)cn1 ; Cc1cc(C)c(S(=O)(=O)N2CC3(CCC(N4CCOCC4)C3)C2)cn1]

4-(2-((4,6-Dimethylpyridin-3-yl)sulfonyl)-2-azaspiro[3.4]octan-6-yl)morpholine was obtained (22 mg, 19%) from 4-(2-azaspiro[3.4]octan-6-yl)morpholine (TFA salt, Example 43, step 1) and 4,6-dimethylpyridine-3-sulfonyl chloride, following a similar procedure to that described for Example 35, step 3, except the product was further purified by HPLC Waters XSelect CSH C18, 5 m, 50 mm×100 mm column with mobile phase H₂O (A) and MeCN (B) and a gradient of 5-45% B (0.2% NH₄OH final v/v % modifier) with flow rate at 80 mL/min. LCMS m/z=366.1 [M+H]⁺; ¹H NMR (500 MHz, DMSO-d₆): δ ppm 8.60 (s, 1H), 7.28 (s, 1H), 3.64-3.43 (m, 4H), 2.43-2.40 (m, 3H), 2.40-2.38 (m, 3H), 2.17 (br s, 4H), 1.78 (br dd, J=11.6, 7.9 Hz, 2H), 1.67-1.48 (m, 5H), 1.40 (br dd, J=12.5, 8.9 Hz, 2H), 1.28-1.18 (m, 2H).

### Example 48

### 4-(2-((6-methoxy-2-methylpyridin-3-yl)sulfonyl)-2-azaspiro[3.4]octan-6-yl)morpholine

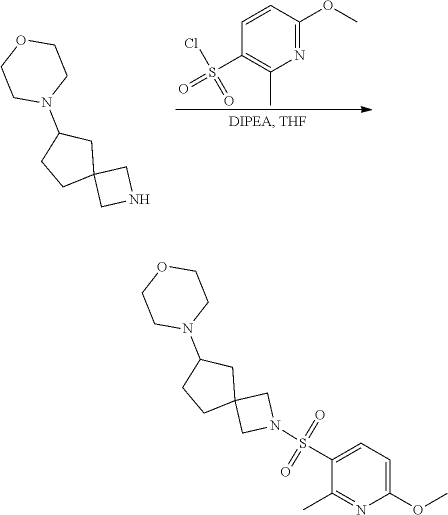 [SMILES: C1CN(C2CCC3(CNC3)C2)CCO1 ; COc1ccc(S(=O)(=O)Cl)c(C)n1 ; COc1ccc(S(=O)(=O)N2CC3(CCC(N4CCOCC4)C3)C2)c(C)n1]

4-(2-((6-Methoxy-2-methylpyridin-3-yl)sulfonyl)-2-azaspiro[3.4]octan-6-yl)morpholine was obtained as a colorless film (60 mg, 37%) from, 4-(2-azaspiro[3.4]octan-6-yl)morpholine (TFA salt, Example 43, step 1 and 6-methoxy-2-methylpyridine-3-sulfonyl chloride, following a similar procedure to that described for Example 35, step 3. LCMS m/z=382.5 [M+H]⁺. ¹H NMR (500 MHz, DMSO-d₆): δ ppm 8.03 (d, J=8.5 Hz, 1H), 6.85 (d, J=8.5 Hz, 1H), 3.93 (s, 3H), 3.64-3.58 (m, 3H), 3.56-3.51 (m, 5H), 2.68 (s, 3H), 2.45-2.39 (m, 1H), 2.34-2.24 (m, 4H), 1.91 (dd, J=12.8, 7.3 Hz, 1H), 1.80-1.65 (m, 3H), 1.52 (dd, J=12.8, 9.2 Hz, 1H), 1.40-1.31 (m, 1H).

### Example 49

### (R)-4-(2-((1-Methyl-3-(trifluoromethyl)-1H-pyrazol-5-yl)sulfonyl)-2-azaspiro[3.4]octan-6-yl)morpholine

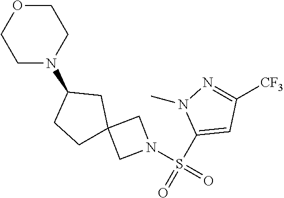 [SMILES: Cn1nc(C(F)(F)F)cc1S(=O)(=O)N1CC2(CC[C@@H](N3CCOCC3)C2)C1]

### 1. Synthesis of tert-butyl (R)-6-morpholino-2-azaspiro[3.4]octane-2-carboxylate

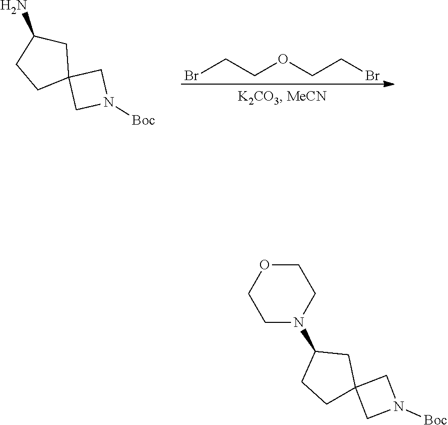 [SMILES: CC(C)(C)OC(=O)N1CC2(CC[C@@H](N)C2)C1 ; BrCCOCCBr ; CC(C)(C)OC(=O)N1CC2(CC[C@@H](N3CCOCC3)C2)C1]

To a solution of tert-butyl (R)-6-amino-2-azaspiro[3.4]octane-2-carboxylate (950 mg, 4.2 mmol) and 1-bromo-2-(2-bromoethoxy)ethane (1.48 g, 6.4 mmol) in MeCN (45 mL) was added K₂CO₃ (2.29 g, 16.6 mmol) in portions at 23 °C under N₂ and the reaction was heated to 90 °C for 19 h. The reaction was cooled to rt then filtered and the filtrate concentrated under reduced pressure. The residue was diluted with DCM then purified by silica gel column eluting with (10-65% 3:1 EtOAc:EtOH in heptane) to afford tert-butyl (R)-6-morpholino-2-azaspiro[3.4]octane-2-carboxylate as an oil (986 mg, 79%) that was used without purification. LCMS m/z=297.1 [M+H]⁺.

### 2. Synthesis of (R)-4-(2-azaspiro[3.4]octan-6-yl)morpholine

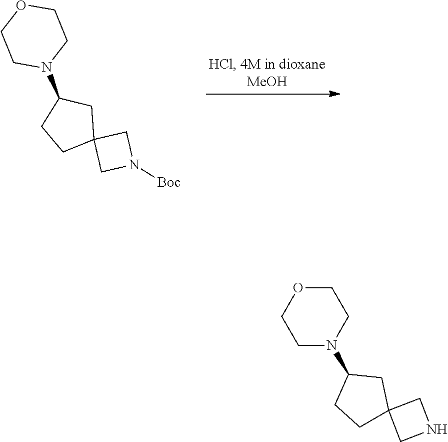 [SMILES: CC(C)(C)OC(=O)N1CC2(CC[C@@H](N3CCOCC3)C2)C1 ; C1CN([C@@H]2CCC3(CNC3)C2)CCO1]

A solution of tert-butyl (R)-6-morpholino-2-azaspiro[3.4]octane-2-carboxylate (986 mg, 3.3 mmol) in MeOH (4 mL) was cooled in an ice water bath, then 4M HCl in dioxane (4 M, 2.5 mL) was
added dropwise. The reaction was allowed to warm to 23 °C and stirred for 2 h, then evaporated under reduced pressure to give (R)-4-(2-azaspiro[3.4]octan-6-yl)morpholine as an HCl salt, that was used without purification. LCMS m/z=197.1 [M+H]⁺.

### 3. Synthesis of (R)-4-(2-((1-methyl-3-(trifluoromethyl)-1H-pyrazol-5-yl)sulfonyl)-2-azaspiro[3.4]octan-6-yl)morpholine

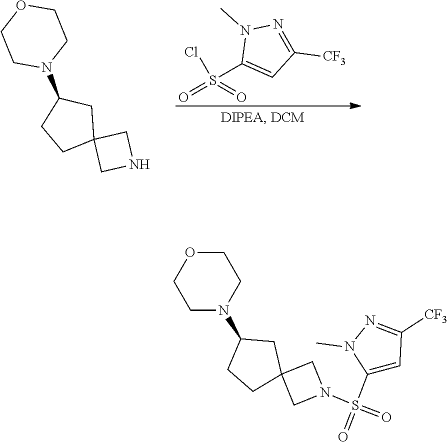 [SMILES: C1CN([C@@H]2CCC3(CNC3)C2)CCO1 ; Cn1nc(C(F)(F)F)cc1S(=O)(=O)Cl ; Cn1nc(C(F)(F)F)cc1S(=O)(=O)N1CC2(CC[C@@H](N3CCOCC3)C2)C1]

To a solution of 4-(2-azaspiro[3.4]octan-6-yl)morpholine (103 mg, 0.4 mmol) HCl salt, in anhydrous DCM (2 mL) was added DIPEA (410 μL, 2.4 mmol) dropwise under cooling in an ice water bath. After 5 mins, 2-methyl-5-(trifluoromethyl)pyrazole-3-sulfonyl chloride (111 mg, 0.5 mmol) was added and the reaction was allowed to warm to 23 °C and stirred for 30 mins. The reaction was quenched by slow addition of aqueous 1 M NaOH solution. The mixture was stirred at 23 °C for 20 minutes, then the biphasic mixture was extracted with DCM (3×). The combined organic extracts were dried over magnesium sulfate, the mixture filtered and concentrated under reduced pressure. The crude product was purified by silica gel column chromatography with (25-80% 3:1 EtOAc:EtOH in heptane.) The product was further purified by HPLC using a Waters XSelect CSH C18, 5 μm, 50 mm×100 mm column with mobile phase H₂O (A) and MeCN (B) and a gradient of 5-55% B (0.2% NH₄OH final v/v % modifier) with flow rate at 60 mL/min to give 4-(2-((1-methyl-3-(trifluoromethyl)-1H-pyrazol-5-yl)sulfonyl)-2-azaspiro[3.4]octan-6-yl)morpholine as a colorless film (57 mg, 30%). LCMS m/z=409.2 [M+H]⁺. ¹H NMR (500 MHz, MeOH-d₄): δ ppm 7.21 (s, 1H), 4.17 (s, 3H), 3.85-3.79 (m, 2H), 3.79-3.73 (m, 2H), 3.67 (t, J=4.6 Hz, 4H), 2.60-2.53 (m, 1H), 2.51-2.42 (m, 4H), 2.04 (dd, J=12.8, 7.3 Hz, 1H), 1.93-1.86 (m, 2H), 1.80-1.73 (m, 1H), 1.61 (dd, J=12.8, 9.2 Hz, 1H), 1.50-1.43 (m, 1H).

### Example 50

### (R)-3-Fluoro-5-((6-morpholino-2-azaspiro[3.4]octan-2-yl)sulfonyl)benzonitrile

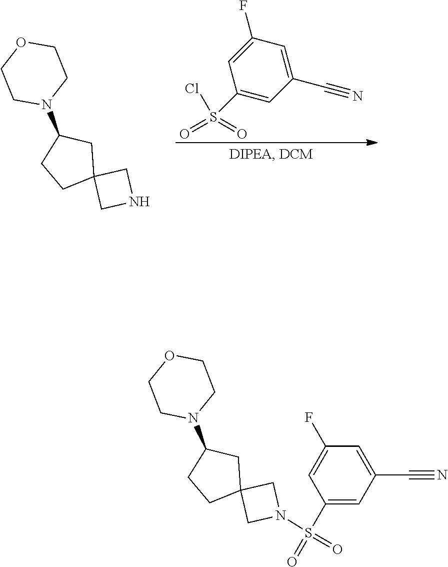 [SMILES: C1CN([C@@H]2CCC3(CNC3)C2)CCO1 ; N#Cc1cc(F)cc(S(=O)(=O)Cl)c1 ; N#Cc1cc(F)cc(S(=O)(=O)N2CC3(CC[C@@H](N4CCOCC4)C3)C2)c1]

(R)-3-Fluoro-5-((6-morpholino-2-azaspiro[3.4]octan-2-yl)sulfonyl)benzonitrile was obtained as a colorless film (29 mg, 18%) from (R)-4-(2-azaspiro[3.4]octan-6-yl)morpholine HCl salt (Example 49, step 2), and 3-cyano-5-fluoro-benzenesulfonyl chloride, following a similar procedure to that described in Example 49, step 3. LCMS m/z=380.2 [M+H]⁺. ¹H NMR (500 MHz, DMSO-d₆): δ ppm 8.33 (br d, J=8.2 Hz, 1H), 8.16 (s, 1H), 8.08-8.04 (m, 1H), 3.70-3.68 (m, 2H), 3.67-3.62 (m, 2H), 3.51 (br t, J=4.4 Hz, 4H), 2.40-2.34 (m, 1H), 2.32-2.17 (m, 4H), 1.74 (dd, J=7.3, 12.8 Hz, 1H), 1.69-1.59 (m, 2H), 1.54-1.47 (m, 1H), 1.40 (dd, J=8.5, 12.8 Hz, 1H), 1.35-1.26 (m, 1H).

### Example 51

### (S)-4-(2-((1-methyl-3-(trifluoromethyl)-1H-pyrazol-5-yl)sulfonyl)-2-azaspiro[3.4]octan-6-yl)morpholine

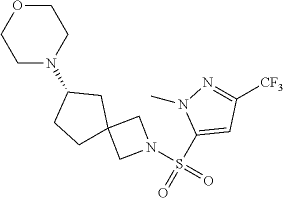 [SMILES: Cn1nc(C(F)(F)F)cc1S(=O)(=O)N1CC2(CC[C@H](N3CCOCC3)C2)C1]

### 1. Synthesis of tert-butyl (R)-6-((methylsulfonyl)oxy)-2-azaspiro[3.4]octane-2-carboxylate

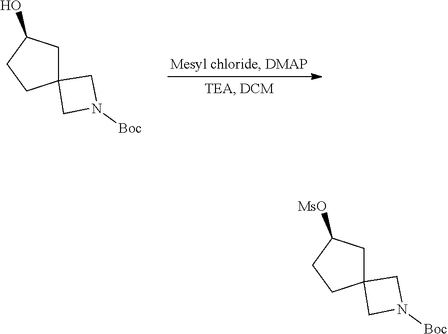 [SMILES: CC(C)(C)OC(=O)N1CC2(CC[C@@H](O)C2)C1 ; CC(C)(C)OC(=O)N1CC2(CC[C@@H](OS(C)(=O)=O)C2)C1]

A solution of tert-butyl (R)-6-hydroxy-2-azaspiro[3.4]octane-2-carboxylate (1.1 g, 4.9 mmol) in anhydrous DCM (40 mL) was cooled in an ice water bath then TEA (1.4 mL, 10.0 mmol) and DMAP (32 mg, 0.3 mmol) were added under cooling in an ice water bath and the solution stirred for 10 mins. Mesyl chloride (1.26 g, 11 mmol) was added dropwise, and the reaction was allowed to warm to rt and stirred for 5 h. The reaction was concentrated under reduced pressure, the residue was diluted with DCM then filtered. The filtrate was concentrated under reduced pressure and the crude was purified by silica gel column eluting with (5-80% EtOAc in heptane) to afford tert-butyl (R)-6-((methylsulfonyl)oxy)-2-azaspiro[3.4]octane-2-carboxylate as an oil (1.36 g, 91%) that was used without further purification. ¹H NMR (500 MHz, DCM-d2): δ ppm 5.10 (td, J=6.0, 2.8 Hz, 1H), 3.88 (d, J=8.5 Hz, 1H), 3.79-3.75 (m, 3H), 2.97 (s, 3H), 2.25-2.20 (m, 1H), 2.18-2.13 (m, 1H), 2.11-2.03 (m, 2H), 2.02-1.95 (m, 1H), 1.88-1.82 (m, 1H), 1.41 (s, 9H).

### 2. Synthesis of tert-butyl (S)-6-morpholino-2-azaspiro[3.4]octane-2-carboxylate

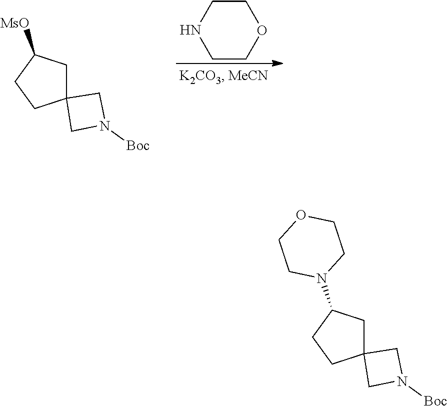 [SMILES: CC(C)(C)OC(=O)N1CC2(CC[C@@H](OS(C)(=O)=O)C2)C1 ; C1COCCN1 ; CC(C)(C)OC(=O)N1CC2(CC[C@H](N3CCOCC3)C2)C1]

To a flask containing morpholine (0.3 mL, 3.4 mmol) and tert-butyl (R)-6-methylsulfonyloxy-2-azaspiro[3.4]octane-2-carboxylate (1.36 g, 4.5 mmol) in anhydrous MeCN (22 mL) was added K₂CO₃ (1.21 g, 8.8 mmol) in portions at rt. Upon complete addition, the reaction was stirred at 95 °C for 28 h. The reaction was cooled to rt then stirred for a further 4 days. The reaction was concentrated under reduced pressure, the residue was diluted with DCM then filtered. The filtrate was concentrated under reduced pressure, then the residue was purified by silica gel column (10-65% 3:1 EtOAc:EtOH in heptane) to give tert-butyl (S)-6-morpholino-2-azaspiro[3.4]octane-2-carboxylate as an oil (463 mg, 46% yield). LCMS m/z=297.1 [M+H]⁺.

### 3. Synthesis of (S)-4-(2-azaspiro[3.4]octan-6-yl)morpholine

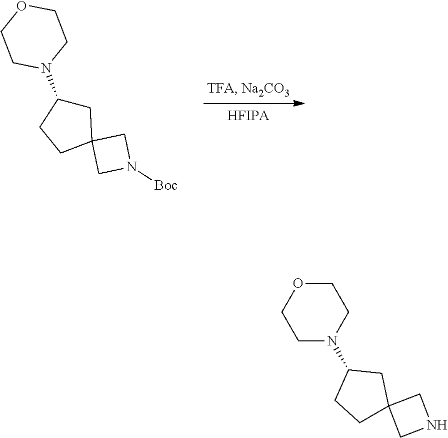 [SMILES: CC(C)(C)OC(=O)N1CC2(CC[C@H](N3CCOCC3)C2)C1 ; C1CN([C@H]2CCC3(CNC3)C2)CCO1]

A vial containing tert-butyl (6S)-6-morpholino-2-azaspiro[3.4]octane-2-carboxylate (463 mg, 1.6 mmol) in HFIPA (2 mL) was cooled in an ice water bath, then TFA (0.4 mL, 5.2 mmol) was added dropwise. Upon complete addition, the mixture was allowed to warm to 23 °C and stirred for 19 h. The reaction was concentrated under reduced pressure, the residue was dissolved in a mixture of DCM and MeOH (~9:1) and Na₂CO₃ (1.8 g, 17 mmol) added and the heterogeneous mixture stirred at rt for 2 h. The mixture was filtered and the filtrate was concentrated under reduced pressure to afford (S)-4-(2-azaspiro[3.4]octan-6-yl)morpholine as a light yellow film. LCMS m/z=197.1 [M+H]⁺.

### 4. Synthesis of(S)-4-(2-((1-methyl-3-(trifluoromethyl)-H-pyrazol-5-yl)sulfonyl)-2-azaspiro[3.4]octan-6-yl)morpholine

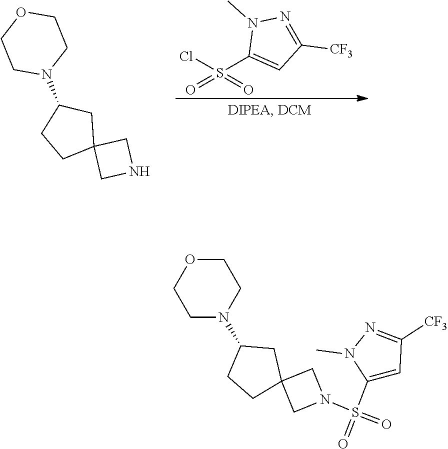 [SMILES: C1CN([C@H]2CCC3(CNC3)C2)CCO1 ; Cn1nc(C(F)(F)F)cc1S(=O)(=O)Cl ; Cn1nc(C(F)(F)F)cc1S(=O)(=O)N1CC2(CC[C@H](N3CCOCC3)C2)C1]

(S)-4-(2-((1-Methyl-3-(trifluoromethyl)-1H-pyrazol-5-yl)sulfonyl)-2-azaspiro[3.4]octan-6-yl)morpholine was obtained as a colorless film (12 mg, 6%) from (S)-4-(2-azaspiro[3.4]octan-6-yl)morpholine and 2-methyl-5-(trifluoromethyl)pyrazole-3-sulfonyl chloride, following a similar procedure to that described in Example 49, step 3. LCMS m/z=409.2 [M+H]⁺. ¹H NMR (500 MHz, DCM-d₂): δ ppm 7.02 (s, 1H), 4.14 (s, 3H), 3.82-3.79 (m, 1H), 3.77-3.70 (m, 3H), 3.65-3.60 (m, 4H), 2.51-2.46 (m, 1H), 2.44-2.34 (m, 4H), 1.99 (dd, J=12.8, 7.3 Hz, 1H), 1.91-1.81 (m, 2H), 1.78-1.72 (m, 1H), 1.66-1.61 (m, 1H), 1.50-1.43 (m, 1H).

### Example 52

### (S)-3-Fluoro-5-((6-morpholino-2-azaspiro[34]-octan-2-yl)sulfonyl)benzonitrile

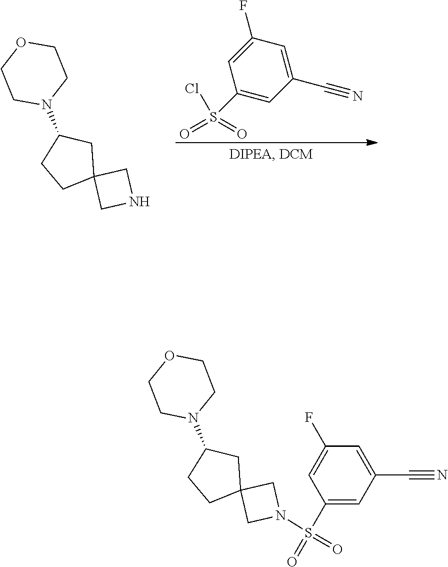 [SMILES: C1CN([C@H]2CCC3(CNC3)C2)CCO1 ; N#Cc1cc(F)cc(S(=O)(=O)Cl)c1 ; N#Cc1cc(F)cc(S(=O)(=O)N2CC3(CC[C@H](N4CCOCC4)C3)C2)c1]

(S)-3-Fluoro-5-((6-morpholino-2-azaspiro[3.4]octan-2-yl)sulfonyl)benzonitrile was obtained as a colorless film (14 mg, 11%) from (S)-4-(2-azaspiro[3.4]octan-6-yl)morpholine (Example 51, step 3) and 3-cyano-5-fluoro-benzenesulfonyl chloride, following a similar procedure described in Example 49, step 3, except the product was not additionally purified by HPLC. LCMS m/z=380.2 [M+H]⁺. ¹H NMR (500 MHz, DMSO-d₆): δ ppm 8.33 (br d, J=8.5 Hz, 1H), 8.16 (s, 1H), 8.05 (br d, J=7.9 Hz, 1H), 3.71-3.68 (m, 2H), 3.67-3.62 (m, 2H), 3.51 (t, J=4.4 Hz, 4H), 2.41-2.34 (m, 1H), 2.32-2.18 (m, 4H), 1.74 (dd, J=13.0, 7.2 Hz, 1H), 1.69-1.59 (m, 2H), 1.54-1.48 (m, 1H), 1.40 (dd, J=13.0, 8.7 Hz, 1H), 1.34-1.27 (m, 1H).

### Example 53

### 2-(Mesitylsulfonyl)-7-morpholino-5-oxa-2-azaspiro[3.4]octane

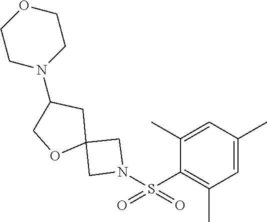 [SMILES: Cc1cc(C)c(S(=O)(=O)N2CC3(CC(N4CCOCC4)CO3)C2)c(C)c1]

### 1. Synthesis of tert-butyl 7-morpholino-5-oxa-2-azaspiro[3.4]octane-2-carboxylate

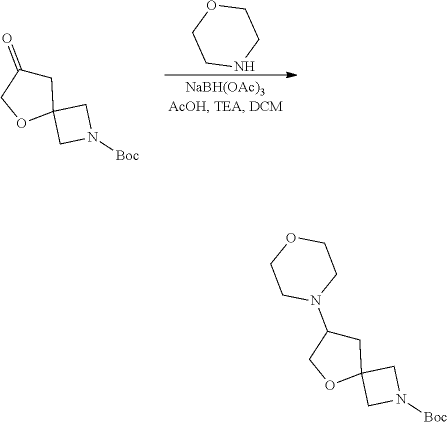 [SMILES: CC(C)(C)OC(=O)N1CC2(CC(=O)CO2)C1 ; C1COCCN1 ; CC(C)(C)OC(=O)N1CC2(CC(N3CCOCC3)CO2)C1]

To a solution of tert-butyl 7-oxo-5-oxa-2-azaspiro[3.4]octane-2-carboxylate (692 mg, 3.05 mmol) in anhydrous DCM (50 mL) was added morpholine (0.8 mL, 9.2 mmol) and TEA (480 μL, 3.4 mmol) dropwise at 23 °C. After 15 mins, AcOH (440 μL, 7.3 mmol) was added dropwise to the reaction mixture to pH-5-6. Upon complete addition, the reaction was stirred at 23 °C for 30 mins, then NaBH(OAc)₃ (5.33 g, 25.2 mmol) was added in portions. The reaction was stirred at 23 °C for 19 h (Additional anhydrous DCM (50 mL) was added to the thick white paste to aid stirring) then was quenched by slow addition of aqueous saturated ammonium chloride solution. The mixture was stirred at 23 °C for 15 mins, then the biphasic mixture was extracted with DCM (3×). The combined organic extracts were washed with brine (2×) then dried over anhydrous magnesium sulfate. The mixture was filtered and evaporated under reduced pressure to give tert-butyl (7S)-7-morpholino-5-oxa-2-azaspiro[3.4]octane-2-carboxylate that was used without purification. LCMS m/z=299.1 [M+H]⁺.

### 2. Synthesis of 7-morpholino-5-oxa-2-azaspiro[3.4]octane

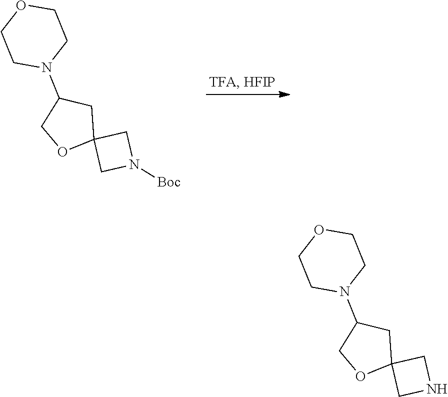 [SMILES: CC(C)(C)OC(=O)N1CC2(CC(N3CCOCC3)CO2)C1 ; C1CN(C2COC3(CNC3)C2)CCO1]

A solution of tert-butyl (7S)-7-morpholino-5-oxa-2-azaspiro[3.4]octane-2-carboxylate (908 mg, 3.0 mmol) in HFIP (7 mL) was cooled in an ice water bath, then TFA (0.7 mL, 9.1 mmol) was added dropwise. The mixture was allowed to warm to 23 °C and stirred for 8 h. The reaction was evaporated under reduced pressure to give 7-morpholino-5-oxa-2-azaspiro[3.4]octane (TFA salt) as a light yellow residue (950 mg) that was used without purification. LCMS m/z=199.1 [M+H]⁺.

### 3. Synthesis of 2-(mesitylsulfonyl)-7-morpholino-5-oxa-2-azaspiro[3.4]octane

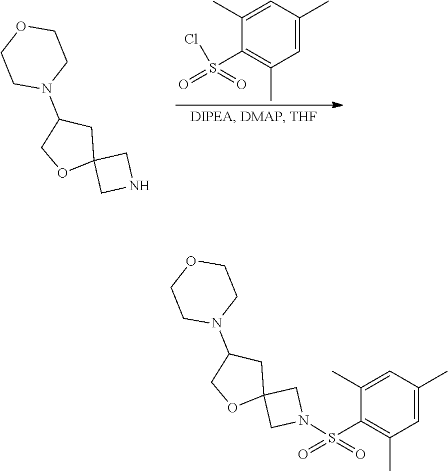 [SMILES: C1CN(C2COC3(CNC3)C2)CCO1 ; Cc1cc(C)c(S(=O)(=O)Cl)c(C)c1 ; Cc1cc(C)c(S(=O)(=O)N2CC3(CC(N4CCOCC4)CO3)C2)c(C)c1]

To a solution of 7-morpholino-5-oxa-2-azaspiro[3.4]octane (143 mg, 0.5 mmol, TFA salt) in anhydrous THE (1.5 mL) was added DIPEA (460 μL, 2.6 mmol) dropwise and DMAP (6 mg, 0.1 mmol) at <5 °C. After 5 mins, 2,4,6-trimethylbenzenesulfonyl chloride (147 mg, 0.7 mmol) was added to the cold heterogeneous solution, the reaction was allowed to warm to 23 °C and stirred for 30 mins. The reaction was quenched by slow addition of aqueous 1 M NaOH solution, the mixture was stirred at 23 °C for 10 mins, then the biphasic mixture was extracted with EtOAc (3×). The combined organics were washed with saturated aqueous NaHCO₃ solution (2×) then dried over anhydrous sodium sulfate. The mixture was filtered and concentrated under reduced pressure, and the residue was purified by silica gel column chromatography with (10-70% 3:1 EtOAc:EtOH in heptane) to give 2-(mesitylsulfonyl)-7-morpholino-5-oxa-2-azaspiro[3.4]octane as a colorless film (66 mg, 36%). LCMS m/z=381.2 [M+H]⁺. ¹H NMR (500 MHz, DMSO-d₆): δ ppm 7.08 (s, 2H), 3.82 (dd, J=8.5, 6.7 Hz, 1H), 3.77 (s, 2H), 3.71-3.64 (m, 2H), 3.58-3.51 (m, 5H), 2.90-2.83 (m, 1H), 2.53 (s, 6H), 2.36 (dt, J=4.0, 2.4 Hz, 2H), 2.31-2.24 (m, 6H), 2.02-1.97 (m, 1H).

### Example 54

### 2-((4-(Difluoromethoxy)phenyl)sulfonyl)-7-(4-methylpiperidin-1-yl)-5-oxa-2-azaspiro[3.4]octane

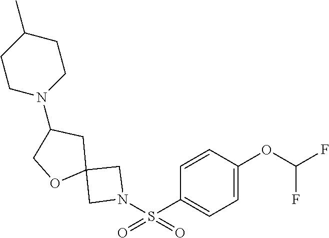 [SMILES: CC1CCN(C2COC3(C2)CN(S(=O)(=O)c2ccc(OC(F)F)cc2)C3)CC1]

### 1. Synthesis of tert-butyl 7-(4-methylpiperidin-1-yl)-5-oxa-2-azaspiro[3.4]octane-2-carboxylate

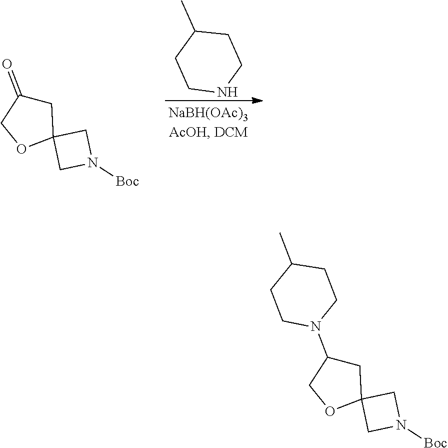

tert-Butyl 7-(4-methylpiperidin-1-yl)-5-oxa-2-azaspiro[3.4]octane-2-carboxylate was obtained (742 mg, 95%) from tert-butyl 7-oxo-5-oxa-2-azaspiro[3.4]octane-2-carboxylate and 4-methylpiperidine, following the procedure described in Example 35, step 1. LCMS m/z=311.2 [M+H]⁺

### 2. Synthesis of 7-(4-methylpiperidin-1-yl)-5-oxa-2-azaspiro[3.4]octane

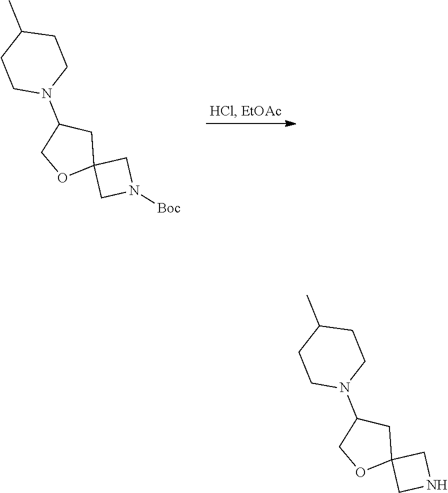 [SMILES: CC1CCN(C2COC3(C2)CN(C(=O)OC(C)(C)C)C3)CC1 ; CC1CCN(C2COC3(CNC3)C2)CC1]

7-(4-Methylpiperidin-1-yl)-5-oxa-2-azaspiro[3.4]octane was obtained as an HCl salt, as an off white solid, from tert-butyl 7-(4-methylpiperidin-1-yl)-5-oxa-2-azaspiro[3.4]octane-2-carboxylate, following the procedure described in Example 35, step 2. LCMS m/z=211.2 [M+H]⁺

### 3. Synthesis of 2-((4-(difluoromethoxy)phenyl)sulfonyl)-7-(4-methylpiperidin-1-yl)-5-oxa-2-azaspiro[3.4]octane

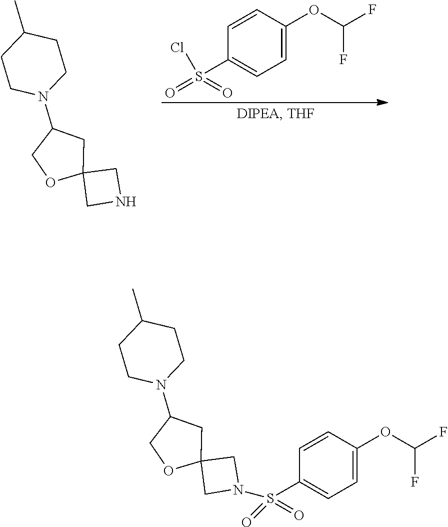 [SMILES: CC1CCN(C2COC3(CNC3)C2)CC1 ; O=S(=O)(Cl)c1ccc(OC(F)F)cc1 ; CC1CCN(C2COC3(C2)CN(S(=O)(=O)c2ccc(OC(F)F)cc2)C3)CC1]

2-((4-(Difluoromethoxy)phenyl)sulfonyl)-7-(4-methylpiperidin-1-yl)-5-oxa-2-azaspiro[3.4]octane was obtained as a colorless film (50 mg, 56%) from 7-(4-methylpiperidin-1-yl)-5-oxa-2-azaspiro[3.4]octane (HCl salt) and 4-(difluoromethoxy)benzenesulfonyl chloride following the procedure described in Example 35, step 3. LCMS m/z=417.5 [M+H]⁺. ¹H NMR (500 MHz, DMSO-d₆): δ ppm 7.92-7.86 (m, 2H), 7.64-7.32 (m, 3H), 3.83 (d, J=9.2 Hz, 1H), 3.78-3.70 (m, 2H), 3.63 (d, J=8.5 Hz, 1H), 3.51 (d, J=8.5 Hz, 1H), 3.40 (t, J=7.9 Hz, 1H), 2.78-2.71 (m, 1H), 2.71-2.66 (m, 1H), 2.56-2.51 (m, 1H), 2.09 (dd, J=12.8, 7.3 Hz, 1H), 1.87-1.75 (m, 3H), 1.51 (br t, J=14.6 Hz, 2H), 1.32-1.22 (m, 1H), 1.09-0.98 (m, 2H), 0.84 (d, J=6.7 Hz, 3H).

### Example 55

### 2-((1,3-Dimethyl-1H-pyrazol-5-yl)sulfonyl)-7-(4-methylpiperidin-1-yl)-5-oxa-2-azaspiro[3.4]octane

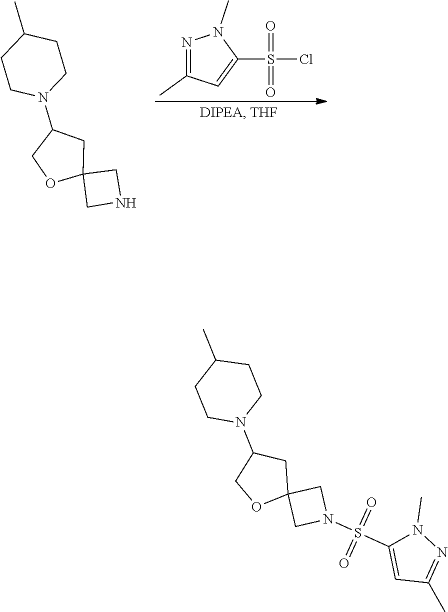 [SMILES: CC1CCN(C2COC3(CNC3)C2)CC1 ; Cc1cc(S(=O)(=O)Cl)n(C)n1 ; Cc1cc(S(=O)(=O)N2CC3(CC(N4CCC(C)CC4)CO3)C2)n(C)n1]

2-((1,3-Dimethyl-1H-pyrazol-5-yl)sulfonyl)-7-(4-methylpiperidin-1-yl)-5-oxa-2-azaspiro[3.4]octane was obtained as a colorless film (47 mg, 44%) from 7-(4-methylpiperidin-1-yl)-5-oxa-2-azaspiro[3.4]octane (HCl salt) (Example 54, step 2) and (1,3-dimethyl-1H-pyrazol-5-yl)sulfonyl chloride, following the procedure described in Example 35, step 3. LCMS m/z=369.2 [M+H]⁺. ¹H NMR (500 MHz, MeOH-d₄): δ ppm 6.65 (s, 1H), 4.01 (s, 3H), 3.96-3.89 (m, 3H), 3.85-3.81 (m, 1H), 3.78-3.74 (m, 1H), 3.61 (t, J=8.2 Hz, 1H), 2.98-2.87 (m, 2H), 2.73-2.66 (m, 1H), 2.40-2.34 (m, 1H), 2.28-2.25 (m, 3H), 2.08-1.94 (m, 3H), 1.70-1.62 (m, 2H), 1.45-1.36 (m, 1H), 1.26-1.16 (m, 2H), 0.93 (d, J=6.7 Hz, 3H).

### Example 56

### 2-((6-Methoxy-2-methylpyridin-3-yl)sulfonyl)-7-(4-methylpiperidin-1-yl)-5-oxa-2-azaspiro[3.4]octane

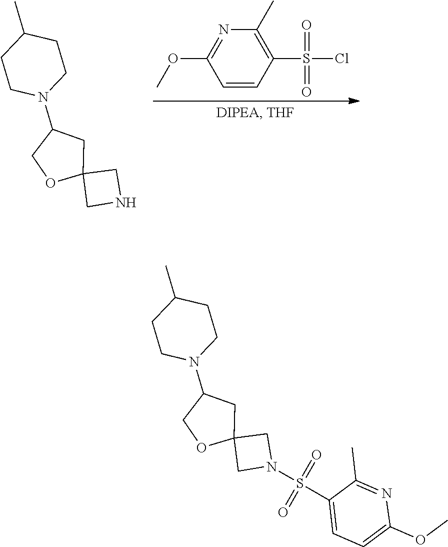 [SMILES: CC1CCN(C2COC3(CNC3)C2)CC1 ; COc1ccc(S(=O)(=O)Cl)c(C)n1 ; COc1ccc(S(=O)(=O)N2CC3(CC(N4CCC(C)CC4)CO3)C2)c(C)n1]

2-((6-Methoxy-2-methylpyridin-3-yl)sulfonyl)-7-(4-methylpiperidin-1-yl)-5-oxa-2-azaspiro[3.4]octane was obtained as a colorless film (11 mg, 9%) from 7-(4-methylpiperidin-1-yl)-5-oxa-2-azaspiro[3.4]octane (HCl salt) (Example 54, step 2) and 6-methoxy-2-methylpyridine-3-sulfonyl chloride, following the procedure described in Example 35, step 3. LCMS m/z=396.2 [M+H]⁺. ¹H NMR (500 MHz, MeOH-d₄): δ ppm 8.06 (d, J=8.5 Hz, 1H), 6.74 (d, J=9.2 Hz, 1H), 3.98 (s, 3H), 3.97-3.93 (m, 1H), 3.92-3.89 (m, 1H), 3.88-3.85 (m, 1H), 3.78-3.73 (m, 2H), 3.63 (t, J=7.9 Hz, 1H), 3.00-2.88 (m, 2H), 2.74-2.72 (m, 3H), 2.72-2.67 (m, 1H), 2.43 (dd, J=13.1, 7.6 Hz, 1H), 2.10-1.97 (m, 3H), 1.70-1.62 (m, 2H), 1.45-1.35 (m, 1H), 1.27-1.16 (m, 2H), 0.92 (d, J=6.7 Hz, 3H).

### Example 57

### (R)-2-((1-methyl-3-(trifluoromethyl)-1H-pyrazol-5-yl)sulfonyl)-7-morpholino-5-oxa-2-azaspiro[3.4]octane

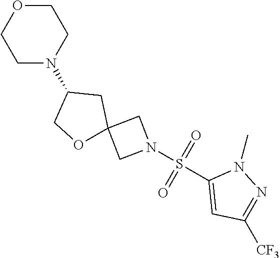 [SMILES: Cn1nc(C(F)(F)F)cc1S(=O)(=O)N1CC2(C[C@@H](N3CCOCC3)CO2)C1]

### 1. Synthesis of tert-butyl (S)-7-((methylsulfonyl)oxy)-5-oxa-2-azaspiro[3.4]octane-2-carboxylate

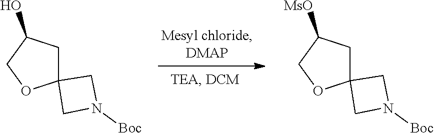 [SMILES: CC(C)(C)OC(=O)N1CC2(C[C@H](O)CO2)C1 ; CC(C)(C)OC(=O)N1CC2(C[C@H](OS(C)(=O)=O)CO2)C1]

tert-Butyl (S)-7-((methylsulfonyl)oxy)-5-oxa-2-azaspiro[3.4]octane-2-carboxylate was obtained as a white solid (1.28 g, 95%) from tert-butyl (S)-7-hydroxy-5-oxa-2-azaspiro[3.4]octane-2-carboxylate, following the procedure described in Example 51, step 1. LCMS m/z=308.0 [M+H]⁺

### 2. Synthesis of tert-butyl (R)-7-morpholino-5-oxa-2-azaspiro[3.4]octane-2-carboxylate

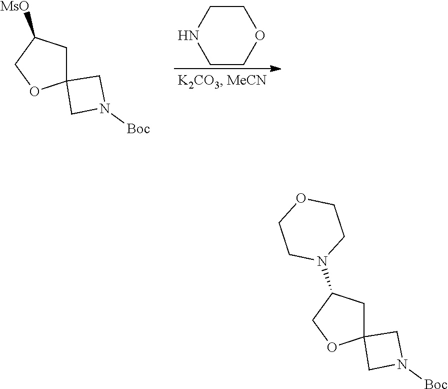 [SMILES: CC(C)(C)OC(=O)N1CC2(C[C@H](OS(C)(=O)=O)CO2)C1 ; C1COCCN1 ; CC(C)(C)OC(=O)N1CC2(C[C@@H](N3CCOCC3)CO2)C1]

tert-Butyl (R)-7-morpholino-5-oxa-2-azaspiro[3.4]octane-2-carboxylate was obtained as a white solid (124 mg, 11%) from tert-butyl (S)-7-((methylsulfonyl)oxy)-5-oxa-2-azaspiro[3.4]octane-2-carboxylate and morpholine, following the procedure described in Example 51, step 2. LCMS m/z=299.1 [M+H]⁺

### 3. Synthesis of (R)-7-morpholino-5-oxa-2-azaspiro[3.4]octane

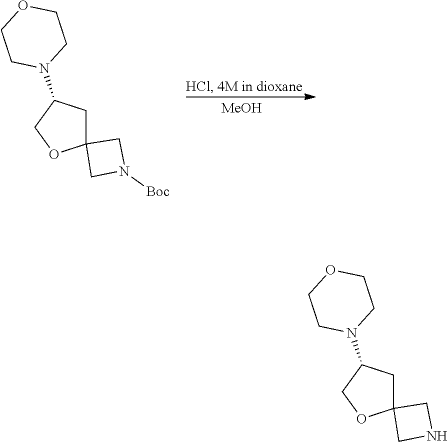 [SMILES: "MeOH" ; CC(C)(C)OC(=O)N1CC2(C[C@@H](N3CCOCC3)CO2)C1 ; C1CN([C@H]2COC3(CNC3)C2)CCO1]

A solution of tert-butyl (R)-7-morpholino-5-oxa-2-azaspiro[3.4]octane-2-carboxylate (124 mg, 0.4 mmol) in MeOH (1 mL) was cooled in an ice water bath, then 4M HCl in Dioxane (4 M, 0.32 mL) was added dropwise. The mixture was allowed to warm to 23 °C and stirred for 20 h. The reaction was filtered, rinsing with EtOAc to give (R)-7-morpholino-5-oxa-2-azaspiro[3.4]octane, as the hydrochloride salt, that was used without purification. LCMS m/z=199.1 [M+H]⁺.

### 4. Synthesis of (R)-2-((1-methyl-3-(trifluoromethyl)-H-pyrazol-5-yl)sulfonyl)-7-morpholino-5-oxa-2-azaspiro[3.4]octane

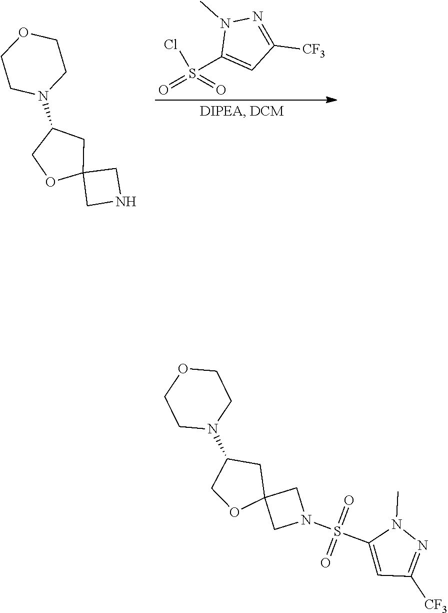 [SMILES: C1CN([C@H]2COC3(CNC3)C2)CCO1 ; Cn1nc(C(F)(F)F)cc1S(=O)(=O)Cl ; Cn1nc(C(F)(F)F)cc1S(=O)(=O)N1CC2(C[C@@H](N3CCOCC3)CO2)C1]

(R)-2-((1-Methyl-3-(trifluoromethyl)-1H-pyrazol-5-yl)sulfonyl)-7-morpholino-5-oxa-2-azaspiro[3.4]octane was obtained as a colorless film, from (R)-7-morpholino-5-oxa-2-azaspiro[3.4]octane (hydrochloride salt) and 2-methyl-5-(trifluoromethyl)pyrazole-3-sulfonyl chloride, following the procedure described in Example 51, step 4). LCMS m/z=411.1 [M+H]^{+ 1}H NMR (500 MHz, DCM-d₂): δ ppm 7.04 (s, 1H), 4.14 (s, 3H), 3.98 (s, 2H), 3.92-3.88 (m, 3H), 3.69-3.65 (m, 1H), 3.63 (t, J=4.9 Hz, 4H), 2.88 (quin, J=7.0 Hz, 1H), 2.45-2.40 (m, 2H), 2.34-2.27 (m, 3H), 2.07 (dd, J=12.8, 7.9 Hz, 1H).

### Example 58

### (R)-2-((2-Methyl-6-(trifluoromethyl)pyridin-3-yl)sulfonyl)-7-morpholino-5-oxa-2-azaspiro[3.4]octane

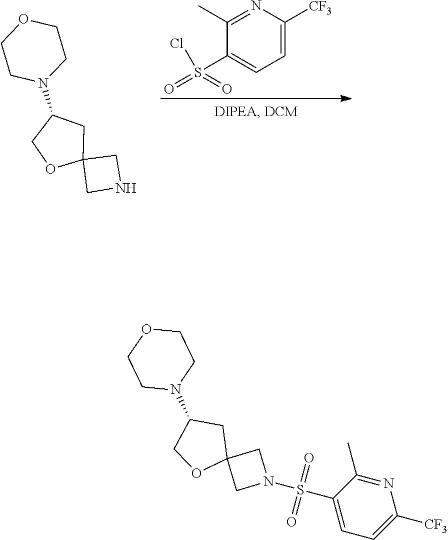 [SMILES: C1CN([C@H]2COC3(CNC3)C2)CCO1 ; Cc1nc(C(F)(F)F)ccc1S(=O)(=O)Cl ; Cc1nc(C(F)(F)F)ccc1S(=O)(=O)N1CC2(C[C@@H](N3CCOCC3)CO2)C1]

(R)-2-((2-Methyl-6-(trifluoromethyl)pyridin-3-yl)sulfonyl)-7-morpholino-5-oxa-2-azaspiro[3.4]octane was obtained as a colorless film (32 mg, 41%) from (R)-7-morpholino-5-oxa-2-azaspiro[3.4]octane (HCl salt) (Example 57, step 3) and 2-methyl-6-(trifluoromethyl)pyridine-3-sulfonyl chloride following the procedure described in Example 49, step 3. LCMS m/z=422.2 [M+H]⁺. ¹H NMR (400 MHz, DCM-d2): δ ppm 8.37 (d, J=8.0 Hz, 1H), 7.67 (d, J=8.3 Hz, 1H), 4.06 (d, J=8.3 Hz, 1H), 3.98-3.90 (m, 3H), 3.85 (d, J=8.3 Hz, 1H), 3.68 (dd, J=8.5, 7.0 Hz, 1H), 3.63 (t, J=4.6 Hz, 4H), 2.93-2.86 (m, 4H), 2.46-2.39 (m, 2H), 2.36-2.29 (m, 3H), 2.07 (dd, J=12.9, 7.9 Hz, 1H).

### Example 59

### 3-((7-((4,4-Difluorocyclohexyl)(methyl)amino)-5-oxa-2-azaspiro[3.4]octan-2-yl)sulfonyl)-4-fluorobenzonitrile

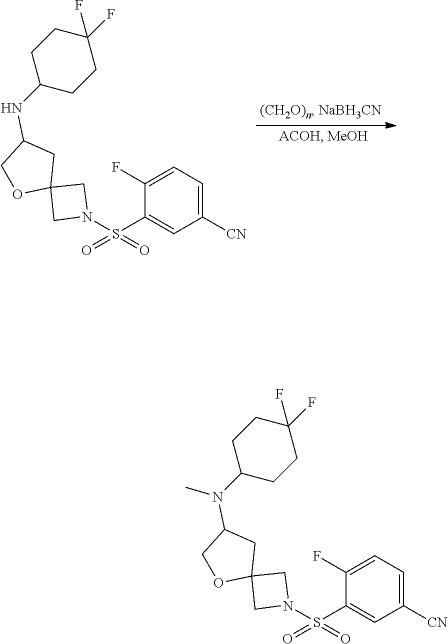 [SMILES: N#Cc1ccc(F)c(S(=O)(=O)N2CC3(CC(NC4CCC(F)(F)CC4)CO3)C2)c1 ; CN(C1CCC(F)(F)CC1)C1COC2(C1)CN(S(=O)(=O)c1cc(C#N)ccc1F)C2]

A solution of 3-((7-((4,4-difluorocyclohexyl)amino)-5-oxa-2-azaspiro[3.4]octan-2-yl)sulfonyl)-4-fluorobenzonitrile (Example 26, 90 mg, 0.2 mmol) and (CH₂O)ₙ(63 mg, 2.1 mmol) in MeOH (3 mL) was adjusted to pH=6 using AcOH. NaBH₃CN (65.85 mg, 1.05 mmol) was added and the reaction mixture was stirred at 20 °C for 14 h. The mixture was diluted with water (50 mL) and extracted with DCM (50 mL×3). The combined organic phase was dried over anhydrous sodium sulfate, filtered and concentrated in vacuo. The crude was purified by prep-HPLC ((Column: Boston Prime C18 150×30 mm×5 μm, Condition: water (NH₃·H₂O+NH₄HCO₃)-MeCN, 45% to 75%, Flow Rate (mL/min): 25)) to give 3-((7-((4,4-difluorocyclohexyl)(methyl)amino)-5-oxa-2-azaspiro[3.4]octan-2-yl)sulfonyl)-4-fluorobenzonitrile (40 mg, 43%) as a white solid. LCMS m/z=444.2 [M+H]⁺. ¹H NMR (400 MHz, MeOH-d₄): δ ppm 8.24 (dd, J=6.4, 2.0 Hz, 1H), 8.15-8.10 (m, 1H), 7.61 (t, J=9.6 Hz, 1H), 4.07-4.04 (m, 1H), 4.00-3.83 (m, 4H), 3.57 (t, J=8.0 Hz, 1H), 3.41-3.34 (m, 1H), 2.59 (t, J=11.6 Hz, 1H), 2.38-2.35 (m, 1H), 2.15 (s, 3H), 2.06-1.96 (m, 3H), 1.88-1.71 (m, 4H), 1.65-1.57 (m, 2H).

### Examples 60 and 61

### (R)-4-(2-((2-Methyl-6-(trifluoromethyl)pyridin-3-yl)sulfonyl)-2-azaspiro[3.4]octan-6-yl)morpholine and (S)-4-(2-((2-methyl-6-(trifluoromethyl)pyridin-3-yl)sulfonyl)-2-azaspiro[3.4]octan-6-yl morpholine

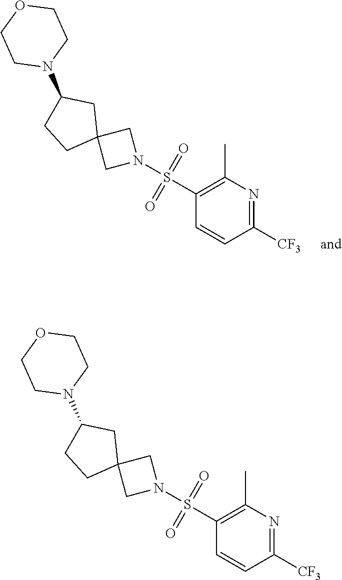 [SMILES: Cc1nc(C(F)(F)F)ccc1S(=O)(=O)N1CC2(CC[C@@H](N3CCOCC3)C2)C1 ; Cc1nc(C(F)(F)F)ccc1S(=O)(=O)N1CC2(CC[C@H](N3CCOCC3)C2)C1]

4-(2-((2-Methyl-6-(trifluoromethyl)pyridin-3-yl)sulfonyl)-2-azaspiro[3.4]octan-6-yl)morpholine (Example 40) was further purified by SFC using a Chiralpak IG 30×250 mm, 5 μm column using 20%

MeOH with 0.1% DMEA in CO₂; Flow rate: 100 mL/min; ABPR 120 bar; MBPR 40 psi, column temperature 40 °C; to give:

Peak 1, (R)-4-(2-((2-methyl-6-(trifluoromethyl)pyridin-3-yl)sulfonyl)-2-azaspiro[3.4]octan-6-yl)morpholine or (S)-4-(2-((2-methyl-6-(trifluoromethyl)pyridin-3-yl)sulfonyl)-2-azaspiro[3.4]octan-6-yl)morpholine as an off-white solid, (13 mg, 24%). LCMS m/z=420.2 [M+H]⁺. ¹H NMR (500 MHz, DMSO-d₆): δ ppm 8.48-8.43 (m, 1H), 8.00 (s, 1H), 3.79-3.74 (m, 3H), 3.72-3.69 (m, 1H), 3.54-3.51 (m, 4H), 2.83 (s, 3H), 2.48-2.41 (m, 1H), 2.37-2.27 (m, 4H), 1.99-1.93 (m, 1H), 1.85-1.78 (m, 1H), 1.77-1.68 (m, 2H), 1.59-1.53 (m, 1H), 1.41-1.34 (m, 1H).

Peak 2, (S)-4-(2-((2-methyl-6-(trifluoromethyl)pyridin-3-yl)sulfonyl)-2-azaspiro[3.4]octan-6-yl)morpholine or (R)-4-(2-((2-methyl-6-(trifluoromethyl)pyridin-3-yl)sulfonyl)-2-azaspiro[3.4]octan-6-yl)morpholine as an off-white solid (14 mg, 27%). LCMS m/z=420.2 [M+H]⁺. ¹H NMR (500 MHz, DMSO-d₆): δ ppm 8.45 (d, J=7.9 Hz, 1H), 7.99 (d, J=8.5 Hz, 1H), 3.79-3.73 (m, 3H), 3.72-3.69 (m, 1H), 3.52 (t, J=4.6 Hz, 4H), 2.83 (s, 3H), 2.46-2.41 (m, 1H), 2.38-2.27 (m, 4H), 1.96 (dd, J=12.8, 7.3 Hz, 1H), 1.84-1.78 (m, 1H), 1.76-1.68 (m, 2H), 1.59-1.53 (m, 1H), 1.41-1.33 (m, 1H).

Stereochemistry of the isomers was arbitrarily assigned.

### Examples 62 and 63

### (R)-4-(2-((4-Methyl-6-(trifluoromethyl)pyridin-3-yl)sulfonyl)-2-azaspiro[3.4]octan-6-yl)morpholine and (S)-4-(2-((4-methyl-6-(trifluoromethyl)pyridin-3-yl)sulfonyl)-2-azaspiro[3.4]octan-6-yl)morpholine

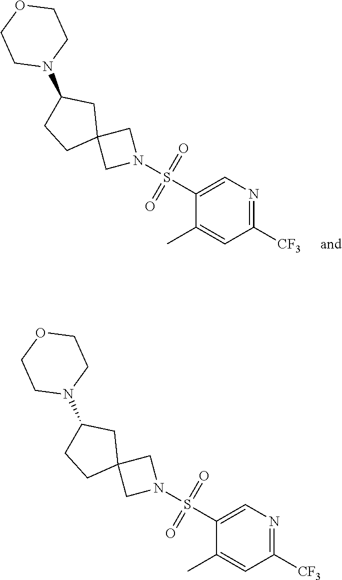 [SMILES: Cc1cc(C(F)(F)F)ncc1S(=O)(=O)N1CC2(CC[C@@H](N3CCOCC3)C2)C1 ; Cc1cc(C(F)(F)F)ncc1S(=O)(=O)N1CC2(CC[C@H](N3CCOCC3)C2)C1]

4-(2-((4-Methyl-6-(trifluoromethyl)pyridin-3-yl)sulfonyl)-2-azaspiro[3.4]octan-6-yl)morpholine (Example 41, 76 mg, 0.2 mmol) was further purified by chiral SFC using Chiralpak IA 30×250 mm, 5 μm column using 30% MeOH in CO₂. Flow rate: 100 mL/min; ABPR 120 bar; MBPR 40 psi, column temperature 40 °C to afford:

Peak 1, (R)-4-(2-((4-methyl-6-(trifluoromethyl)pyridin-3-yl)sulfonyl)-2-azaspiro[3.4]octan-6-yl)morpholine or (S)-4-(2-((4-methyl-6-(trifluoromethyl)pyridin-3-yl)sulfonyl)-2-azaspiro[3.4]octan-6-yl)morpholine as an off-white solid (21 mg, 26%). LCMS m/z=420.2 [M+H]⁺. ¹H NMR (500 MHz, MeOH-d₄): δ ppm 9.05 (s, 1H), 7.91 (s, 1H), 3.89-3.86 (m, 1H), 3.83-3.80 (m, 2H), 3.76-3.74 (m, 1H), 3.67 (t, J=4.6 Hz, 4H), 2.75 (s, 3H), 2.61-2.54 (m, 1H), 2.51-2.43 (m, 4H), 2.12 (dd, J=7.3, 12.8 Hz, 1H), 1.97-1.89 (m, 2H), 1.85-1.78 (m, 1H), 1.69-1.64 (m, 1H), 1.53-1.44 (m, 1H).

Peak 2, (S)-4-(2-((4-methyl-6-(trifluoromethyl)pyridin-3-yl)sulfonyl)-2-azaspiro[3.4]octan-6-yl)morpholine and (R)-4-(2-((4-methyl-6-(trifluoromethyl)pyridin-3-yl)sulfonyl)-2-azaspiro[3.4]octan-6-yl)morpholine as an off-white solid (22 mg, 28%). LCMS m/z=420.2 [M+H]⁺. ¹H NMR (500 MHz, MeOH-d₄): δ ppm 9.05 (s, 1H), 7.91 (s, 1H), 3.88-3.86 (m, 1H), 3.83-3.80 (m, 2H), 3.77-3.74 (m, 1H), 3.67 (t, J=4.6 Hz, 4H), 2.75 (s, 3H), 2.60-2.55 (m, 1H), 2.52-2.44 (m, 4H), 2.12 (dd, J=12.8, 7.3 Hz, 1H), 1.96-1.88 (m, 2H), 1.85-1.79 (m, 1H), 1.66 (dd, J=12.8, 9.2 Hz, 1H), 1.51-1.44 (m, 1H).

Stereochemistry of the isomers was arbitrarily assigned.

### Examples 64 and 65

### (R)-4-(2-((6-Chloro-2-methylpyridin-3-yl)sulfonyl)-2-azaspiro[3.4]octan-6-yl)morpholine and (S)-4-(2-((6-chloro-2-methylpyridin-3-yl)sulfonyl)-2-azaspiro[3.4]octan-6-yl)morpholine

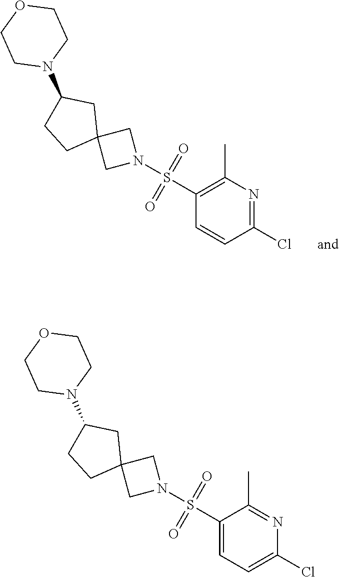 [SMILES: Cc1nc(Cl)ccc1S(=O)(=O)N1CC2(CC[C@@H](N3CCOCC3)C2)C1 ; Cc1nc(Cl)ccc1S(=O)(=O)N1CC2(CC[C@H](N3CCOCC3)C2)C1]

4-(2-((6-Chloro-2-methylpyridin-3-yl)sulfonyl)-2-azaspiro[3.4]octan-6-yl)morpholine (Example 42, 169 mg, 0.4 mmol) was further purified by SFC using Chiralpak IA 30×250 mm, 5 μm column using 30% MeOH with 0.1% DMEA in CO₂. Flow rate: 100 mL/min; ABPR 120 bar; MBPR 40 psi, column temperature 40 °C to afford: Peak 1, (R)-4-(2-((6-chloro-2-methylpyridin-3-yl)sulfonyl)-2-azaspiro[3.4]octan-6-yl)morpholine or (S)-4-(2-((6-chloro-2-methylpyridin-3-yl)sulfonyl)-2-azaspiro[3.4]octan-6-yl)morpholine as an off-white solid (51 mg, 28%). LCMS m/z=386.1 [M+H]⁺. ¹H NMR (500 MHz, MeOH-d4): δ ppm 8.21 (d, J=8.5 Hz, 1H), 7.48 (d, J=8.5 Hz, 1H), 3.83-3.80 (m, 1H), 3.77-3.74 (m, 2H), 3.71-3.67 (m, 5H), 2.79 (s, 3H), 2.62-2.55 (m, 1H), 2.53-2.45 (m, 4H), 2.12 (dd, J=12.8, 7.3 Hz, 1H), 1.95-1.88 (m, 2H), 1.84-1.78 (m, 1H), 1.66 (dd, J=12.8, 9.2 Hz, 1H), 1.51-1.45 (m, 1H).

Peak 2, (S)-4-(2-((6-chloro-2-methylpyridin-3-yl)sulfonyl)-2-azaspiro[3.4]octan-6-yl)morpholine or (R)-4-(2-((6-chloro-2-methylpyridin-3-yl)sulfonyl)-2-azaspiro[3.4]octan-6-yl)morpholine as an off-white solid (55 mg, 31%). LCMS m/z=386.1 [M+H]⁺. ¹H NMR (500 MHz, MeOH-d₄): δ ppm 8.21 (d, J=7.9 Hz, 1H), 7.48 (d, J=7.9 Hz, 1H), 3.83-3.80 (m, 1H), 3.77-3.75 (m, 2H), 3.71-3.67 (m, 5H), 2.79 (s, 3H), 2.60-2.54 (m, 1H), 2.52-2.45 (m, 4H), 2.12 (dd, J=13.1, 7.0 Hz, 1H), 1.96-1.88 (m, 2H), 1.84-1.78 (m, 1H), 1.66 (dd, J=13.1, 9.5 Hz, 1H), 1.51-1.44 (m, 1H).

Stereochemistry of the isomers was arbitrarily assigned.

### Examples 66 and 67

### (R)-4-(2-((1,3-dimethyl-1H-pyrazol-5-yl)sulfonyl)-2-azaspiro[3.4]octan-6-yl)morpholine and (S)-4-(2-((1,3-dimethyl-1H-pyrazol-5-yl)sulfonyl)-2-azaspiro[3.4]octan-6-yl)morpholine

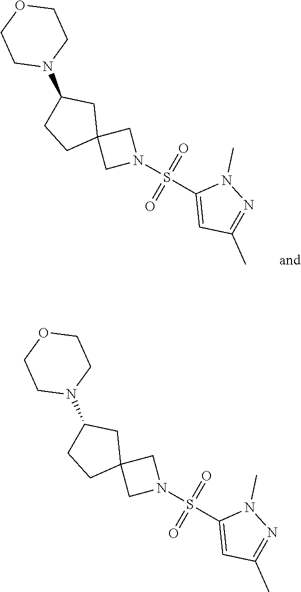 [SMILES: Cc1cc(S(=O)(=O)N2CC3(CC[C@@H](N4CCOCC4)C3)C2)n(C)n1 ; Cc1cc(S(=O)(=O)N2CC3(CC[C@H](N4CCOCC4)C3)C2)n(C)n1]

4-(2-((1,3-Dimethyl-1H-pyrazol-5-yl)sulfonyl)-2-azaspiro[3.4]octan-6-yl)morpholine (Example 36, 254 mg, 0.7 mmol) was further purified by SFC using a Chiralpak AD-H 30×250 mm, 5 μm column using 40% MeOH with 0.1% DEA in CO₂. Flow rate: 100 mL/min; ABPR 120 bar; MBPR 40 psi, column temperature 40 °C to afford the following compounds that were concentrated to dryness and then lyophilized:

Peak 1, (R)-4-(2-((1,3-dimethyl-1H-pyrazol-5-yl)sulfonyl)-2-azaspiro[3.4]octan-6-yl)morpholine or (S)-4-(2-((1,3-dimethyl-1H-pyrazol-5-yl)sulfonyl)-2-azaspiro[3.4]octan-6-yl)morpholine as a tan oil (95 mg, 34%). LCMS m/z=355.2 [M+H]⁺. ¹H NMR (500 MHz, DMSO-d₆): δ ppm 6.70 (s, 1H), 3.96 (s, 3H), 3.66 (s, 2H), 3.64-3.59 (m, 2H), 3.55-3.50 (m, 4H), 2.46-2.39 (m, 1H), 2.35-2.23 (m, 4H), 2.21 (s, 3H), 1.84 (dd, J=12.8, 7.0 Hz, 1H), 1.75-1.68 (m, 2H), 1.64-1.57 (m, 1H), 1.48 (dd, J=13.0, 8.7 Hz, 1H), 1.39-1.29 (m, 1H).

Peak 2, (S)-4-(2-((1,3-dimethyl-1H-pyrazol-5-yl)sulfonyl)-2-azaspiro[3.4]octan-6-yl)morpholine or (R)-4-(2-((1,3-dimethyl-1H-pyrazol-5-yl)sulfonyl)-2-azaspiro[3.4]octan-6-yl)morpholine as a tan oil (68 mg, 24%). LCMS m/z=355.2 [M+H]⁺. ¹H NMR (500 MHz, DMSO-d₆): δ ppm 6.70 (s, 1H), 3.96 (s, 3H), 3.66 (s, 2H), 3.65-3.59 (m, 2H), 3.52 (br s, 4H), 2.47-2.39 (m, 1H), 2.39-2.23 (m, 4H), 2.21 (s, 3H), 1.84 (br dd, J=12.8, 7.0 Hz, 1H), 1.75-1.68 (m, 2H), 1.65-1.56 (m, 1H), 1.52-1.45 (m, 1H), 1.40-1.30 (m, 1H).

Stereochemistry of the isomers was arbitrarily assigned.

### Example 68

### 6-(2-((6-(1,1-difluoroethyl)-2-methylpyridin-3-yl)sulfonyl)-2-azaspiro[3.3]heptan-6-yl)-1-oxa-6-azaspiro [3.3]heptane

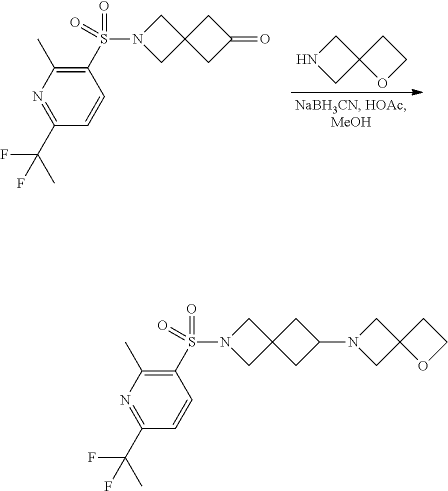 [SMILES: Cc1nc(C(C)(F)F)ccc1S(=O)(=O)N1CC2(CC(=O)C2)C1 ; C1CC2(CNC2)O1 ; Cc1nc(C(C)(F)F)ccc1S(=O)(=O)N1CC2(CC(N3CC4(CCO4)C3)C2)C1]

6-(2-((6-(1,1-difluoroethyl)-2-methylpyridin-3-yl)sulfonyl)-2-azaspiro[3.3]heptan-6-yl)-1-oxa-6-azaspiro[3.3]heptane was obtained (67 mg, 54%), from 2-((6-(1,1-difluoroethyl)-2-methylpyridin-3-yl)sulfonyl)-2-azaspiro[3.3]heptan-6-one (100 mg, 302.71 μmol) and 1-oxa-6-azaspiro[3.3]heptane (73.65 mg, 393.52 μmol, Oxalate), following the procedure described in Example 1, step 6. LCMS m/z=414.1 [M+H]⁺. ¹HNMR (400 MHz, CDCl₃): δ ppm 8.27 (d, J=8.0 Hz, 1H), 7.59 (d, J=8.0 Hz, 1H), 4.50 (t, J=8.0 Hz 2H), 3.89 (s, 2H), 3.84 (s, 2H), 3.50-3.47 (m, 2H), 3.07-3.05 (m, 2H), 2.95-2.88 (m, 1H), 2.85 (s, 3H), 2.82 (t, J=7.6 Hz, 2H), 2.23-2.18 (m, 2H), 2.01 (t, J=18.8 Hz, 3H), 1.95-1.90 (m, 2H).

### Example 69

### 2-((6-(1,1-difluoroethyl)-2-methylpyridin-3-yl)sulfonyl)-N-(oxetan-3-yl)-2-azaspiro[3.3]heptan-6-amine

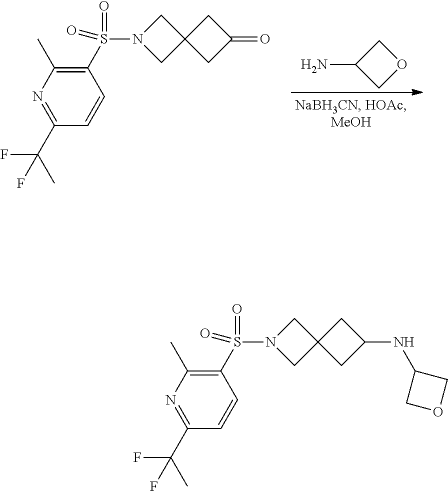 [SMILES: Cc1nc(C(C)(F)F)ccc1S(=O)(=O)N1CC2(CC(=O)C2)C1 ; NC1COC1 ; Cc1nc(C(C)(F)F)ccc1S(=O)(=O)N1CC2(CC(NC3COC3)C2)C1]

2-((6-(1,1-difluoroethyl)-2-methylpyridin-3-yl)sulfonyl)-N-(oxetan-3-yl)-2-azaspiro[3.3]heptan-6-amine was obtained (150 mg, 64%), was obtained from 2-((6-(1,1-difluoroethyl)-2-methylpyridin-3-yl)sulfonyl)-2-azaspiro[3.3]heptan-6-one (200 mg, 605.4 μmol) and oxetan-3-amine (66.4 mg, 908.1 μmol) following the procedure described in Example 1, step 6. LCMS m/z=388.1 [M+H]⁺. ¹HNMR (400 MHz, CDCl3): δ ppm 8.27 (d, J=8.0 Hz, 1H), 7.59 (d, J=8.0 Hz, 1H), 4.78 (t, J=6.8 Hz, 2H), 4.39 (t, J=6.4 Hz, 2H), 3.93-3.88 (m, 3H), 3.85 (s, 2H), 3.19-3.12 (m, 1H), 2.86 (s, 3H), 2.44-2.39 (m, 2H), 2.01 (t, J=18.8 Hz, 3H), 1.91-1.86 (m, 2H).

### Example 70

### 2-((6-(1,1-difluoroethyl)-2-methylpyridin-3-yl)sulfonyl)-N-(2-oxaspiro[3.3]heptan-6-yl)-2-azaspiro[3.3]heptan-6-amine

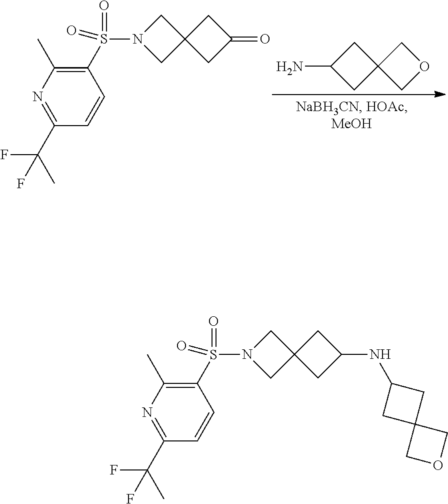

2-((6-(1,1-difluoroethyl)-2-methylpyridin-3-yl)sulfonyl)-N-(2-oxaspiro[3.3]heptan-6-yl)-2-azaspiro[3.3]heptan-6-amine (160 mg, 62%), was obtained from 2-((6-(1,1-difluoroethyl)-2-methylpyridin-3-yl)sulfonyl)-2-azaspiro[3.3]heptan-6-one (200 mg, 605.4 μmol) and 2-oxaspiro[3.3]heptan-6-amine (108.70 mg, 726.50 μmol, HCl salt) following the procedure described in Example 1, step 6. LCMS m/z=428.1 [M+H]⁺. ¹HNMR (400 MHz, CDCl3): δ ppm 8.27 (d, J=8.0 Hz, 1H), 7.59 (d, J=8.4 Hz, 1H), 4.68 (s, 2H), 4.57 (s, 2H), 3.92 (s, 2H), 3.83 (s, 2H), 3.14-2.97 (m, 2H), 2.86 (s, 3H), 2.51-2.39 (m, 4H), 2.01 (t, J=18.8 Hz, 3H), 1.88-1.81 (m, 4H).

### Example 71

### 2-((6-(1,1-difluoroethyl)-2-methylpyridin-3-yl)sulfonyl)-N-((tetrahydro-2H-pyran-4-yl)methyl)-2-azaspiro[3.3]heptan-6-amine

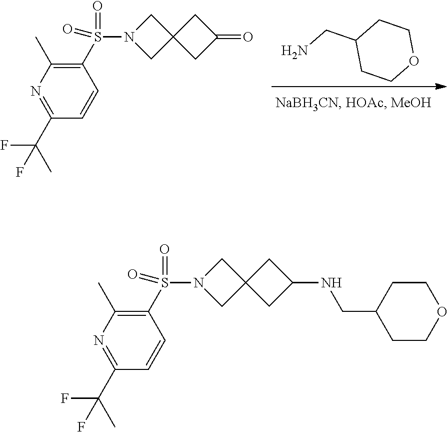 [SMILES: Cc1nc(C(C)(F)F)ccc1S(=O)(=O)N1CC2(CC(=O)C2)C1 ; NCC1CCOCC1 ; Cc1nc(C(C)(F)F)ccc1S(=O)(=O)N1CC2(CC(NCC3CCOCC3)C2)C1]

2-((6-(1,1-difluoroethyl)-2-methylpyridin-3-yl)sulfonyl)-N-((tetrahydro-2H-pyran-4-yl)methyl)-2-azaspiro[3.3]heptan-6-amine (43 mg, 33%) was obtained from 2-((6-(1,1-difluoroethyl)-2-methylpyridin-3-yl)sulfonyl)-2-azaspiro[3.3]heptan-6-one (100 mg, 302.7 μmol) and (tetrahydro-2H-pyran-4-yl)methanamine (38.35 mg, 332.98 μmol) following the procedure described in Example 1, step 6. LCMS m/z=430.1 [M+H]⁺. ¹HNMR (400 MHz, CDCl3): δ ppm 8.28 (d, J=8.4 Hz, 1H), 7.59 (d, J=8.4 Hz, 1H), 3.97-3.93 (m, 4H), 3.86 (s, 2H), 3.39-3.33 (m, 2H), 3.18-3.11 (m, 1H), 2.86 (s, 3H), 2.47-2.42 (m, 2H), 2.36 (d, J=6.0 Hz, 2H), 2.01 (t, J=18.8 Hz, 3H), 1.89-1.84 (m, 2H), 1.66-1.62 (m, 2H), 1.55-1.49 (m, 1H), 1.31-1.21 (m, 2H).

### Example 72

### 2-((6-(1,1-difluoroethyl)-2-methylpyridin-3-yl)sulfonyl)-N-methyl-N-(tetrahydro-2H-pyran-4-yl)-2-azaspiro[3.3]heptan-6-amine

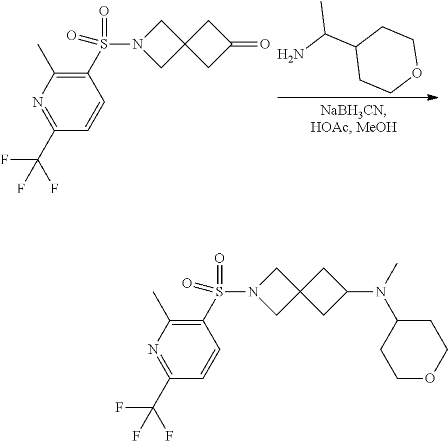

2-((6-(1,1-difluoroethyl)-2-methylpyridin-3-yl)sulfonyl)-N-methyl-N-(tetrahydro-2H-pyran-4-yl)-2-azaspiro[3.3]heptan-6-amine (68 mg, 52%) was obtained from 2-((6-(1,1-difluoroethyl)-2-methylpyridin-3-yl)sulfonyl)-2-azaspiro[3.3]heptan-6-one (100 mg, 302.7 μmol) and 1-(tetrahydro-2H-pyran-4-yl)ethan-1-amine (104.59 mg, 908.13 μmol) following the procedure described in Example 1, step 6. LCMS m/z=430.2 [M+H]⁺. ¹HNMR (400 MHz, CDCl3): δ ppm 8.28 (d, J=8.0 Hz, 1H), 7.60 (d, J=8.0 Hz, 1H), 4.03-3.99 (m, 2H), 3.95 (s, 2H), 3.83 (s, 2H), 3.37-3.31 (m, 2H), 3.04-3.00 (m, 1H), 2.86 (s, 3H), 2.62-2.57 (m, 1H), 2.34-2.29 (m, 2H), 2.10-2.07 (m, 3H), 2.01 (t, J=18.8 Hz, 3H), 1.66-1.60 (m, 3H), 1.58-1.55 (m, 3H).

### Example 73

### 2-((6-(1,1-difluoroethyl)-2-methylpyridin-3-yl)sulfonyl)-N-methyl-N-(oxetan-3-yl)-2-azaspiro[3.3]heptan-6-amine

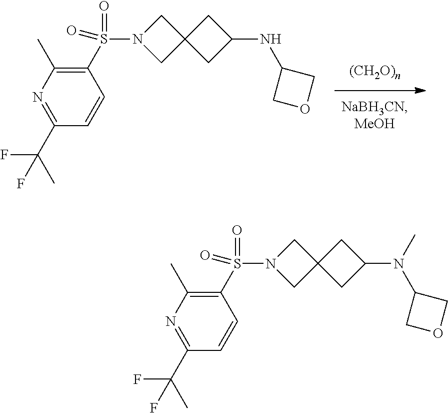 [SMILES: Cc1nc(C(C)(F)F)ccc1S(=O)(=O)N1CC2(CC(NC3COC3)C2)C1 ; Cc1nc(C(C)(F)F)ccc1S(=O)(=O)N1CC2(CC(N(C)C3COC3)C2)C1]

2-((6-(1,1-difluoroethyl)-2-methylpyridin-3-yl)sulfonyl)-N-methyl-N-(oxetan-3-yl)-2-azaspiro[3.3]heptan-6-amine (55 mg, 53%) was obtained from 2-((6-(1,1-difluoroethyl)-2-methylpyridin-3-yl)sulfonyl)-2-azaspiro[3.3]heptan-6-one (100 mg, 302.7 μmol) and (CH₂O)ₙ(70.3 mg, 2.0 mmol) following the procedure described in Example 59. LCMS m/z=402.1 [M+H]⁺. ¹HNMR (400 MHz, CDCl3): δ ppm 8.27 (d, J=8.0 Hz, 1H), 7.59 (d, J=8.4 Hz, 1H), 4.64 (t, J=6.4 Hz, 2H), 4.56 (t, J=6.4 Hz, 2H), 3.93 (s, 2H), 3.82 (s, 2H), 3.63-3.56 (m, 1H), 2.86 (s, 3H), 2.69-2.59 (m, 1H), 2.26-2.21 (m, 2H), 2.07-1.96 (m, 8H).

### Example 74

### 2-((6-(1,1-difluoroethyl)-2-methylpyridin-3-yl)sulfonyl)-N-methyl-N-(2-oxaspiro[3.3]heptan-6-yl)-2-azaspiro[3.3]heptan-6-amine

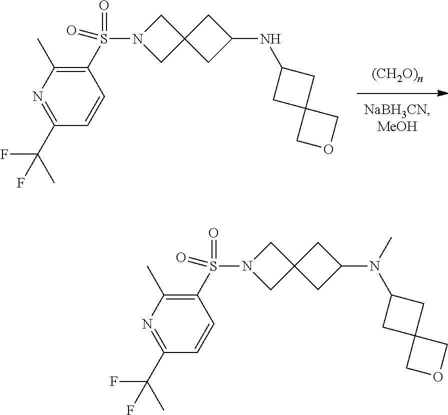 [SMILES: Cc1nc(C(C)(F)F)ccc1S(=O)(=O)N1CC2(CC(NC3CC4(COC4)C3)C2)C1 ; Cc1nc(C(C)(F)F)ccc1S(=O)(=O)N1CC2(CC(N(C)C3CC4(COC4)C3)C2)C1]

2-((6-(1,1-difluoroethyl)-2-methylpyridin-3-yl)sulfonyl)-N-methyl-N-(2-oxaspiro[3.3]heptan-6-yl)-2-azaspiro[3.3]heptan-6-amine (158 mg, 67%) was obtained from 2-((6-(1,1-difluoroethyl)-2-methylpyridin-3-yl)sulfonyl)-N-(2-oxaspiro[3.3]heptan-6-yl)-2-azaspiro[3.3]heptan-6-amine (100 mg, 233.9 μmol) and (CH₂O)ₙ(79.7 mg, 2.34 mmol) following the procedure described in Example 59. LCMS m/z=442.2 [M+H]⁺. ¹HNMR (400 MHz, CDCl3): δ ppm 8.27 (d, J=8.0 Hz, 1H), 7.59 (d, J=8.0 Hz, 1H), 4.68 (s, 2H), 4.55 (s, 2H), 3.93 (s, 2H), 3.80 (s, 2H), 2.86 (s, 3H), 2.63-2.46 (m, 2H), 2.34-2.23 (m, 4H), 2.08-1.96 (m, 7H), 1.92 (s, 3H).

### Example 75

### 2-((6-(1,1-difluoroethyl)-2-methylpyridin-3-yl)sulfonyl)-N-methyl-N-((tetrahydro-2H-pyran-4-yl)methyl)-2-azaspiro[3.3]heptan-6-amine

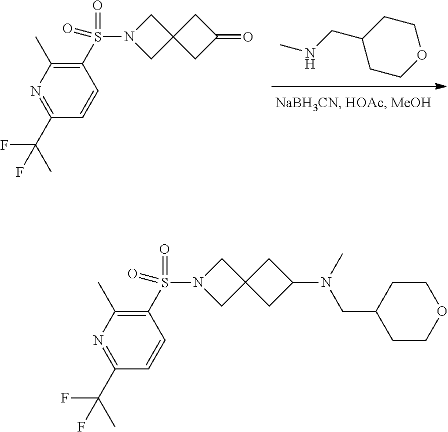

2-((6-(1,1-difluoroethyl)-2-methylpyridin-3-yl)sulfonyl)-N-methyl-N-((tetrahydro-2H-pyran-4-yl)methyl)-2-azaspiro[3.3]heptan-6-amine (71 mg, 53%) was obtained from 2-((6-(1,1-difluoroethyl)-2-methylpyridin-3-yl)sulfonyl)-2-azaspiro[3.3]heptan-6-one (100 mg, 302.7 μmol) and N-methyl-1-(tetrahydro-2H-pyran-4-yl)methanamine (50.8 mg, 394 μmol) following the procedure described in Example 1, step 6. LCMS m/z=444.2 [M+H]⁺. ¹HNMR (400 MHz, CDCl3): δ ppm 8.28 (d, J=8.0 Hz, 1H), 7.59 (d, J=8.4 Hz, 1H), 3.96-3.92 (m, 4H), 3.83 (s, 2H), 3.39-3.33 (m, 2H), 2.86 (s, 3H), 2.60-2.54 (m, 1H), 2.30-2.25 (m, 2H), 2.06-1.97 (m, 10H), 1.64-1.62 (m, 3H), 1.24-1.14 (m, 2H).

### Example 76

### 2-((4-methyl-2-(trifluoromethyl)pyrimidin-5-yl)sulfonyl)-N-(tetrahydro-2H-pyran-4-yl)-2-azaspiro[3.3]heptan-6-amine

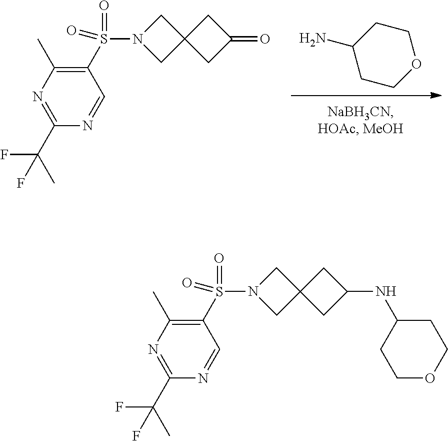 [SMILES: Cc1nc(C(C)(F)F)ncc1S(=O)(=O)N1CC2(CC(=O)C2)C1 ; NC1CCOCC1 ; Cc1nc(C(C)(F)F)ncc1S(=O)(=O)N1CC2(CC(NC3CCOCC3)C2)C1]

2-((4-methyl-2-(trifluoromethyl)pyrimidin-5-yl)sulfonyl)-N-(tetrahydro-2H-pyran-4-yl)-2-azaspiro[3.3]heptan-6-amine (15 mg, 17%) was obtained from 2-((4-methyl-2-(trifluoromethyl)pyrimidin-5-yl)sulfonyl)-2-azaspiro[3.3]heptan-6-one (70 mg, 208.77 μmol) and tetrahydro-2H-pyran-4-amine (63 mg, 626 μmol) following the procedure described in Example 16, step 4. LCMS m/z=421.1 [M+H]⁺. ¹HNMR (400 MHz, CDCl3): δ ppm 9.18 (s, 1H), 4.03 (s, 2H), 3.97-3.93 (m, 4H), 3.40-3.29 (m, 3H), 2.91 (s, 3H), 2.66-2.62 (m, 1H), 2.53-2.48 (m, 2H), 1.95-1.90 (m, 2H), 1.74-1.71 (m, 2H), 1.41-1.31 (m, 2H).

### Example 77

### 2-((4-methyl-2-(trifluoromethyl)pyrimidin-5-yl)sulfonyl)-N-(oxetan-3-yl)-2-azaspiro[3.3]heptan-6-amine

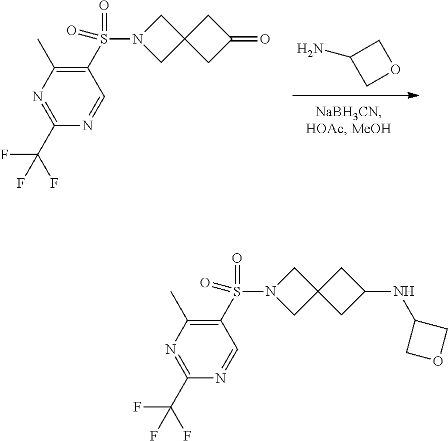 [SMILES: Cc1nc(C(F)(F)F)ncc1S(=O)(=O)N1CC2(CC(=O)C2)C1 ; NC1COC1 ; Cc1nc(C(F)(F)F)ncc1S(=O)(=O)N1CC2(CC(NC3COC3)C2)C1]

2-((4-methyl-2-(trifluoromethyl)pyrimidin-5-yl)sulfonyl)-N-(oxetan-3-yl)-2-azaspiro[3.3]heptan-6-amine (115 mg, 54%) was obtained from 2-((4-methyl-2-(trifluoromethyl)pyrimidin-5-yl)sulfonyl)-2-azaspiro[3.3]heptan-6-one (180 mg, 536.8 μmol) and oxetan-3-amine (47 mg, 644 μmol) following the procedure described in Example 16, step 4. LCMS m/z=393.1 [M+H]⁺. ¹HNMR (400 MHz, CDCl3): δ ppm 9.17 (s, 1H), 4.78 (t, J=7.0 Hz, 2H), 4.39 (t, J=6.5 Hz, 2H), 4.01 (s, 2H), 3.96 (s, 2H), 3.94-3.86 (m, 1H), 3.22-3.14 (m, 1H), 2.90 (s, 3H), 2.47-2.40 (m, 2H), 1.95-1.88 (m, 2H).

### Example 78

### 2-((4-methyl-2-(trifluoromethyl)pyrimidin-5-yl)sulfonyl)-N-(2-oxaspiro[3.3]heptan-6-yl)-2-azaspiro[3.3]heptan-6-amine

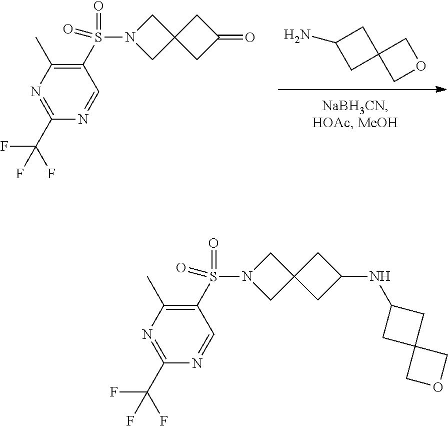 [SMILES: Cc1nc(C(F)(F)F)ncc1S(=O)(=O)N1CC2(CC(=O)C2)C1 ; NC1CC2(COC2)C1 ; Cc1nc(C(F)(F)F)ncc1S(=O)(=O)N1CC2(CC(NC3CC4(COC4)C3)C2)C1]

2-((4-methyl-2-(trifluoromethyl)pyrimidin-5-yl)sulfonyl)-N-(2-oxaspiro[3.3]heptan-6-yl)-2-azaspiro[3.3]heptan-6-amine (100 mg, 47%) was obtained from 2-((4-methyl-2-(trifluoromethyl)pyrimidin-5-yl)sulfonyl)-2-azaspiro[3.3]heptan-6-one (160 mg, 477.1 μmol) and 2-oxaspiro[3.3]heptan-6-amine (92.8 mg, 620 μmol) following the procedure described in Example 16, step 4. LCMS m/z=433.1 [M+H]⁺. ¹HNMR (400 MHz, CDCl3): δ ppm 9.17 (s, 1H), 4.68 (s, 2H), 4.58 (s, 2H), 4.01 (s, 2H), 3.94 (s, 2H), 3.17-3.10 (m, 1H), 3.07-2.98 (m, 1H), 2.90 (s, 3H), 2.53-2.42 (m, 4H), 1.94-1.87 (m, 4H).

### Example 79

### 2-((4-methyl-2-(trifluoromethyl)pyrimidin-5-yl)sulfonyl)-N-((tetrahydro-2H-pyran-4-yl)methyl)-2-azaspiro[3.3]heptan-6-amine

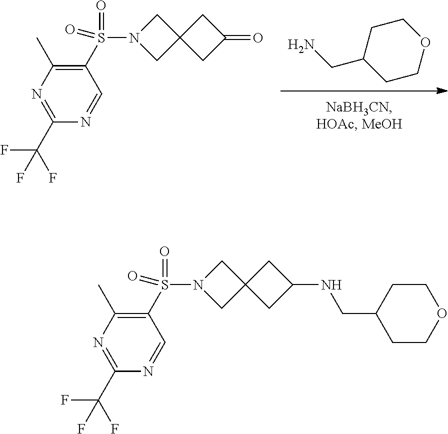 [SMILES: Cc1nc(C(F)(F)F)ncc1S(=O)(=O)N1CC2(CC(=O)C2)C1 ; NCC1CCOCC1 ; Cc1nc(C(F)(F)F)ncc1S(=O)(=O)N1CC2(CC(NCC3CCOCC3)C2)C1]

2-((4-methyl-2-(trifluoromethyl)pyrimidin-5-yl)sulfonyl)-N-((tetrahydro-2H-pyran-4-yl)methyl)-2-azaspiro[3.3]heptan-6-amine (13 mg, 17%) was obtained from 2-((4-methyl-2-(trifluoromethyl)pyrimidin-5-yl)sulfonyl)-2-azaspiro[3.3]heptan-6-one (60 mg, 178.9 μmol) and (tetrahydro-2H-pyran-4-yl)methanamine (24.7 mg, 214.7 μmol) following the procedure described in Example 16, step 4. LCMS m/z=435.1 [M+H]⁺. ¹HNMR (400 MHz, CDCl3): δ ppm 9.18 (s, 1H), 4.03 (s, 2H), 3.98 (s, 2H), 3.96-3.93 (m, 2H), 3.39-3.33 (m, 2H), 3.26-3.22 (m, 1H), 2.90 (s, 3H), 2.53-2.48 (m, 2H), 2.43 (d, J=6.4 Hz, 2H), 2.07-2.06 (m, 1H), 1.75-1.69 (m, 2H), 1.34-1.24 (m, 4H).

### Example 80

### N-methyl-2-((4-methyl-2-(trifluoromethyl)pyrimidin-5-yl)sulfonyl)-N-(tetrahydro-2H-pyran-4-yl)-2-azaspiro[3.3]heptan-6-amine

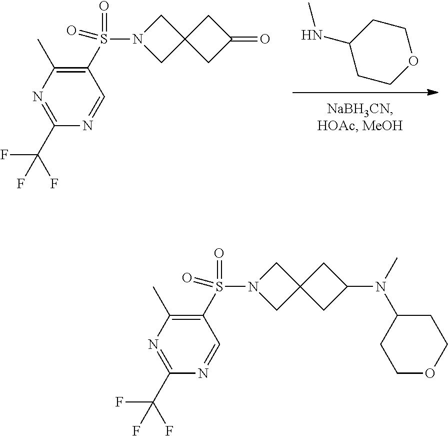

N-methyl-2-((4-methyl-2-(trifluoromethyl)pyrimidin-5-yl)sulfonyl)-N-(tetrahydro-2H-pyran-4-yl)-2-azaspiro[3.3]heptan-6-amine (22 mg, 23%) was obtained from 2-((4-methyl-2-(trifluoromethyl)pyrimidin-5-yl)sulfonyl)-2-azaspiro[3.3]heptan-6-one (70 mg, 208.8 μmol) and N-methyltetrahydro-2H-pyran-4-amine (36.1 mg, 313.1 μmol) following the procedure described in Example 16, step 4. LCMS m/z=435.1 [M+H]⁺. ¹HNMR (400 MHz, CDCl3): δ ppm 9.18 (s, 1H), 4.04-3.99 (m, 4H), 3.93 (s, 2H), 3.37-3.31 (m, 2H), 3.07-2.99 (m, 1H), 2.91 (s, 3H), 2.63-2.57 (m, 1H), 2.36-2.31 (m, 2H), 2.08 (s, 3H), 1.66-1.51 (m, 6H).

### Example 81

### N-methyl-2-((4-methyl-2-(trifluoromethyl)pyrimidin-5-yl)sulfonyl)-N-(oxetan-3-yl)-2-azaspiro[3.3]heptan-6-amine

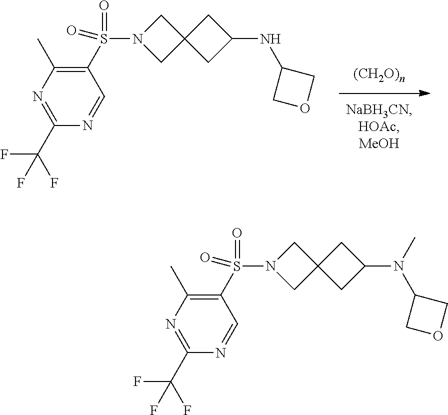 [SMILES: Cc1nc(C(F)(F)F)ncc1S(=O)(=O)N1CC2(CC(NC3COC3)C2)C1 ; Cc1nc(C(F)(F)F)ncc1S(=O)(=O)N1CC2(CC(N(C)C3COC3)C2)C1]

N-methyl-2-((4-methyl-2-(trifluoromethyl)pyrimidin-5-yl)sulfonyl)-N-(oxetan-3-yl)-2-azaspiro[3.3]heptan-6-amine (35 mg, 67.6%) was obtained from 2-((4-methyl-2-(trifluoromethyl)pyrimidin-5-yl)sulfonyl)-N-(oxetan-3-yl)-2-azaspiro[3.3]heptan-6-amine (50 mg, 127.4 μmol) and (CH₂O)ₙ(764 mg, 637 μmol) following the procedure described in Example 59. LCMS m/z=407.1 [M+H]⁺. ¹HNMR (400 MHz, CDCl3): δ ppm 9.17 (s, 1H), 4.64 (t, J=6.5 Hz, 2H), 4.56 (t, J=6.5 Hz, 2H), 4.02 (s, 2H), 3.92 (s, 2H), 3.63-3.57 (m, 1H), 2.90 (s, 3H), 2.68-2.62 (m, 1H), 2.28-2.22 (m, 2H), 2.10-2.05 (m, 2H), 2.04 (s, 3H).

### Example 82

### N-methyl-2-((4-methyl-2-(trifluoromethyl)pyrimidin-5-yl)sulfonyl)-N-(2-oxaspiro[3.3]heptan-6-yl)-2-azaspiro[3.3]heptan-6-amine

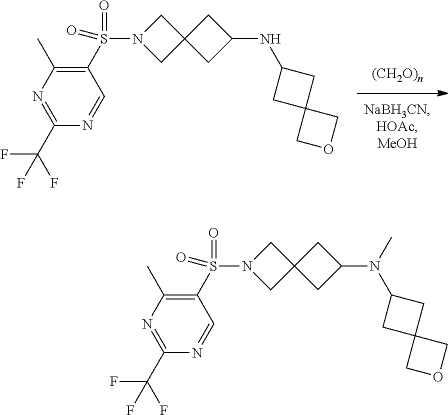 [SMILES: Cc1nc(C(F)(F)F)ncc1S(=O)(=O)N1CC2(CC(NC3CC4(COC4)C3)C2)C1 ; Cc1nc(C(F)(F)F)ncc1S(=O)(=O)N1CC2(CC(N(C)C3CC4(COC4)C3)C2)C1]

N-methyl-2-((4-methyl-2-(trifluoromethyl)pyrimidin-5-yl)sulfonyl)-N-(2-oxaspiro[3.3]heptan-6-yl)-2-azaspiro[3.3]heptan-6-amine (20 mg. 24%) was obtained from 2-((4-methyl-2-(trifluoromethyl)pyrimidin-5-yl)sulfonyl)-N-(2-oxaspiro[3.3]heptan-6-yl)-2-azaspiro[3.3]heptan-6-amine (80 mg, 185 μmol) and (CH₂O)ₙ(63 mg, 1.85 mmol) following the procedure described in Example 59. LCMS m/z=447.1 [M+H]⁺. ¹HNMR (400 MHz, CDCl3): δ ppm 9.17 (s, 1H), 4.69 (s, 2H), 4.56 (s, 2H), 4.03 (s, 2H), 3.92 (s, 2H), 2.90 (s, 3H), 2.63-2.49 (m, 2H), 2.33-2.29 (m, 4H), 2.11-2.01 (m, 4H), 1.94 (s, 3H).

### Example 83

### N-methyl-2-((4-methyl-2-(trifluoromethyl)pyrimidin-5-yl)sulfonyl)-N-((tetrahydro-2H-pyran-4-yl)methyl)-2-azaspiro[3.3]heptan-6-amine

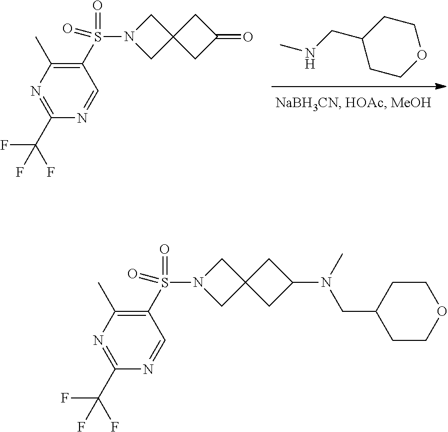

N-methyl-2-((4-methyl-2-(trifluoromethyl)pyrimidin-5-yl)sulfonyl)-N-((tetrahydro-2H-pyran-4-yl)methyl)-2-azaspiro[3.3]heptan-6-amine (38 mg, 45.6%) was obtained from 2-((4-methyl-2-(trifluoromethyl)pyrimidin-5-yl)sulfonyl)-2-azaspiro[3.3]heptan-6-one (60 mg, 178.9 μmol) and N-methyl-1-(tetrahydro-2H-pyran-4-yl)methanamine (27.7 mg, 214.7 μmol) following the procedure described in Example 16, step 4. LCMS m/z=449.2 [M+H]⁺. ¹HNMR (400 MHz, CDCl3): δ ppm 9.18 (s, 1H), 4.01 (s, 2H), 3.99-3.93 (m, 4H), 3.40-3.34 (m, 2H), 2.91 (s, 3H), 2.64-2.56 (m, 1H), 2.33-2.28 (m, 2H), 2.06 (s, 3H), 2.02-1.95 (m, 4H), 1.69-1.67 (m, 1H), 1.67-1.64 (m, 2H), 1.24-1.14 (m, 2H).

### Assays

### EBP Functional Assay

The EBP immunoaffinity (IA) LC-MS assay measures the potency of small molecule inhibitors of EBP by quantifying their concentration-dependent changes in the enzyme's substrate and product using liquid chromatography atmospheric pressure chemical ionization multiple reaction monitoring mass spectrometry (LC-APCI MRM MS). HEK293T cells were utilized as the source of EBP enzyme. The enzyme was incubated with the small molecule inhibitors at variable concentrations for 30 min. Deuterated form of EBP substrate, zymosterol-d5 (Avanti Polar Lipids, Cat #700068P-1 mg), was then added and the plate was incubated at 37 °C for 4 h. Finally, the sterol isomers were extracted and injected to LC-APCI MRM MS. MRM transition used for the quantification for both zymosterol and dihydrolathosterol (substrate and product of EBP enzymatic reaction, respectively) is 372.3-203.2, CE 30 and DP 80 in positive ion mode. Percent conversion of the zymosterol-d5 to dehydrolathosterol-d5 was used to derive IC₅₀ curves. Tasin-1 (1′-[(4-Methoxyphenyl)sulfonyl]-4-methyl-1,4′-bipiperidine, CAS 792927-06-1) was used as the reference small molecule inhibitor.

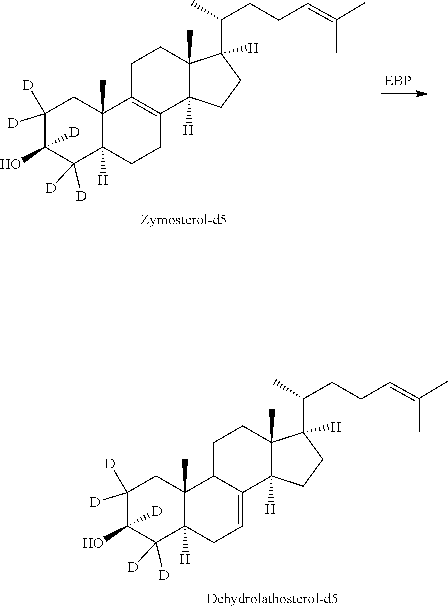

Percent conversion versus the compound concentration data were fit to the following 4-parameter logistic model to generate IC₅₀ curves:

$$y = LA + \frac{UA - LA}{1 + 1 {0}^{( log {IC}_{50} - x ) \cdot Slope}}$$

### Data for Examples

| Example Number | EBP Functional Assay* |
|---|---|
| 1 | +++ |
| 2 | +++ |
| 3 | ++ |
| 4 | + |
| 5 | ++ |
| 6 | + |
| 7 | ++ |
| 8 | + |
| 9 | +++ |
| 10 | ++ |
| 11 | ++ |
| 12 | +++ |
| 13 | + |
| 14 | + |
| 15 | + |
| 16 | + |
| 17 | N/A |
| 18 | + |
| 19 | +++ |
| 20 | + |
| 21 | + |
| 22 | + |
| 23 | + |
| 24 | ++ |
| 25 | + |
| 26 | + |
| 27 | + |
| 28 | + |
| 29 | + |
| 30 | + |
| 31 | ++ |
| 32 | +++ |
| 33 | + |
| 34 | ++ |
| 35 | ++ |
| 36 | ++ |
| 37 | ++ |
| 38 | ++ |
| 39 | +++ |
| 40 | +++ |
| 41 | ++ |
| 42 | +++ |
| 43 | +++ |
| 44 | + |
| 45 | +++ |
| 46 | + |
| 47 | ++ |
| 48 | +++ |
| 49 | ++ |
| 50 | + |
| 51 | ++ |
| 52 | + |
| 53 | ++ |
| 54 | ++ |
| 55 | ++ |
| 56 | +++ |
| 57 | + |
| 58 | NT |
| 59 | +++ |
| 60 | +++ |
| 61 | ++ |
| 62 | +++ |
| 63 | ++ |
| 64 | +++ |
| 65 | ++ |
| 66 | ++ |
| 67 | + |
| 68 | + |
| 69 | + |
| 70 | ++ |
| 71 | +++ |
| 72 | +++ |
| 73 | ++ |
| 74 | +++ |
| 75 | ++ |
| 76 | ++ |
| 77 | + |
| 78 | ++ |
| 79 | ++ |
| 80 | +++ |
| 81 | ++ |
| 82 | +++ |
| 83 | ++ |

*N/A means >3 μM; + means 3 μM to >0.5 μM; ++ means 0.1-0.5 μM; +++ means <0.1 μM; NT = Not Tested

What is claimed is:

1. A compound represented by Formula (I): 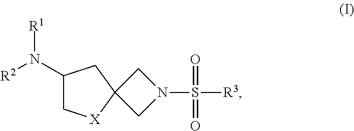
or a pharmaceutically acceptable salt thereof, wherein:
X is O, CH₂, or a bond;
R¹ and R² are each independently selected from H, C₁₋₆alkyl, C₁₋₃haloalkyl, C₃₋₈cycloalkyl, Het, and —Z-Het, wherein the C₁₋₆alkyl, C₃₋₈cycloalkyl, and Het are each optionally substituted with one or more R⁴, provided at least one of R¹ and R² is not H;
or R¹ and R² together with the N atom from which they are attached form a 4 to 7-membered monocyclic heterocycle or 6 to 10-membered bicyclic heterocycle, each of which is optionally substituted with one or more R⁴;
Z is C₁₋₄alkyl optionally substituted with one or more R⁴;
Het is a 4 to 6 membered monocyclic heterocyclyl, or a 6 to 8-membered bicyclic heterocyclyl, each of which is optionally substituted with R⁴;
each R⁴ is independently selected from OR^{4a}, halo, C₁₋₃alkyl, C₁₋₃haloalkyl, and 4 to 7-membered monocyclic heterocycle;
R^{4a} is selected from H, C₁₋₃alkyl, and C₁₋₃haloalkyl;
R³ is phenyl, 5 or 6-membered monocyclic heteroaryl, 9 or 10-membered bicyclic heteroaryl, or 6 to 10 membered bicyclic heterocycle, wherein the phenyl, 5 or 6-membered monocyclic heteroaryl, 9 or 10-membered bicyclic heteroaryl, and 6 to 10 membered bicyclic heterocycle are each optionally substituted with one or more substituent R⁵;
each R⁵ is independently selected from C₁₋₃alkyl, C₁₋₃haloalkyl, OR^{5a}, cyano, and halo;
R^{5a} is selected from H, C₁₋₃alkyl, and C₃₋₆cycloalkyl, wherein the C₁₋₃alkyl is optionally substituted with one or more halo;
provided that R¹ and R² together with the N atom from which they are attached do not form an unsubstituted pyrrolidine, unsubstituted piperidine, or 4-methyl piperazine.

2. The compound of claim 1, or a pharmaceutically acceptable salt thereof, wherein R³ is phenyl or 5 or 6-membered monocyclic heteroaryl, each of which are each optionally substituted with one to three substituent R⁵.

3. The compound of claim 2, or a pharmaceutically acceptable salt thereof, wherein R³ is phenyl, pyridinyl, pyrimidinyl, or pyrazoyl, each of which are each optionally substituted with one to three substituent R⁵.

4. The compound of claim 2, or a pharmaceutically acceptable salt thereof, wherein R³ is represented by the following formula: 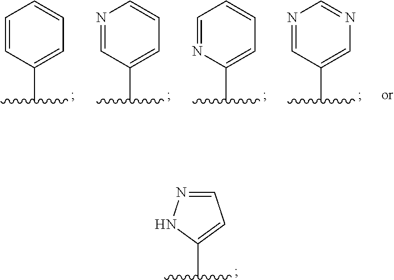
wherein each of the formula depicted above is optionally substituted with one to three R⁵.

5. The compound of any one of claims 1 to 4, or a pharmaceutically acceptable salt thereof, wherein R¹ and R² together with the N atom from which they are attached form a saturated 4 to 6-membered monocyclic heterocycle or a saturated 7 to 10 membered bicyclic heterocycle, each of which is optionally substituted with one or two R⁴.

6. The compound of claim 5, or a pharmaceutically acceptable salt thereof, wherein R¹ and R² together with the nitrogen atom from which they are attached form groups represented by the following formula: 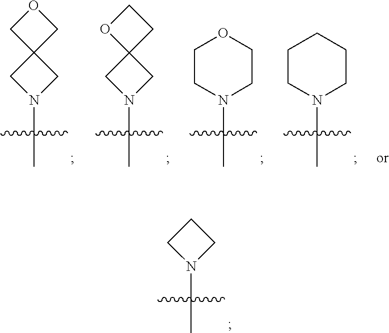
wherein each of the formula depicted above is optionally substituted with one or two R⁴.

7. The compound of any one of claims 1 to 4, or a pharmaceutically acceptable salt thereof, wherein:
R¹ is H or C₁₋₃alkyl optionally substituted with one to three R⁴;
R² is C₁₋₃alkyl, C₃₋₆cycloalkyl, Het, or —Z-Het, wherein the C₁₋₃alkyl, C₃₋₆cycloalkyl; and
Het is a saturated 4 to 6 membered monocyclic heterocycle containing 1 to 2 heteroatoms selected from oxygen and nitrogen, at least one of which is oxygen, wherein the saturated 4 to 6 membered monocyclic heterocycle is optionally substituted with one to three R⁴.

8. The compound of claim 7, or a pharmaceutically acceptable salt thereof, wherein Het is selected from tetraphydropyran and oxetane, each of which is optionally substituted with one to two R⁴.

9. The compound of any one of claims 1 to 8, wherein the compound is represented by Formula (II): 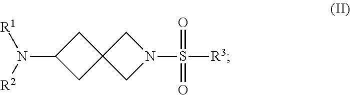
or a pharmaceutically acceptable salt thereof.

10. The compound of claim 9, or a pharmaceutically acceptable salt thereof, wherein R³ is represented by the following formula:

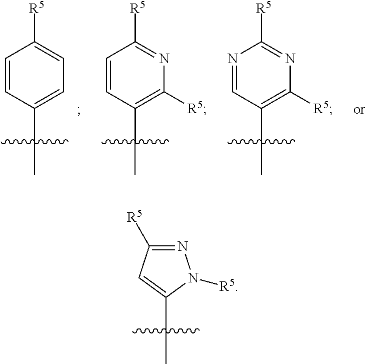

11. The compound of claim 9 or 10, or a pharmaceutically acceptable salt thereof, wherein R⁵, for each occurrence, is independently selected from C₁₋₃alkyl, C₁₋₃haloalkyl, and OR^{5a}.

12. The compound of claim 11, or a pharmaceutically acceptable salt thereof, wherein R⁵, for each occurrence is independently selected from —CH₃, —CF₃, —CF₂CH₃, —CH(CH₃)₂, and —OCHF₂.

13. The compound of any one of claims 9 to 12, or a pharmaceutically acceptable salt thereof, wherein R¹ and R² together with the nitrogen atom from which they are attached form groups represented by the following formula:

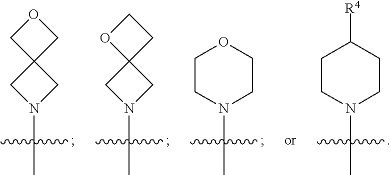

14. The compound of any one of claims 9 to 12, or a pharmaceutically acceptable salt thereof, wherein R¹ is H or —CH₃, and R² is represented by the following formula 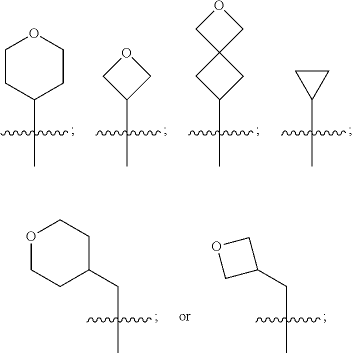
wherein each of the formula depicted above is optionally substituted with one or two R⁴.

15. The compound of any one of claims 9 to 12, or a pharmaceutically acceptable salt thereof, wherein R¹ is H and R² is represented by the following formula 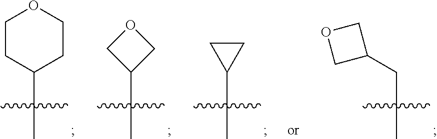
wherein each of the formula depicted above is optionally substituted with one or two R⁴.

16. The compound of claim 14, or a pharmaceutically acceptable salt thereof, wherein R² is represented by the following formula

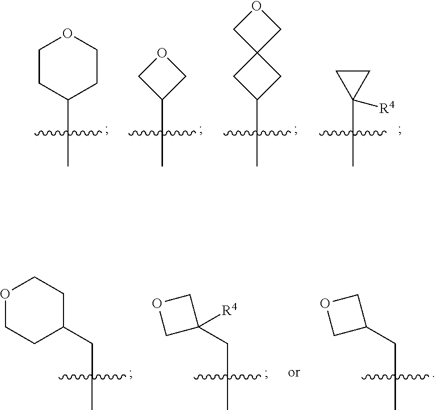

17. The compound of claim 15, or a pharmaceutically acceptable salt thereof, wherein R² is represented by the following formula

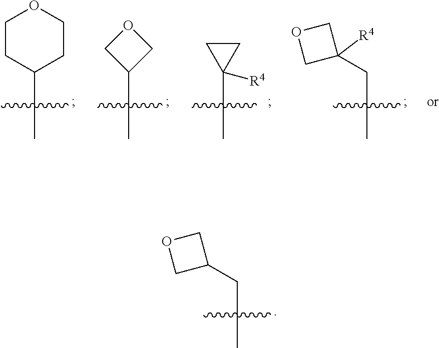

18. The compound of any one of claims 9 to 17, or a pharmaceutically acceptable salt thereof, wherein R⁴, for each occurrence, is independently selected from C₁₋₃alkyl and 4 to 7-membered monocyclic heterocycle.

19. The compound of claim 18, or a pharmaceutically acceptable salt thereof, wherein R⁴, for each occurrence, is independently selected from —CH₃ and tetrahydropyran.

20. The compound of any one of claims 1 to 8, wherein the compound is represented by Formula (III) or (IV): 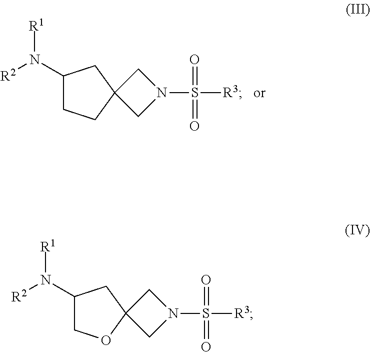
or a pharmaceutically acceptable salt thereof.

21. The compound of claim 20, wherein the compound is represented by Formula (IIIA), (IIIB), (IVA), or (IVA): 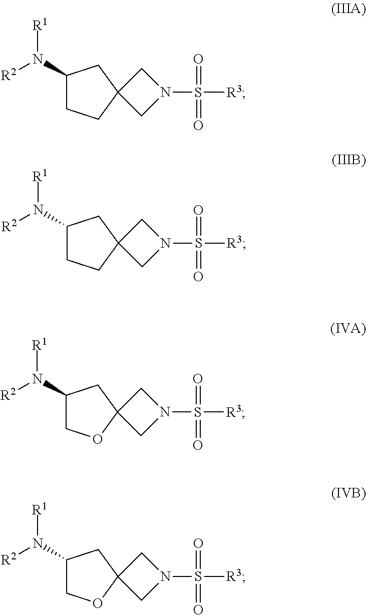
or a pharmaceutically acceptable salt thereof.

22. The compound of claim 20 or 21, or a pharmaceutically acceptable salt thereof, wherein R³ is represented by the following formula:

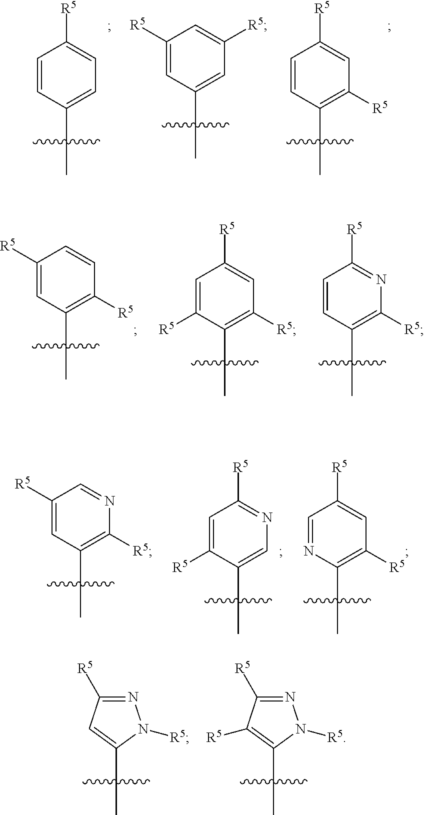

23. The compound of any one of claims 20 to 22, or a pharmaceutically acceptable salt thereof, wherein R⁵, for each occurrence, is independently selected from halo, cyano, C₁₋₂alkyl, C₁₋₂haloalkyl and OR a; and R^{5a} is C₁₋₂alkyl or C₁₋₂haloalkyl.

24. The compound of claim 23, or a pharmaceutically acceptable salt thereof, wherein R⁵, for each occurrence, is independently selected from F, Cl, cyano, —CH₃, —CF₃, —CHF₂, —OCH₃, and —OCHF₂.

25. The compound of any one of claims 20 to 24, or a pharmaceutically acceptable salt thereof, wherein R¹ and R² together with the nitrogen atom from which they are attached form groups represented by the following formula:

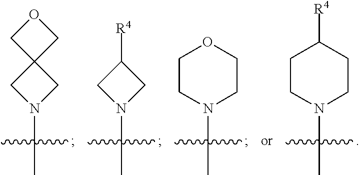

26. The compound of any one of claims 20 to 25, or a pharmaceutically acceptable salt thereof, wherein:
R¹ is H or C₁₋₃alkyl; and
R² is C₁₋₃alkyl, C₃₋₆cycloalkyl, —Z-Het, or Het, each of which is optionally substituted with one to three R⁴.

27. The compound of claim 26, or a pharmaceutically acceptable salt thereof, wherein R¹ is H or —CH₃ and R² is C₁₋₃alkyl optionally substituted with one R⁴, or is represented by the following formula: 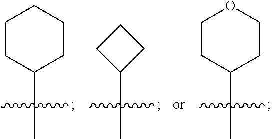
wherein each of the formula depicted above is optionally substituted with one or two R⁴.

28. The compound of claim 27, or a pharmaceutically acceptable salt thereof, wherein R¹ is H or —CH₃ and R² is represented by the following formula:

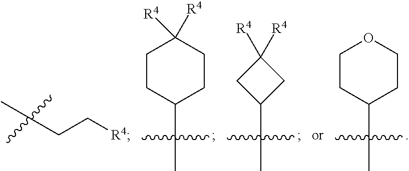

29. The compound of any one of claims 20 to 28, or a pharmaceutically acceptable salt thereof, wherein R⁴, for each occurrence, is independently selected from OR⁴a, halo, and C₁₋₃alkyl, and wherein R^{4a} is C₁₋₃alkyl.

30. The compound of claim 29, or a pharmaceutically acceptable salt thereof, wherein R⁴, for each occurrence, is independently selected from halo, —CH₃, and —OCH₃.

31. The compound of claim 1, or a pharmaceutically acceptable salt thereof, wherein the compound is selected from:
6-(2-((6-(1,1-Difluoroethyl)-2-methylpyridin-3-yl)sulfonyl)-2-azaspiro[3.3]heptan-6-yl)-2-oxa-6-azaspiro[3.3]heptane;
2-((6-(1,1-Difluoroethyl)-2-methylpyridin-3-yl)sulfonyl)-N-(tetrahydro-2H-pyran-4-yl)-2-azaspiro[3.3]heptan-6-amine;
2-((2-Methyl-6-(trifluoromethyl)pyridin-3-yl)sulfonyl)-N-(tetrahydro-2H-pyran-4-yl)-2-azaspiro[3.3]heptan-6-amine;
6-(2-((2-Methyl-6-(trifluoromethyl)pyridin-3-yl)sulfonyl)-2-azaspiro[3.3]heptan-6-yl)-2-oxa-6-azaspiro[3.3]heptane;
4-(2-((2-Methyl-6-(trifluoromethyl)pyridin-3-yl)sulfonyl)-2-azaspiro[3.3]heptan-6-yl)morpholine;
6-(2-((2-Methyl-6-(trifluoromethyl)pyridin-3-yl)sulfonyl)-2-azaspiro[3.3]heptan-6-yl)-1-oxa-6-azaspiro[3.3]heptane;
2-((2-Methyl-6-(trifluoromethyl)pyridin-3-yl)sulfonyl)-N-(1-(tetrahydro-2H-pyran-4-yl)cyclopropyl)-2-azaspiro[3.3]heptan-6-amine;
2-((2-Methyl-6-(trifluoromethyl)pyridin-3-yl)sulfonyl)-N-(oxetan-3-yl)-2-azaspiro[3.3]heptan-6-amine;
2-((2-Methyl-6-(trifluoromethyl)pyridin-3-yl)sulfonyl)-N-((3-methyloxetan-3-yl)methyl)-2-azaspiro[3.3]heptan-6-amine;
4-(2-((1-Methyl-3-(trifluoromethyl)-1H-pyrazol-5-yl)sulfonyl)-2-azaspiro[3.3]heptan-6-yl)morpholine;
2-((1-Methyl-3-(trifluoromethyl)-1H-pyrazol-5-yl)sulfonyl)-N-(oxetan-3-ylmethyl)-2-azaspiro[3.3]heptan-6-amine;
2-((1-Methyl-3-(trifluoromethyl)-1H-pyrazol-5-yl)sulfonyl)-N-((3-methyloxetan-3-yl)methyl)-2-azaspiro[3.3]heptan-6-amine;
2-((1-Methyl-3-(trifluoromethyl)-1H-pyrazol-5-yl)sulfonyl)-N-(oxetan-3-yl)-2-azaspiro[3.3]heptan-6-amine;
6-(2-((1-Methyl-3-(trifluoromethyl)-1H-pyrazol-5-yl)sulfonyl)-2-azaspiro[3.3]heptan-6-yl)-2-oxa-6-azaspiro[3.3]heptane;
6-(2-((1-Methyl-3-(trifluoromethyl)-1H-pyrazol-5-yl)sulfonyl)-2-azaspiro[3.3]heptan-6-yl)-1-oxa-6-azaspiro[3.3]heptane;
6-(2-((4-Methyl-2-(trifluoromethyl)pyrimidin-5-yl)sulfonyl)-2-azaspiro[3.3]heptan-6-yl)-1-oxa-6-azaspiro[3.3]heptane;
6-(2-((4-Methyl-2-(trifluoromethyl)pyrimidin-5-yl)sulfonyl)-2-azaspiro[3.3]heptan-6-yl)-2-oxa-6-azaspiro[3.3]heptane;
2-Methyl-N-(2-(tetrahydro-2H-pyran-4-yl)-2-azaspiro[3.3]heptan-6-yl)-6-(trifluoromethyl)pyridine-3-sulfonamide;
4-(2-((3-Isopropyl-1-methyl-1H-pyrazol-5-yl)sulfonyl)-2-azaspiro[3.3]heptan-6-yl)morpholine;
2-((4-(Difluoromethoxy)phenyl)sulfonyl)-6-(4-methylpiperidin-1-yl)-2-azaspiro[3.3]heptane;
2-((2-Methoxy-5-methylpyridin-3-yl)sulfonyl)-N-(tetrahydro-2H-pyran-4-yl)-5-oxa-2-azaspiro[3.4]octan-7-amine;
2-((2-Methoxy-5-methylpyridin-3-yl)sulfonyl)-7-(2-oxa-6-azaspiro[3.3]heptan-6-yl)-5-oxa-2-azaspiro[3.4]octane;
2-((2-Methoxy-5-methylpyridin-3-yl)sulfonyl)-7-(3-methoxyazetidin-1-yl)-5-oxa-2-azaspiro[3.4]octane;
N-(4,4-Difluorocyclohexyl)-2-((2-methoxy-5-methylpyridin-3-yl)sulfonyl)-5-oxa-2-azaspiro[3.4]octan-7-amine;
N-(3,3-Difluorocyclobutyl)-2-((2-methoxy-5-methylpyridin-3-yl)sulfonyl)-5-oxa-2-azaspiro[3.4]octan-7-amine;
3-((7-((4,4-Difluorocyclohexyl)amino)-5-oxa-2-azaspiro[3.4]octan-2-yl)sulfonyl)-4-fluorobenzonitrile;
4-Fluoro-3-((7-((tetrahydro-2H-pyran-4-yl)amino)-5-oxa-2-azaspiro[3.4]octan-2-yl)sulfonyl)benzonitrile;
3-((7-(2-Oxa-6-azaspiro[3.3]heptan-6-yl)-5-oxa-2-azaspiro[3.4]octan-2-yl)sulfonyl)-5-fluorobenzonitrile;
4-(2-((3,5-Dimethylpyridin-2-yl)sulfonyl)-2-azaspiro[3.4]octan-6-yl)morpholine;
6-(2-((6-Methoxy-2-methylpyridin-3-yl)sulfonyl)-2-azaspiro[3.4]octan-6-yl)-2-oxa-6-azaspiro[3.3]heptane;
N-(4,4-Difluorocyclohexyl)-2-((6-methoxy-2-methylpyridin-3-yl)sulfonyl)-2-azaspiro[3.4]octan-6-amine;
2-((6-Methoxy-2-methylpyridin-3-yl)sulfonyl)-N-(2-methoxyethyl)-N-methyl-2-azaspiro[3.4]octan-6-amine;
N-(2-Methoxyethyl)-N-methyl-2-((1-methyl-3-(trifluoromethyl)-1H-pyrazol-5-yl)sulfonyl)-2-azaspiro[3.4]octan-6-amine;
4-(2-((3-Methoxy-1-methyl-1H-pyrazol-5-yl)sulfonyl)-2-azaspiro[3.4]octan-6-yl)morpholine;
4-(2-((1-Methyl-3-(trifluoromethyl)-1H-pyrazol-5-yl)sulfonyl)-2-azaspiro[3.4]octan-6-yl)morpholine;
4-(2-((1,3-Dimethyl-1H-pyrazol-5-yl)sulfonyl)-2-azaspiro[3.4]octan-6-yl)morpholine;
4-(2-((4-Chloro-1,3-dimethyl-1H-pyrazol-5-yl)sulfonyl)-2-azaspiro[3.4]octan-6-yl)morpholine;
4-(2-((4-(Difluoromethyl)-1,3-dimethyl-1H-pyrazol-5-yl)sulfonyl)-2-azaspiro[3.4]octan-6-yl)morpholine;
4-(2-((6-Methoxy-2-(trifluoromethyl)pyridin-3-yl)sulfonyl)-2-azaspiro[3.4]octan-6-yl)morpholine;
4-(2-((2-Methyl-6-(trifluoromethyl)pyridin-3-yl)sulfonyl)-2-azaspiro[3.4]octan-6-yl)morpholine;
4-(2-((4-Methyl-6-(trifluoromethyl)pyridin-3-yl)sulfonyl)-2-azaspiro[3.4]octan-6-yl)morpholine;
4-(2-((6-Chloro-2-methylpyridin-3-yl)sulfonyl)-2-azaspiro[3.4]octan-6-yl)morpholine;
4-(2-(Mesitylsulfonyl)-2-azaspiro[3.4]octan-6-yl)morpholine;
4-(2-((4-(Difluoromethoxy)phenyl)sulfonyl)-2-azaspiro[3.4]octan-6-yl)morpholine;
4-(2-((2,4-Dimethylphenyl)sulfonyl)-2-azaspiro[3.4]octan-6-yl)morpholine;
4-(2-((2,4-Difluorophenyl)sulfonyl)-2-azaspiro[3.4]octan-6-yl)morpholine;
4-(2-((4,6-Dimethylpyridin-3-yl)sulfonyl)-2-azaspiro[3.4]octan-6-yl)morpholine;
4-(2-((6-methoxy-2-methylpyridin-3-yl)sulfonyl)-2-azaspiro[3.4]octan-6-yl)morpholine;
4-(2-((1-Methyl-3-(trifluoromethyl)-1H-pyrazol-5-yl)sulfonyl)-2-azaspiro[3.4]octan-6-yl)morpholine;
(R)-4-(2-((1-Methyl-3-(trifluoromethyl)-1H-pyrazol-5-yl)sulfonyl)-2-azaspiro[3.4]octan-6-yl)morpholine;
3-Fluoro-5-((6-morpholino-2-azaspiro[3.4]octan-2-yl)sulfonyl)benzonitrile;
(R)-3-Fluoro-5-((6-morpholino-2-azaspiro[3.4]octan-2-yl)sulfonyl)benzonitrile;
4-(2-((1-methyl-3-(trifluoromethyl)-1H-pyrazol-5-yl)sulfonyl)-2-azaspiro[3.4]octan-6-yl)morpholine;
(S)-4-(2-((1-methyl-3-(trifluoromethyl)-1H-pyrazol-5-yl)sulfonyl)-2-azaspiro[3.4]octan-6-yl)morpholine;
3-Fluoro-5-((6-morpholino-2-azaspiro[3.4]octan-2-yl)sulfonyl)benzonitrile;
(S)-3-Fluoro-5-((6-morpholino-2-azaspiro[3.4]octan-2-yl)sulfonyl)benzonitrile;
2-(Mesitylsulfonyl)-7-morpholino-5-oxa-2-azaspiro[3.4]octane;
2-((4-(Difluoromethoxy)phenyl)sulfonyl)-7-(4-methylpiperidin-1-yl)-5-oxa-2-azaspiro[3.4]octane;
2-((1,3-Dimethyl-1H-pyrazol-5-yl)sulfonyl)-7-(4-methylpiperidin-1-yl)-5-oxa-2-azaspiro[3.4]octane;
2-((6-Methoxy-2-methylpyridin-3-yl)sulfonyl)-7-(4-methylpiperidin-1-yl)-5-oxa-2-azaspiro[3.4]octane;
2-((1-methyl-3-(trifluoromethyl)-1H-pyrazol-5-yl)sulfonyl)-7-morpholino-5-oxa-2-azaspiro[3.4]octane;
(R)-2-((1-methyl-3-(trifluoromethyl)-1H-pyrazol-5-yl)sulfonyl)-7-morpholino-5-oxa-2-azaspiro[3.4]octane;
2-((2-Methyl-6-(trifluoromethyl)pyridin-3-yl)sulfonyl)-7-morpholino-5-oxa-2-azaspiro[3.4]octane;
(R)-2-((2-Methyl-6-(trifluoromethyl)pyridin-3-yl)sulfonyl)-7-morpholino-5-oxa-2-azaspiro[3.4]octane;
3-((7-((4,4-Difluorocyclohexyl)(methyl)amino)-5-oxa-2-azaspiro[3.4]octan-2-yl)sulfonyl)-4-fluorobenzonitrile;
4-(2-((2-Methyl-6-(trifluoromethyl)pyridin-3-yl)sulfonyl)-2-azaspiro[3.4]octan-6-yl)morpholine;
(R)-4-(2-((2-Methyl-6-(trifluoromethyl)pyridin-3-yl)sulfonyl)-2-azaspiro[3.4]octan-6-yl)morpholine;
(S)-4-(2-((2-methyl-6-(trifluoromethyl)pyridin-3-yl)sulfonyl)-2-azaspiro[3.4]octan-6-yl)morpholine;
4-(2-((4-Methyl-6-(trifluoromethyl)pyridin-3-yl)sulfonyl)-2-azaspiro[3.4]octan-6-yl)morpholine;
(R)-4-(2-((4-Methyl-6-(trifluoromethyl)pyridin-3-yl)sulfonyl)-2-azaspiro[3.4]octan-6-yl)morpholine;
(S)-4-(2-((4-methyl-6-(trifluoromethyl)pyridin-3-yl)sulfonyl)-2-azaspiro[3.4]octan-6-yl)morpholine;
4-(2-((6-Chloro-2-methylpyridin-3-yl)sulfonyl)-2-azaspiro[3.4]octan-6-yl)morpholine;
(R)-4-(2-((6-Chloro-2-methylpyridin-3-yl)sulfonyl)-2-azaspiro[3.4]octan-6-yl)morpholine;
(S)-4-(2-((6-chloro-2-methylpyridin-3-yl)sulfonyl)-2-azaspiro[3.4]octan-6-yl)morpholine;
4-(2-((1,3-dimethyl-1H-pyrazol-5-yl)sulfonyl)-2-azaspiro[3.4]octan-6-yl)morpholine;
(R)-4-(2-((1,3-dimethyl-1H-pyrazol-5-yl)sulfonyl)-2-azaspiro[3.4]octan-6-yl)morpholine;
(S)-4-(2-((1,3-dimethyl-1H-pyrazol-5-yl)sulfonyl)-2-azaspiro[3.4]octan-6-yl)morpholine;
6-(2-((6-(1,1-difluoroethyl)-2-methylpyridin-3-yl)sulfonyl)-2-azaspiro[3.3]heptan-6-yl)-1-oxa-6-azaspiro[3.3]heptane;
2-((6-(1,1-difluoroethyl)-2-methylpyridin-3-yl)sulfonyl)-N-(oxetan-3-yl)-2-azaspiro[3.3]heptan-6-amine;
2-((6-(1,1-difluoroethyl)-2-methylpyridin-3-yl)sulfonyl)-N-(2-oxaspiro[3.3]heptan-6-yl)-2-azaspiro[3.3]heptan-6-amine;
2-((6-(1,1-difluoroethyl)-2-methylpyridin-3-yl)sulfonyl)-N-((tetrahydro-2H-pyran-4-yl)methyl)-2-azaspiro[3.3]heptan-6-amine;
2-((6-(1,1-difluoroethyl)-2-methylpyridin-3-yl)sulfonyl)-N-methyl-N-(tetrahydro-2H-pyran-4-yl)-2-azaspiro[3.3]heptan-6-amine;
2-((6-(1,1-difluoroethyl)-2-methylpyridin-3-yl)sulfonyl)-N-methyl-N-(oxetan-3-yl)-2-azaspiro[3.3]heptan-6-amine;
2-((6-(1,1-difluoroethyl)-2-methylpyridin-3-yl)sulfonyl)-N-methyl-N-(2-oxaspiro[3.3]heptan-6-yl)-2-azaspiro[3.3]heptan-6-amine;
2-((6-(1,1-difluoroethyl)-2-methylpyridin-3-yl)sulfonyl)-N-methyl-N-((tetrahydro-2H-pyran-4-yl)methyl)-2-azaspiro[3.3]heptan-6-amine;
2-((4-methyl-2-(trifluoromethyl)pyrimidin-5-yl)sulfonyl)-N-(tetrahydro-2H-pyran-4-yl)-2-azaspiro[3.3]heptan-6-amine;
2-((4-methyl-2-(trifluoromethyl)pyrimidin-5-yl)sulfonyl)-N-(oxetan-3-yl)-2-azaspiro[3.3]heptan-6-amine;
2-((4-methyl-2-(trifluoromethyl)pyrimidin-5-yl)sulfonyl)-N-(2-oxaspiro[3.3]heptan-6-yl)-2-azaspiro[3.3]heptan-6-amine;
2-((4-methyl-2-(trifluoromethyl)pyrimidin-5-yl)sulfonyl)-N-((tetrahydro-2H-pyran-4-yl)methyl)-2-azaspiro[3.3]heptan-6-amine;
N-methyl-2-((4-methyl-2-(trifluoromethyl)pyrimidin-5-yl)sulfonyl)-N-(tetrahydro-2H-pyran-4-yl)-2-azaspiro[3.3]heptan-6-amine;
N-methyl-2-((4-methyl-2-(trifluoromethyl)pyrimidin-5-yl)sulfonyl)-N-(oxetan-3-yl)-2-azaspiro[3.3]heptan-6-amine;
N-methyl-2-((4-methyl-2-(trifluoromethyl)pyrimidin-5-yl)sulfonyl)-N-(2-oxaspiro[3.3]heptan-6-yl)-2-azaspiro[3.3]heptan-6-amine; and
N-methyl-2-((4-methyl-2-(trifluoromethyl)pyrimidin-5-yl)sulfonyl)-N-((tetrahydro-2H-pyran-4-yl)methyl)-2-azaspiro[3.3]heptan-6-amine;
or a or a pharmaceutically acceptable salt thereof.

32. A pharmaceutical composition comprising a compound of any one of claims 1 to 31, or a pharmaceutically acceptable salt thereof, and a pharmaceutically acceptable excipient.

33. A method of treating a disease mediated by Emopamil-Binding Protein comprising administering to a subject an effective amount of a compound of any one of claims 1 to 31, or a pharmaceutically acceptable salt thereof, or the pharmaceutical composition of claim 32.

34. A method of treating an autoimmune disease in a subject comprising administering to the subject an effective amount of a compound of any one of claims 1 to 31, or a pharmaceutically acceptable salt thereof, or the pharmaceutical composition of claim 32.

35. The method of claim 34, wherein said autoimmune disease is multiple sclerosis.

36. The method of claim 35, wherein said compound or pharmaceutical composition repairs or forms new myelin sheaths in said subject.
